(19) United States
(12) Patent Application Publication
(10) Pub. No.: US 2024/0182451 A1
(43) Pub. Date: Jun. 6, 2024

(54) AZAINDOLE CYANINE DYES, USES, AND METHODS OF PREPARATION

(71) Applicant: LIFE TECHNOLOGIES CORPORATION, Carlsbad, CA (US)

(72) Inventors: Yi-Zhen HU, Eugene, OR (US); Brian EVANS, Mountain View, CA (US)

(21) Appl. No.: 18/415,904

(22) Filed: Jan. 18, 2024

Related U.S. Application Data
(63) Continuation-in-part of application No. PCT/US2022/073954, filed on Jul. 20, 2022.
(60) Provisional application No. 63/224,302, filed on Jul. 21, 2021.

(51) Int. Cl. C07D 403/06; C07D 471/04; C07D 519/00; C09B 23/04; C09B 23/08; G01N 21/64; G01N 33/533

(52) U.S. Cl. CPC C07D 403/06; C07D 471/04; C07D 519/00; C09B 23/04; C09B 23/083; G01N 21/64; G01N 33/533

(57) ABSTRACT

The present disclosure relates to new and useful azaindole cyanine (pyrrolo pyridine cyanine) compounds. Use of the compounds as dyes that operate in the far-red and near-IR spectral region in biological imaging, and methods of making and using the compounds are also disclosed herein.

### CROSS-REFERENCE TO RELATED APPLICATIONS

This application is a continuation-in-part of International Application No. PCT/US2022/073954, filed Jul. 20, 2022, which claims priority to U.S. Provisional Patent Application No. 63/224,302 filed Jul. 21, 2021, both of which are incorporated herein by reference in their entirety.

### FIELD

Disclosed herein are azaindole cyanine (pyrrolopyridine cyanine) compounds that are useful as dyes, for example, that emit in the far-red and near infrared spectral region. Applications using the cyanine compounds and methods of making same are also described.

### BACKGROUND

Dyes enable researchers to create their own labeled biomolecules for use in a wide variety of imaging applications such immunohistochemistry, fluorescence in situ hybridization (FISH), cell tracing, receptor labeling, and cytochemistry. Dyes may also be used for probing biological structure, function, and interactions.

Unfortunately, dyes that emit in the far red and near-infrared region are far less common. Dyes that operate in the far-red and near-IR spectral regions can be less effective than dyes that emit in the red spectral region for a variety of reasons. For example, certain cyanine dyes that emit into the near and far IR typically can have poor stability. Seven-membered polymethine cyanine dyes are examples of a near IR dye that typically has poor stability due to photobleaching. In addition, dyes that emit in the far IR range typically lack sufficient brightness for use in many types of biological applications. Thus, there is a need in the art for dyes that emit light in the far-read and near-IR spectral regions and that are bright and stable in a variety of thermal and aqueous conditions and over long periods of time.

### BRIEF SUMMARY

The present disclosure relates to new and highly useful azaindole cyanine and pyrrolopyridine cyanine compounds that absorb and/or emit light in the far red and near infrared regions of the electromagnetic spectrum.

Accordingly, the present disclosure relates to compounds chosen from Formula (I) or Formula (II):

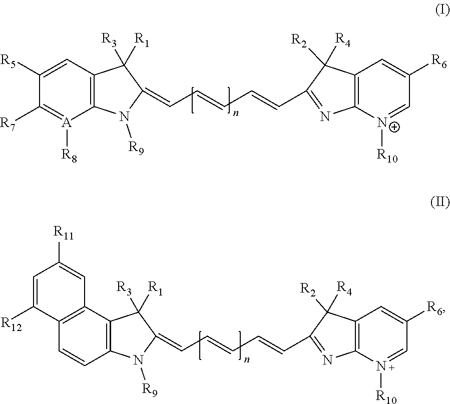

wherein
A is C or N;
n is 0, 1, or 2;
each of R₁, R₂, R₃, and R₄ is the same or different and is independently chosen from alkyl, heteroalkyl, sulfoalkyl group, alkyl with terminal -Su (succinimidyl/sulfosuccinimidyl) ester, heteroalkyl with terminal —SO₃, and alkyl with terminal (Q)_{z}C(O)OR*, and heteroalkyl with terminal (Q)_{z}C(O)OR*,
   where Q is a 5 or 6 membered aryl or heteroaryl ring, z is 0, 1, 2, or 3, R* is chosen from —H, —(CH₂)_{y}SO₃, and N-hydroxysuccinimide, and y is 1, 2, 3, 4, or 5;
each of R₅, R₆, R₁₁, and R₁₂ is the same or different and is independently chosen from —SO₃, -lower alkylene-QR₁₃, -Q(CH₂)_{z}R₁₃, -(Q′)_{y}(R₁₃)_{z}, and -Q″(R₁₃),
   where Q′ is chosen from thiophene, furan, and pyrrole rings, Q″ is a dithienothiophene, and R₁₃ is chosen from —H, —SO₃, —COOH;
R₇ is —H or SO₃;
R₈ is chosen from —H, alkyl, and L^{a}-Z;
or alternatively, R₇ is SRr, and R₈ is alkyl, wherein Rr is an alkylene that forms an unsaturated ring with R₈;
R₉ is chosen from —H, alkyl, sulfoalkyl, L^{a}-Z, and L^{a}-X, on the condition that R₈ and R₉ are not both —H; and
R₁₀ is chosen from alkyl, sulfoalkyl, L^{a}-Z, and L^{a}-X,
   where L^{a} is selected from the group consisting of a divalent linear (—(CH₂)ₒ—, o=0 to 15), crossed, or cyclic alkane group that can be substituted by at least one atom selected from substituted nitrogen and/or sulfur;
   where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur;
   where Z is selected from the group consisting of H, CH₃, alkyl, heteroalkyl, NH₂, —COO—, —COOH, —COSH, CO—NH—NH₂, —COF, —COCl, —COBr, —COI, —COO-Su (succinimidyl/sulfosuccinimidyl) ester, —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO—, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-CHO, —CONR′-L-maleimide, and —CONR′-L-NH—CO—CH₂—I; R′ and R″ is selected from the group consisting of H, alkyl group, and heteroalkyl group, and the biomolecule is a protein, antibody, nucleotide, oligonucleotide, biotin, or hapten;
   X is selected from the group consisting of —SH, —NH₂, —NH—NH₂, —F, —Cl, —Br, I, —NHS (hydroxysuccinimidyl/sulfosuccinimidyl) ester, —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH2-I, where R is —H or an alkyl or heteroalkyl group; and
with the proviso that the compound of Formula (II) cannot be chosen from:

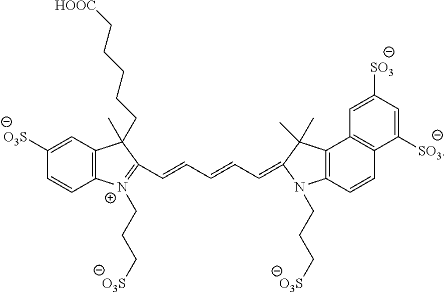

The present disclosure also relates to methods of detecting at least one biomolecule, the method comprising combining at least one biomolecule with a composition comprising at least one excipient and a compound of Formula (I) or (II) in an effective concentration to label at least one biomolecule under conditions sufficient for binding the compound to the biomolecule, and detecting the biomolecule-bound compound.

The present disclosure also relates to a kit for detecting at least one biomolecule in a sample, the kit comprising a compound of Formula (I) or (II) and at least one excipient, and instructions for use of the compound to detect a biomolecule in a sample.

The present disclosure also relates to scientific analysis systems, as well as related computer-implemented methods, computing devices, and computer-readable media, that utilize data generated by processes that involve the novel compounds disclosed herein.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1A-1D show flow cytometry data for labeled and unlabeled cells using Comparator Compound A or Compound B (Compound 41). FIG. 1A=Compound A. FIG. 1B=Compound B.

FIG. 1C=Compound A; cells were fixed and permeabilized. FIG. 1D=Compound B; cells were fixed and permeabilized.

FIG. 2A-2D show flow cytometry data for labeled and unlabeled cells using Compound C (Compound 47) or Compound D (Compound 51). FIG. 2A=Compound C. FIG. 2B=Compound D. FIG. 2C=Compound C; cells were fixed and permeabilized. FIG. 2D=Compound D; cells were fixed and permeabilized.

FIG. 3A-3F show flow cytometry data for Comparator Compound A and Compound B (Compound 41) across three emission channels with excitation at 640 nm. FIG. 3A=Compound A; 663-667 nm. FIG. 3B=Compound A; 705-735 nm. FIG. 3C=Compound A; 750-810 nm. FIG. 3D=Compound B; 663-667 nm. FIG. 3E=Compound B; 705-735 nm. FIG. 3F=Compound B; 750-810 nm.

FIG. 4A-4F show flow cytometry data for Compound C (Compound 47) and Compound D (Compound 51) across three emission channels with excitation at 640 nm. FIG. 4A=Compound C; 663-667 nm. FIG. 4B=Compound C=705-735 nm. FIG. 4C=Compound C; 750-810 nm. FIG. 4D=Compound D; 663-667 nm. FIG. 4E=Compound D; 705-735 nm. FIG. 4F=Compound D; 750-810 nm.

FIG. 5A-5D show three different fields of view of photobleaching curves that were measured over time. FIG. 5A=Comparator Compound A. FIG. 5B=Compound 41. FIG. 5C=Compound 51. FIG. 5D=Compound 47.

FIG. 6 shows a spectral profile for live and dead cells labeled with Comparator Compound A.

FIG. 7 shows a spectral profile for live and dead cells labeled with Compound B (Compound 41).

FIG. 8 shows a spectral profile for live and dead cells labeled with Compound C (Compound 47).

FIG. 9 shows a spectral profile for live and dead cells labeled with Compound D (Compound 51).

FIG. 10A-10D show fluorescence histogram data for Compounds A-D at 720/24 nm emission with excitation at 640 nm. FIG. 10A=Comparator Compound A. FIG. 10B=Compound B. FIG. 10C=Compound C. FIG. 10D=Compound D.

FIG. 11A-11D show fluorescence histogram data for Compounds A-D at 760/50 nm emission with excitation at 640 nm. FIG. 11A=Comparator Compound A. FIG. 11B=Compound B. FIG. 11C=Compound C. FIG. 11D=Compound D.

FIG. 12A-12D show fluorescence histogram data for Compounds A-D at 770LP nm emission with excitation at 640 nm. FIG. 12A=Comparator Compound A. FIG. 12B=Compound B. FIG. 12C=Compound C. FIG. 12D=Compound D.

FIG. 13 is an example of a graphical user interface that may be used in the performance of computational methods disclosed herein, in accordance with various embodiments.

FIG. 14 is a block diagram of an example computing device that may perform some or all of the computational methods disclosed herein, in accordance with various embodiments.

FIG. 15 is a block diagram of an example system in which some or all of the computational methods disclosed herein may be performed, in accordance with various embodiments.

### DETAILED DESCRIPTION

Reference will now be made in detail to certain embodiments, examples of which are illustrated in the accompanying drawings. While the disclosure provides illustrated embodiments, it will be understood that they are not intended to limit the present disclosure to those embodiments. On the contrary, the present disclosure is intended to cover all alternatives, modifications, and equivalents, which may be included within the disclosure as defined by the appended claims.

Any section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

Unless otherwise stated, the following terms used in the specification and claims are defined for the purposes of this disclosure and have the following meaning:

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, and the like. As used herein, the term “alkylene” refers to a straight or branched, saturated, aliphatic diradical having the number of carbon atoms indicated. For example, C₁-C₆ alkyl includes, but is not limited to, methylene, ethylene, propylene, butylene, pentylene, hexylene, and the like. It will be appreciated that alkyl and alkylene groups can be optionally substituted with one or more substituents by replacement of one or more hydrogen atoms on the alkyl and alkylene group.

As used herein, the term “alkenyl” refers to either a straight chain or branched hydrocarbon radical having the number of carbon atoms indicated, and having at least one double bond. For example, C₂-C₆ alkenyl, includes, but is not limited to, vinyl, propenyl, isopropenyl, butenyl, isobutenyl, butadienyl, pentenyl, hexadienyl, and the like. As used herein, the term “alkenylene” refers to either a straight chain or branched hydrocarbon diradical having the number of carbon atoms indicated, having at least one double bond. For example, C₂-C₆ alkenyl, includes, but is not limited to, vinyl, propenyl, isopropenyl, butenyl, isobutenyl, butadienyl, pentenyl, hexadienyl, and the like. It will be appreciated that alkenyl and alkenylene groups can be optionally substituted with one or more substituents by replacement of one or more hydrogen atoms on the alkenyl and alkenylene group.

As used herein, the term “alkoxy” refers to alkyl radical with the inclusion of at least one oxygen atom within the alkyl chain or at the terminus of the alkyl chain, for example, methoxy, ethoxy, and the like. “Halo-substituted-alkoxy” refers to an alkoxy where at least one hydrogen atom is substituted with a halogen atom. For example, halo-substituted-alkoxy includes trifluoromethoxy, and the like. As used herein, the term “oxy-alkylene” refers to alkyl diradical with the inclusion of an oxygen atom, for example, —OCH₂, —OCH₂CH₂—, —OC₁-C₁₀ alkylene-, —C₁-C₆alkylene-O—C₁-C₆ alkylene-, poly(alkylene glycol), poly(ethylene glycol) (or PEG), and the like. “Halo-substituted-oxy-alkylene” refers to an oxy-alkylene where at least one hydrogen atom is substituted with a halogen atom. It will be appreciated that alkoxy and oxy-alkylene groups can be optionally substituted with one or more substituents by replacement of one or more hydrogen atoms on the alkoxy and oxy-alkylene group.

As used herein, the term “alkynyl” refers to either a straight chain or branched hydrocarbon radical having the number of carbon atoms indicated, and having at least one triple bond. For example, C₂-C₆ alkynyl, includes, but is not limited to, acetylenyl, propynyl, butynyl, and the like. As used herein, the term “alkynylene” refers to either a straight chain or branched hydrocarbon diradical having the number of carbon atoms indicated, and having at least one triple bond. Examples of alkynylene groups include, but are not limited to, —C≡C—, —C≡CCH₂—, —C≡CCH₂CH₂—, —CH₂C≡CCH₂—, and the like. It will be appreciated that alkynyl and alkynylene groups can be optionally substituted with one or more substituents by replacement of one or more hydrogen atoms on the alkynyl and alkynylene group.

As used herein, the term “aryl” refers to a cyclic hydrocarbon radical having the number of carbon atoms indicated, and having a fully conjugated π-electron system. For example, C₆-C₁₀ aryl, includes, but is not limited to, phenyl, naphthyl, and the like. As used herein, the term “arylene” refers to a cyclic hydrocarbon diradical having the number of carbon atoms indicated, and having a fully conjugated π-electron system. For example, C₆-C₁₀ arylene, includes, but is not limited to, phenylene, naphthylene, and the like. It will be appreciated that aryl and arylene groups can be optionally substituted with one or more substituents by replacement of one or more hydrogen atoms on the aryl and arylene group.

“Heteroalkyl,” Heteroalkanyl,” Heteroalkenyl,” Heteroalkynyl,” Heteroalkyldiyl” and “Heteroalkylene,” by themselves or as part of another substituent, refer to alkyl, alkanyl, alkenyl, alkynyl, alkyldiyl and alkylene groups, respectively, in which one or more of the carbon atoms are each independently replaced with the same or different heteroatoms or heteroatomic groups. Typical heteroatoms and/or heteroatomic groups which can replace the carbon atoms include, but are not limited to, —O—, —S—, —S—O—, —NR′—, —PH—, —S(O)—, —SO₂—, —S(O)NR′—, —SO₂NR′—, and the like, including combinations thereof, where R′ is hydrogen or a substitutents, such as, for example, (C₁-C₈) alkyl, (C₆-C₁₄) aryl or (C₇-C₂₀) arylalkyl.

“Cycloalkyl” and “Heterocycloalkyl,” by themselves or as part of another substituent, refer to cyclic versions of “alkyl” and “heteroalkyl” groups, respectively. For heteroalkyl groups, a heteroatom can occupy the position that is attached to the remainder of the molecule. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Typical heterocycloalkyl groups include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, tetrahydronaphthalene, triphenylene, trinaphthalene, and the like.

“Arylalkyl,” by itself or as part of another substituent, refers to an acyclic alkyl group in which one of the hydrogen atoms bonded to a carbon atom, in some embodiments a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where alkyl moieties having a specified degree of saturation are intended, the nomenclature arylalkanyl, arylalkenyl and/or arylalkynyl is used. When a defined number of carbon atoms are stated, for example, (C₇-C₂₀) arylalkyl, the number refers to the total number of carbon atoms comprising the arylalkyl group.

“Parent Heteroaromatic Ring System” refers to a parent aromatic ring system in which one or more carbon atoms are each independently replaced with the same or different heteroatoms or heteroatomic groups. Typical heteroatoms or heteroatomic groups to replace the carbon atoms include, but are not limited to, N, NH, P, O, S, S(O), SO₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include common substituents, such as, for example, benzopyrone and 1-methyl-1,2,3,4-tetrazole. Typical parent heteroaromatic ring systems include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl,” by itself or as part of another substituent, refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Typical heteroaryl groups include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof.

“Heteroarylalkyl,” by itself or as part of another substituent, refers to an acyclic alkyl group in which one of the hydrogen atoms bonded to a carbon atom, in some embodiments a terminal or sp³ carbon atom, is replaced with a heteroaryl group. Where alkyl moieties having a specified degree of saturation are intended, the nomenclature heteroarylalkanyl, heteroarylalkenyl and/or heteroarylalkynyl is used. When a defined number of atoms are stated, for example, 6-20-membered hetoerarylalkyl, the number refers to the total number of atoms comprising the arylalkyl group.

“Haloalkyl,” by itself or as part of another substituent, refers to an alkyl group in which one or more of the hydrogen atoms is replaced with a halogen. Thus, the term “haloalkyl” is meant to include monohaloalkyls, dihaloalkyls, trihaloalkyls, etc. up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

As used here, the term “sulfo” refers to a sulfonic acid, or salt of sulfonic acid (sulfonate).

As used here, the term “carboxy” refers to a carboxylic acid or salt of carboxylic acid.

As used here, the term “phosphate,” refers to an ester of phosphoric acid, and includes salts of phosphate.

As used here, the term “phosphonate,” refers to a phosphonic acid and includes salts of phosphonate.

As used herein, unless otherwise specified, the alkyl portions of substituents such as alkyl, alkoxy, arylalkyl, alkylamino, dialkylamino, trialkylammonium, or perfluoroalkyl are optionally saturated, unsaturated, linear or branched, and all alkyl, alkoxy, alkylamino, and dialkylamino substituents may be optionally substituted by carboxy, sulfo, amino, or hydroxy.

As used herein, “substituted” refers to a molecule wherein one or more hydrogen atoms are replaced with one or more non-hydrogen atoms, functional groups or moieties. Exemplary substituents include but are not limited to halogen, e.g., fluorine and chlorine, C₁-C₈ alkyl, C₆-C₁₄ aryl, heterocycle, sulfate, sulfonate, sulfone, amino, ammonium, amido, nitrile, nitro, lower alkoxy, phenoxy, aromatic, phenyl, polycyclic aromatic, heterocycle, water-solubilizing group, linkage, and linking moiety. In some embodiments, substituents include, but are not limited to, —X, —R, —OH, —OR, —SR, —SH, —NH₂, —NHR, —NR₂, —NR₃+, —N═NR₂, —CX₃, —CN, —OCN, —SCN, —NCO, —NCS, —NO, —NO₂, -N₂+, —N₃, —NHC(O)R, —C(O)R, —C(O)NR₂, —S(O)₂O—, —S(O)₂R, —OS(O)₂OR, —S(O)₂NR, —S(O)R, —OP(O)(OR)₂, —P(O)(OR)₂, —P(O)(O⁻)₂, —P(O)(OH)₂, —C(O)R, —C(O)X, —C(S)R, —C(O)OR, —CO₂—, —C(S)OR, —C(O)SR, —C(S)SR, —C(O)NR₂, —C(S)NR₂, —C(NR)NR₂, where each X is independently a halogen and each R is independently —H, C₁-C₆ alkyl, C₆-C₁₄ aryl, heterocycle, or linking group.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As used herein, the terms “azaindole” and “pyrrolopyridine” are used interchangeably to refer to a heterocyclic aromatic organic compound having a bicyclic structure that includes a pyrrole ring fused to a pyridine ring.

As used herein, the terms “azaindole cyanine” and “pyrrolopyridine cyanine” are used interchangeably to refer to a cyanine compound that includes at least one azaindole group. Certain azaindole cyanine compounds disclosed herein can include one or two optionally substituted azaindole groups. For compounds including two azaindole groups, the azaindole groups can be the same or different.

The compounds disclosed herein may exist in unsolvated forms as well as solvated forms, including hydrated forms. In some embodiments, the compounds disclosed herein are soluble in an aqueous medium (e.g., water or a buffer). For example, the compounds can include substituents (e.g., water-solubilizing groups) that render the compound soluble in the aqueous medium. Compounds that are soluble in an aqueous medium are referred to herein as “water-soluble” compounds. Such water-soluble compounds are particularly useful in biological assays. These compounds may exist in multiple crystalline or amorphous forms. In general, all physical forms are equivalent for the uses described herein and are intended to be within the scope of the present disclosure. The compounds disclosed herein may possess asymmetric carbon atoms (i.e., chiral centers) or double bonds; the racemates, diastereomers, geometric isomers and individual isomers of the compounds described herein are within the scope of the present disclosure. The compounds described herein may be prepared as a single isomer or as a mixture of isomers.

Where substituent groups are specified by their conventional chemical formulae and are written from left to right, they equally encompass the chemically identical substituents, which would result from writing the structure from right to left, e.g., —CH₂O— will be understood to also recite —OCH₂—.

Many embodiments of the compounds of the invention possess an overall electronic charge. In addition, it will also be apparent that the compounds may exist in many different protonation states, depending on, among other things, the pH of their environment. While the structural formulae provided herein depict the compounds in only one of several possible protonation states, it will be understood that these structures are illustrative only, and that the disclosure is not limited to any particular protonation state. Any and all protonated forms of the compounds are intended to fall within the scope of the disclosure.

Furthermore, the compounds of the disclosure may bear multiple positive or negative charges. It is to be understood that when such electronic charges are shown to be present, they are balanced by the presence of an appropriate counterion, which may or may not be explicitly identified. A biologically compatible counterion, which is preferred for some applications, is not toxic in biological applications, and does not have a substantially deleterious effect on biomolecules. Where the compound of the invention is positively charged, the counterion is typically selected from, but not limited to, chloride, bromide, iodide, sulfate, alkanesulfonate, arylsulfonate, phosphate, perchlorate, tetrafluoroborate, tetraarylboride, nitrate and anions of aromatic or aliphatic carboxylic acids. Where the compound of the invention is negatively charged, the counterion is typically selected from, but limited to, alkali metal ions, alkaline earth metal ions, transition metal ions, ammonium or substituted ammonium or pyridinium ions. Preferably, any necessary counterion is biologically compatible, is not toxic as used, and does not have a substantially deleterious effect on biomolecules. Counterions are readily changed by methods well known in the art, such as ion-exchange chromatography, or selective precipitation. It will be understood that the identity of any associated counter ion is not a critical feature of the disclosure, and that the disclosure encompasses the compounds in association with any type of counter ion unless otherwise specified.

It will be understood that the chemical structures that are used to define the compounds disclosed herein are each representations of one of the possible resonance structures by which each given structure can be represented. Further, it will be understood that, by definition, resonance structures are merely a graphical representation used by those of skill in the art to represent electron delocalization, and that the present disclosure is not limited in any way by showing one particular resonance structure for any given structure.

Where a disclosed compound includes a conjugated ring system, resonance stabilization may permit a formal electronic charge to be distributed over the entire molecule. While a particular charge may be depicted as localized on a particular ring system, or a particular heteroatom, it is commonly understood that a comparable resonance structure can be drawn in which the charge may be formally localized on an alternative portion of the compound.

The above-defined groups may include prefixes and/or suffixes that are commonly used in the art to create additional well-recognized substituent groups. As non-limiting specific examples, “alkyloxy” and/or “alkoxy” refer to a group of the formula —OR″, “alkylamine” refers to a group of the formula —NHR″ and “dialkylamine” refers to a group of the formula —NR″R″, where each R″ is an alkyl.

The term “detectable response” as used herein refers to an occurrence of or a change in, a signal that is directly or indirectly detectable either by observation or by instrumentation. In some embodiments, the detectable response is an optical response resulting in a change in the wavelength distribution patterns or intensity of absorbance or fluorescence or a change in light scatter, fluorescence lifetime, fluorescence polarization, or a combination of the above parameters.

As used herein, the term “staining” is a technique used in microscopy to enhance contrast in the microscopic image. Stains and dyes are frequently used to highlight structures in biological tissues and cells. Staining also involves adding a dye to a substrate to quantify or qualify the presence of a specific compound, such as a protein, nucleic acid, lipid or carbohydrate. Biological staining is also used to mark cells in flow cytometry and to flag proteins or nucleic acids in gel electrophoresis. Staining is not limited to biological materials and can be used to study the morphology of other materials such as semi-crystalline polymers and block copolymers.

The present disclosure relates to compounds chosen from Formula (I) or Formula (II):

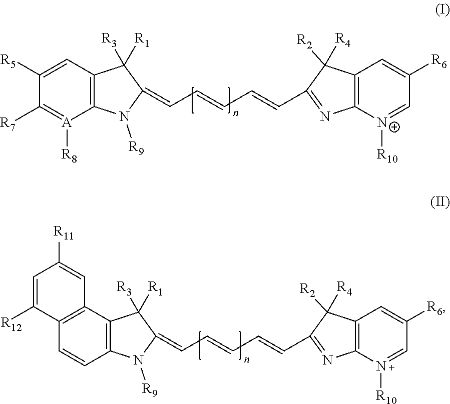

wherein
A is C or N;
n is 0, 1, or 2;
each of R₁, R₂, R₃, and R₄ is the same or different and is independently chosen from alkyl, heteroalkyl, sulfoalkyl group, alkyl with terminal -Su (succinimidyl/sulfosuccinimidyl) ester, heteroalkyl with terminal —SO₃, and alkyl with terminal (Q)_{z}C(O)OR*, and heteroalkyl with terminal (Q)_{z}C(O)OR*,
   where Q is a 5 or 6 membered aryl or heteroaryl ring, z is 0, 1, 2, or 3, R* is chosen from —H, —(CH₂)_{y}SO₃, and N-hydroxysuccinimide, and y is 1, 2, 3, 4, or 5;
each of R₅, R₆, R₁₁, and R₁₂ is the same or different and is independently chosen from —SO₃, -lower alkylene-QR₁₃, -Q(CH₂)_{z}R₁₃, -(Q′)_{y}(R₁₃)_{z}, and -Q″(R₁₃),
   where Q′ is chosen from thiophene, furan, and pyrrole rings, Q″ is a dithienothiophene, and R₁₃ is chosen from —H, —SO₃, —COOH;
R₇ is —H or SO₃;
R₈ is chosen from —H, alkyl, and L^{a}-Z;
or alternatively, R₇ is SRr, and R₈ is alkyl, wherein Rr is an alkylene that forms an unsaturated ring with R₈;
R₉ is chosen from —H, alkyl, sulfoalkyl, L^{a}-Z, and L^{a}-X, on the condition that R₈ and R₉ are not both —H; and
R₁₀ is chosen from alkyl, sulfoalkyl, L^{a}-Z, and L^{a}-X,
   where L^{a} is selected from the group consisting of a divalent linear (—(CH₂)ₒ—, o=0 to 15), crossed, or cyclic alkane group that can be substituted by at least one atom selected from substituted nitrogen and/or sulfur;
   where L is selected from the group consisting of a divalent linear (—(CH₂)ₒ—, o=0 to 15), crossed, or cyclic alkane group that can be substituted by at least one atom selected from the group consisting of oxygen, substituted nitrogen, and/or sulfur;
   where Z is selected from the group consisting of H, CH₃, alkyl, heteroalkyl, NH₂, —COO—, —COOH, —COSH, CO—NH—NH₂, —COF, —COCl, —COBr, —COI, —COO-Su (succinimidyl/sulfosuccinimidyl) ester, —COO-STP (4-sulfo-2,3,5,6-tetrafluorophenyl), —COO-TFP (2,3,5,6-tetrafluorophenyl), —COO-benzotriazole, —CO-benzotriazole, —CONR′—CO—CH₂—I, —CONR′R″, —CONR′-biomolecule, —CONR′-L-COO—, —CONR′-L-COOH, —CONR′-L-COO-Su, —CONR′-L-COO-STP, —CONR′-L-COO-TFP, —CONR′-L-CONR″₂, —CONR′-L-CO-biomolecule, —CONR′-L-CO—NH—NH₂, —CONR′-L-OH, —CONR′-L-CHO, —CONR′-L-maleimide, and —CONR′-L-NH—CO—CH₂—I; R′ and R″ is selected from the group consisting of H, alkyl group, and heteroalkyl group, and the biomolecule is a protein, antibody, nucleotide, oligonucleotide, biotin, or hapten;
   X is selected from the group consisting of —SH, —NH₂, —NH—NH₂, —F, —Cl, —Br, I, —NHS (hydroxysuccinimidyl/sulfosuccinimidyl) ester, —O-TFP (2,3,5,6-tetrafluorophenoxy), —O-STP (4-sulfo-2,3,5,6-tetrafluorophenoxy), —O-benzotriazole, -benzotriazole, —NR-L-OH, —NR-L-SH, —NR-L-NH₂, —NR-L-NH—NH₂, —NR-L-CO₂H, —NR-L-CO—NHS, —NR-L-CO-STP, —NR-L-CO-TFP, —NR-L-CO-benzotriazole, —NR-L-CHO, —NR-L-maleimide, and —NR-L-NH—CO—CH2-I, where R is —H or an alkyl or heteroalkyl group; and with the proviso that the compound of Formula (II) cannot be chosen from:

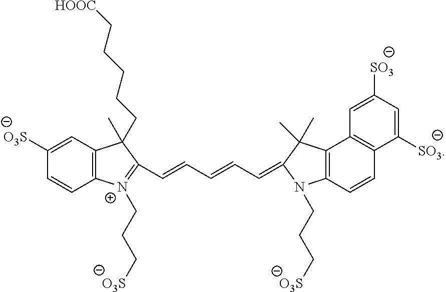

In some embodiments of the present disclosure, the compound is chosen from Formula (I) wherein A is N. In some embodiments of the present disclosure, the compound is chosen from Formula (I), wherein A is C.

In some embodiments of the present disclosure, the compound is chosen from Formula (I) wherein R₁, R₂, R₃, and R₄ are each methyl. In some embodiments of the present disclosure, the compound is chosen from Formula (I) wherein one of R₁ or R₃ is alkyl with terminal (Q)_{z}C(O)OR*.

In some embodiments of the present disclosure, the compound is chosen from Formula (I) wherein each of R₅ and R₆ is independently chosen from -Q(CH₂)_{z}R₁₃. In some embodiments of the present disclosure, the compound is chosen from Formula (I) wherein one of R₅ and R₆ is —SO₃ and the other is -Q(CH₂)_{z}R₁₃. In some embodiments of the present disclosure, the compound is chosen from Formula (I) wherein one of R₅ and R₆ is -lower alkylene-QR₁₃ and the other is -Q(CH₂)_{z}R₁₃.

In some embodiments of the present disclosure, the compound is chosen from Formula (I) wherein R₇ is —H. In some embodiments of the present disclosure, the compound is chosen from Formula (I) wherein R₇ is SRr, and R₈ is alkyl, wherein Rr is an alkylene that forms an unsaturated ring with R₈. In some embodiments of the present disclosure, the compound is chosen from Formula (I) wherein R₈ is —H. In some embodiments of the present disclosure, the compound is chosen from Formula (I) wherein R₈ is L^{a}-Z.

In some embodiments of the present disclosure, the compound is chosen from Formula (I) wherein R₉ is chosen from —H. In some embodiments of the present disclosure, the compound is chosen from Formula (I) wherein R₉ is chosen from —H. In some embodiments of the present disclosure, the compound is chosen from Formula (I) wherein R₉ is chosen from L^{a}-Z and L^{a}-X.

In some embodiments of the present disclosure, the compound is chosen from Formula (I) wherein R₁₀ is chosen from sulfoalkyl. In some embodiments of the present disclosure, the compound is chosen from Formula (I) wherein R₁₀ is chosen from L^{a}-Z and L^{a}-X.

In some embodiments of the present disclosure, the compound is chosen from Formula (II), wherein at least one of R₁ and R₃ is chosen from alkyl with terminal -Su (succinimidyl/sulfosuccinimidyl) ester. In some embodiments of the present disclosure, the compound is chosen from Formula (II) wherein at least two of R₁, R₂, R₃, and R₄ are independently chosen from alkyl. In some embodiments of the present disclosure, the compound is chosen from Formula (II) at least three of R₁, R₂, R₃, and R₄ are independently chosen from alkyl. In some embodiments of the present disclosure, the compound is chosen from Formula (II) wherein at least one of R₁ and R₃ is chosen from alkyl with terminal (Q)_{z}C(O)OR*. In some embodiments of the present disclosure, the compound is chosen from Formula (II) wherein at least one of R₁ and R₃ is chosen from heteroalkyl with terminal —SO₃.

In some embodiments of the present disclosure, the compound is chosen from Formula (II) wherein R₆ is -Q(CH₂)_{z}R₁₃. In some embodiments of the present disclosure, the compound is chosen from Formula (II) wherein R₆, is -(Q′)_{y}(R₁₃)_{z}. In some embodiments of the present disclosure, the compound is chosen from Formula (II) wherein R₆ is -lower alkylene-QR₁₃. In some embodiments of the present disclosure, the compound is chosen from Formula (II) wherein R₆ is -Q″(R₁₃).

In some embodiments of the present disclosure, the compound is chosen from Formula (II) wherein R₉ is sulfoalkyl. In some embodiments of the present disclosure, the compound is chosen from Formula (II) wherein R₉ is chosen from L^{a}-Z and L^{a}-X.

In some embodiments of the present disclosure, the compound is chosen from Formula (II) wherein R₁₀ is chosen from alkyl, sulfoalkyl, L^{a}-Z, and L^{a}-X. In some embodiments of the present disclosure, the compound is chosen from Formula (II) wherein R₁₀ is chosen from sulfoalkyl. In some embodiments of the present disclosure, the compound is chosen from Formula (II) wherein R₁₀ is chosen from L^{a}-Z and L^{a}-X.

In some embodiments of the present disclosure, the compound is chosen from Formula (II) wherein at least one of R₁₁ and R₁₂ is —SO₃. In some embodiments of the present disclosure, the compound is chosen from Formula (II) wherein each of R₁ and R₁₂ is —SO₃.

In at least one embodiment of the present disclosure, the compound is of Formula (I) as described herein wherein n is 1.

In at least one embodiment of the present disclosure, the compound is of Formula (II) as described herein wherein n is 1.

As noted, the azaindole cyanine compounds of the present disclosure may optionally possess a linking group (LG) comprising at least one group -L^{a}-Z or -L^{a}-X, where Z and X is a reactive group that is attached to the dye D by a covalent linkage L₁. In certain embodiments L₁ comprises multiple intervening atoms that serve as a spacer, while in other embodiments L₁ is simply a bond linking Z or X to the dye. Dyes having a linking group may be reacted with a wide variety of organic or inorganic substances Sc that contain or are modified to contain functional groups with suitable reactivity, i.e., a complementary functionality -L₂-R_{z} or -L₂Rₓ. In certain embodiments L₂ comprises multiple intervening atoms that serve as a spacer, while in other embodiments L₂ is simply a bond linking R_{z} or Rₓ to the substance Sc. Reaction of the linking group and the complementary functionality results in chemical attachment of the dye to the conjugated substance Sc, represented by D-L-Sc, where L is the linkage formed by the reaction of the linking group and the complementary functionality.

One of R_{y} or Rₓ/R_{z} typically comprise an electrophile, while the other typically comprises a nucleophile, such that the reaction of the electrophile and nucleophile generate a covalent linkage between the dye and the conjugated substance.

Alternatively, one of R_{y} or R/R_{z} typically comprise a photoactivatable group, and becomes chemically reactive only after illumination with light of an appropriate wavelength.

Selected examples of electrophiles and nucleophile that are useful in linking groups and complementary functionalities are shown in Table 1, where the reaction of an electrophilic group and a nucleophilic group yields a covalent linkage.

TABLE 1
Examples of routes to covalent linkages

| Electrophilic | Nucleophilic | Resulting |
|---|---|---|
| Group | Group | Covalent Linkage |
| activated esters* | amines/anilines | carboxamides |
| aldehydes | amines/anilines | imines |
| aldehydes or ketones | hydrazines | hydrazones |
| aldehydes or ketones | hydroxylamines | oximes |
| anhydrides | alcohols/phenols | esters |
| anhydrides | amines/anilines | carboxamides |
| aziridines | thiols | thioethers |
| boronates | glycols | boronate esters |
| carbodiimides | carboxylic acids | N-acylureas or anhydrides |
| epoxides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | alcohols | ethers |
| sulfonyl halides | amines/anilines | sulfonamides |
| sulfonyl halides | phenols/alcohols | sulfonate esters |
| azide | alkyne | 1,2,3-triazole |

*Activated esters, as understood in the art, generally have the formula —COΩ, where Ω is a good leaving group (e.g. oxysuccinimidyl (—ONC₄H₄O₂) oxysulfosuccinimidyl (—ONC₄H₃O₂—SO₃H), 1-oxybenzotriazoyl (—OC₆H₄N₃); or an aryloxy group or aryloxy substituted one or more times by electron withdrawing substituents such as nitro, fluoro, chloro, cyano, or trifluoromethyl, or combinations thereof, used to form an anhydride or mixed anhydride —OCORª or —OCNRªNHR^{b}, where R^{a} and R^{b}, whichmay be the same or different, are C1-C6 alkyl, C1-C6 perfluoroalkyl, or C1-C6 alkoxy; or cyclohexyl, 3-dimethylaminopropyl, or N-morpholinoethyl
**Acyl azides can also rearrange to isocyanates.

In some embodiments of the compounds of Formula (I) are selected from those of Table A:

TABLE A
Representative Azaindole Cyanine Dye Compounds

| Com- | | Ex/ |
|---|---|---|
| pound | | Em |
| # | Structure | (nm) |
| 18 | 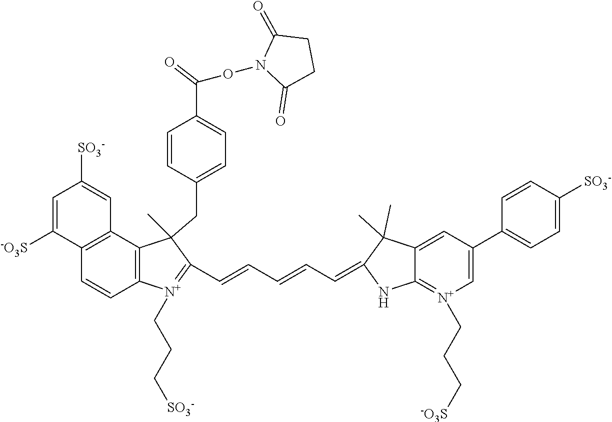 | 700/ 724 |
| 30 | 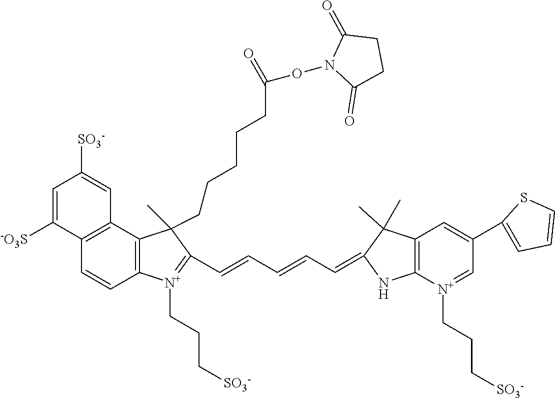 | 707/ 732 |
| 36 | 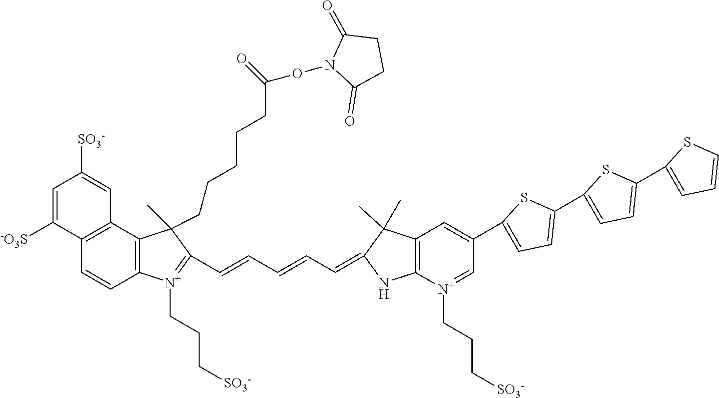 | 725/ 747 |
| 41 | 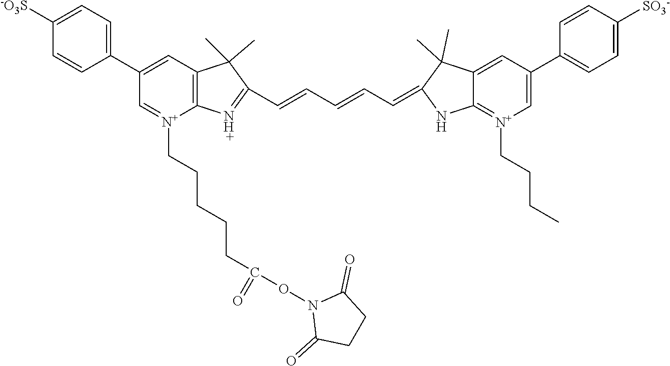 | 721/ 743 |
| 47 | 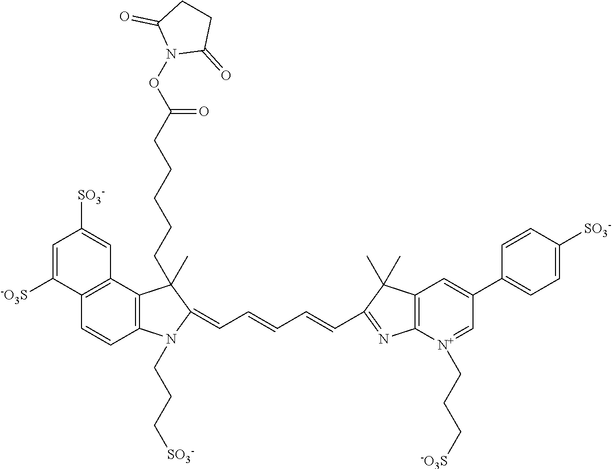 | 699/ 722 |
| 51 | 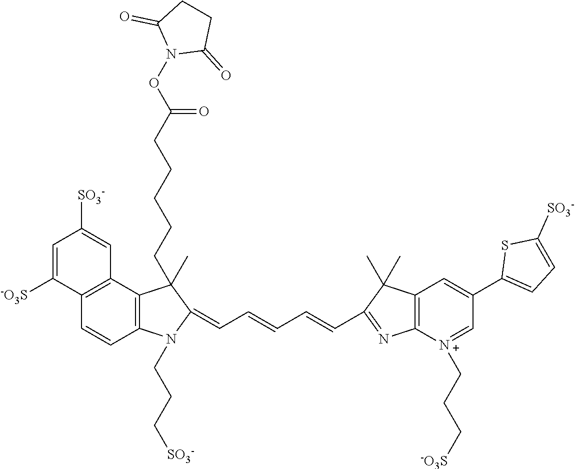 | 705/ 732 |
| 56 | 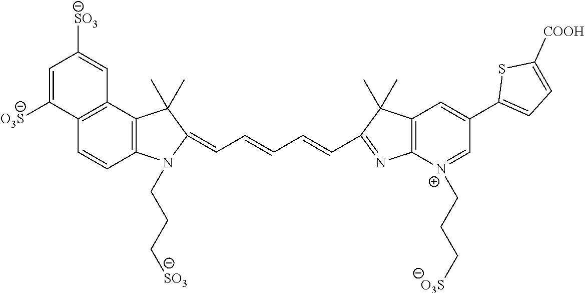 | 705/ 732 |
| 61 | 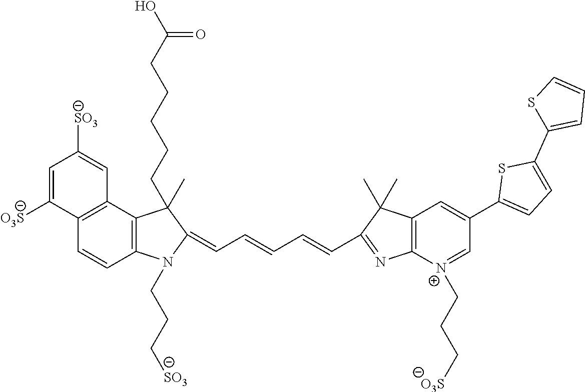 | 711/ 742 |
| 66 | 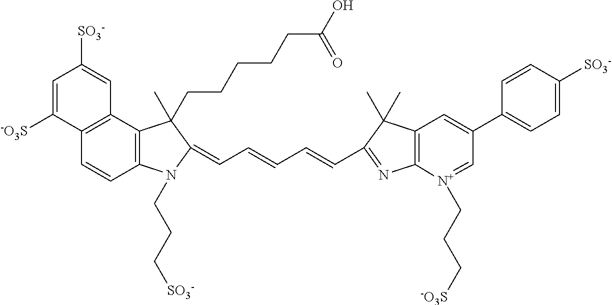 | 707/ 741 |
| 72 | 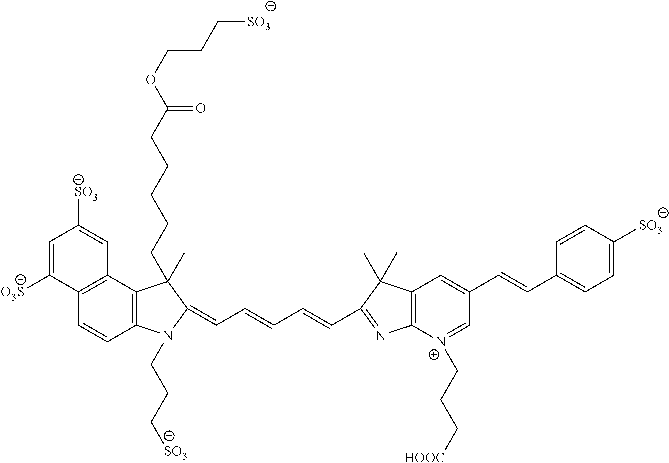 | 712/ 740 |
| 79 | 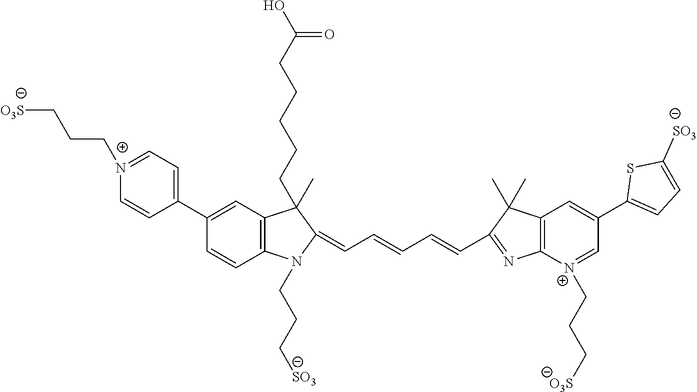 | 702/ 732 |
| 81 | 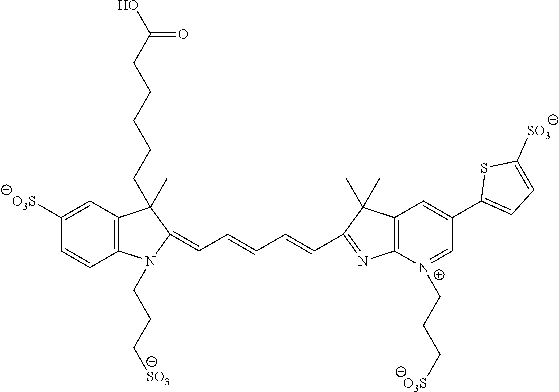 | 685/ 711 |
| 86 | 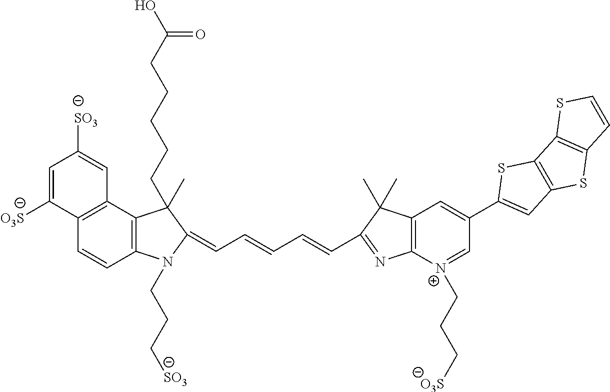 | 713/ 742 |
| 91 | 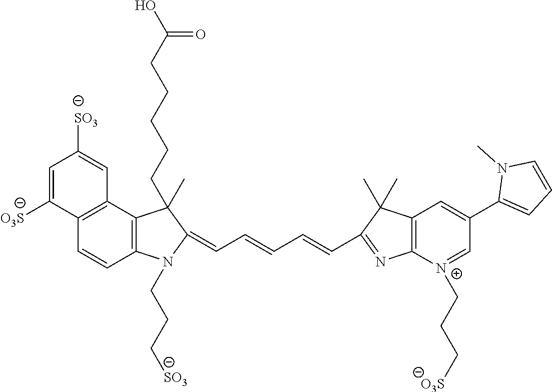 | 696/ 726 |
| 92 | 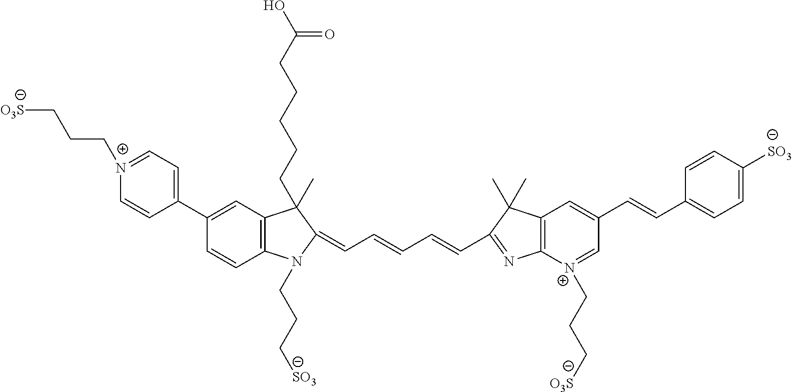 | 707/ 741 |
| 96 | 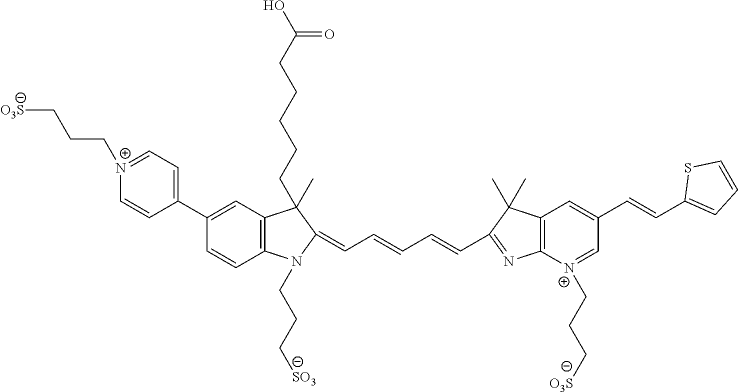 | 709/ 741 |
| 104 | 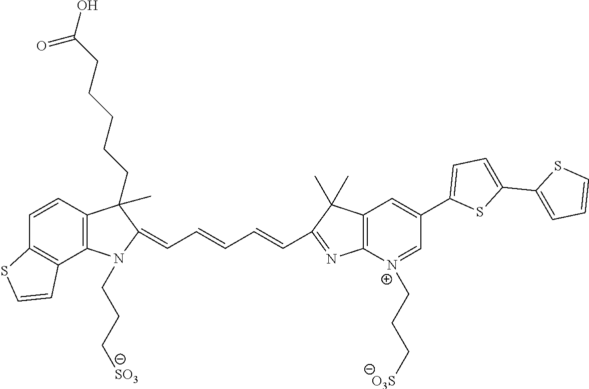 | 723/ 760 |
| 110 | 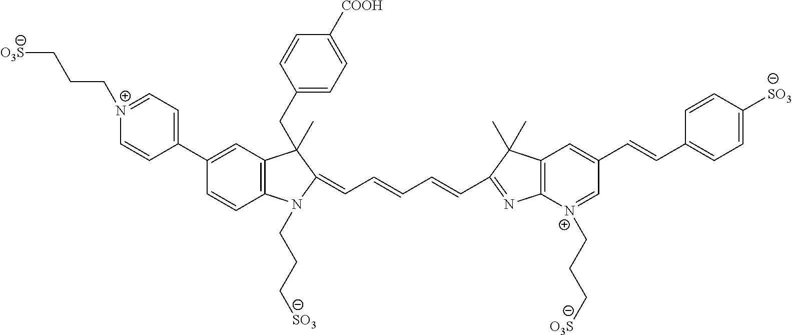 | 708/ 742 |
| 111 | 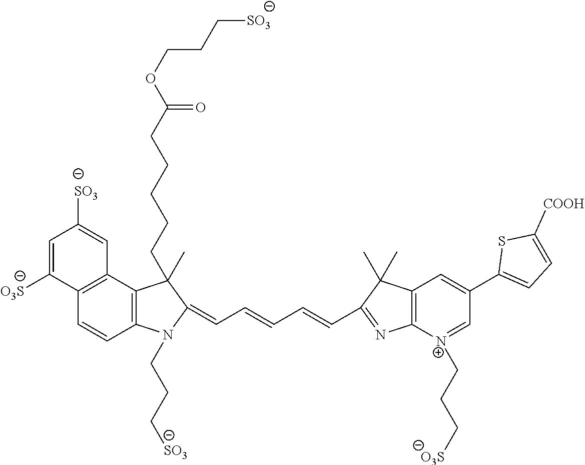 | 705/ 732 |

The present disclosure also relates to methods of detecting at least one biomolecule, the method comprising combining at least one biomolecule with a composition comprising at least one excipient and a compound of Formula (I) or Formula (II) as described herein in an effective concentration to label at least one biomolecule under conditions sufficient for binding the compound to the biomolecule, and detecting the biomolecule-bound compound. In some embodiments of the present disclosure, the biomolecule is selected from a protein, antibody, enzyme, nucleoside triphosphate, oligonucleotide, biotin, hapten, cofactor, lectin, antibody binding protein, carotenoid, carbohydrate, hormone, neurotransmitter, growth factors, toxin, biological cell, lipid, receptor binding drug, fluorescent proteins, organic polymer carrier material, inorganic polymeric carrier material, and combinations thereof. In some embodiments of the present disclosure, the at least one biomolecule is detected in an assay selected from fluorescence microscopy, flow cytometry, in vivo imaging, immunoassay, hybridization, chromatographic assay, electrophoretic assay, microwell plate based assay, fluorescence resonance energy transfer (FRET) system, bioluminescence resonance energy transfer (BRET) system, high throughput screening, or microarray. In some embodiments of the present disclosure, the biomolecule is detected by in vivo imaging comprising providing the biomolecule-bound compound to at least one of a biological sample, tissue, or organism, and detecting the biomolecule within the at least one of a biological sample, tissue, or organism.

The compounds and conjugates provided herein can be used in a variety of biological applications. In many fields it is useful or necessary to detect or quantify biological material using fluorescent dyes. Compounds and/or conjugates disclosed herein can be used, e.g., in optical, including fluorescence optical, qualitative and/or quantitative determination methods to diagnose properties of cells (molecular imaging), in biosensors (point of care measurements), for investigation of the genome, and in miniaturizing technologies. Microscopy, super-resolution imaging, cytometry, cell sorting, fluorescence correlation spectroscopy (FCS), ultra-high throughput screening (uHTS), multicolor fluorescence in situ hybridization (mc-FISH), FRET-systems, BRET-systems, and microarrays (DNA- and protein-chips) are exemplary application fields. Biological materials such as nucleic acids, proteins, polypeptides, cells, and membranes can be detected in various sample types, e.g., in biomedical, genetic, fermentation, aquaculture, agricultural, forensic and environmental applications.

The compounds provided herein can be used to label or stain cells, cellular structures, organelles, target molecules, nucleic acid molecules, and the like. To provide a dye with the desired properties for a particular application, it is often useful or necessary to functionalize or conjugate the dye to a moiety that targets the dye to particular components, such as a given intracellular location, intracellular component (e.g., cytoskeletal component or nucleic acid), or epitope or to a nucleic acid (e.g., an oligonucleotide).

In some embodiments, methods of staining or labeling cells or cellular structures are provided, the methods comprising: a) contacting a sample containing one or more cells or cellular structures with a compound of Formula (I) to form a contacted sample; b) incubating the contacted sample for an appropriate amount of time to form an incubated sample; c) illuminating the sample with an appropriate wavelength to form an illuminated sample; and d) detecting fluorescence emission from the illuminated sample.

Also provided are methods of detecting target molecules, the methods comprising: a) contacting a sample containing or thought to contain a target molecule with a compound of Formula (I) to form a contacted sample; b) incubating the contacted sample for an appropriate amount of time to form an incubated sample; c) illuminating the sample with an appropriate wavelength to form an illuminated sample; and d) detecting fluorescence emission from the illuminated sample, wherein the fluorescence emission is used to detect the target molecule.

In some embodiments, methods are provided for detecting a biological structure, the methods comprising: a) combining a sample that contains or is thought to contain a specific biological structure, with a compound of Formula (I), wherein said biological structure contains nucleic acids; b) incubating the combined sample and the compound for a time sufficient for the compound to combine with the nucleic acids in the biological structure to form a pattern of the -nucleic acid complexes having a detectable fluorescent signal that corresponds to the biological structure; and c) detecting the fluorescent signal that corresponds to the biological structure.

In some embodiments, compounds provided herein can be conjugated to antibodies as described herein to provide compounds of Formula (I) comprising an antibody. As such, methods for staining or detecting an antigen or cells, cellular components, tissues, etc., comprising an antigen are provided, the methods comprising contacting a sample suspected of comprising the antigen with a compound of Formula (I) comprising an antibody specific for the antigen. Also provided herein are methods for staining or detecting an antigen or cells, cellular components, tissues, etc., comprising an antigen are provided, the methods comprising contacting a sample suspected of comprising the antigen with a primary antibody specific for the antigen and contacting the primary antibody with a compound of Formula (I) comprising an antibody specific for the primary antibody (i.e., a secondary antibody labeled with a moiety disclosed herein). Corresponding uses of such compounds of Formula (I) as primary or secondary antibodies to stain or detect an antigen of interest or cells, cellular components, tissues, etc., comprising the antigen are also provided. Such methods and uses encompass immunofluorescence, immunohistochemistry, flow cytometry (e.g., FACS), Western blotting, fluorescence ELISA, and any other approach where fluorescently labeled antibodies can be used.

Certain embodiments provide a method, use, or composition for staining cells and/or assessing cell viability, being compatible for use with, for example, flow cytometry and fluorescence microscopy. Such methods and uses can comprise incubating a cell or mixture of cells with a compound disclosed herein; providing a stimulus to the cell or mixture of cells to elicit a fluorescent signal; and measuring the fluorescent signal. Methods and uses can further comprise quantifying the amount of live and/or dead cells, e.g., by determining whether the degree of fluorescence exceeds a predetermined threshold (indicating nonviability), or by grouping results into live and dead populations (where dead cell populations show a greater extent of staining given their compromised membrane integrity).

The ability to stain live cells with a viability dye and preserve that staining pattern after fixation is critical for certain applications such as, e.g., intracellular immunophenotyping. Exclusion of the dead cells from the data allows cleaner separation and identification of cell populations. The compounds disclosed herein can be utilized as fixable viability dyes that help to ensure accurate assessment of cell viability in samples after fixation and/or permeabilization.

Thus, in some embodiments, the disclosed compounds can be implemented in live/dead cell assays. In some embodiments, the compound of the present disclosure is delivered or passes into nonviable eukaryotic cells, e.g., mammalian cells, such as human cells. When the cells are illuminated with a light source, then fluorescence emissions can be collected, detected, analyzed, or measured. The cells in the assay may be treated with a substance or reagent that induces cell death, e.g., camptothecin or staurosporine. Data may be acquired using any appropriate fluorescence detection apparatus such as a plate reader, fluorescence microscope, or flow cytometer. In certain embodiments, the fluorescence detection can be achieved using an acoustic focusing flow cytometer, such as the ATTUNE N×T Flow Cytometer from Thermo Fisher Scientific. In other embodiments, fluorescence detection can be achieved using a cell sorting flow cytometry instrument, such as the Invitrogen Bigfoot Spectral Cell Sorter from Thermo Fisher Scientific.

In some embodiments, the cell or mixture of cells are incubated with at least one, two, three, or four additional fluorescent molecules, wherein the at least one, two, three, or four additional fluorescent molecules are spectrally distinguishable from the compound according to the present disclosure, and fluorescent signals are measured measuring from the at least one, two, three, or four additional fluorescent molecules. Fluorescence from two sources is considered spectrally distinguishable if the emission maxima are separated by at least 30 nm or if fluorescence from the sources can be distinguished through the use of optical filters, e.g., a pair of filters wherein fluorescence from at least one of the molecules is differentially reduced by at least one of the filters.

In another embodiment, the assay method may be conducted where cells are contained in a plurality of vessels. In any vessel, the cells may be of one type or different types of cells. The cells may be the same type grown under different conditions, e.g. in the presence of different media. Some of the cells may be treated with an apoptosis inducer or a caspase inhibitor. The plurality of vessels, an array, may be illuminated by a light source, e.g. a scanning light source. The cells may be of the same or different organisms.

In some embodiments, the sample containing target material is a cell or is an aqueous or aqueous-miscible solution that is obtained directly from a liquid source or as a wash from a solid material (organic or inorganic) or a growth medium in which cells have been introduced for culturing or a buffer solution in which target material has been placed for evaluation. Where the target material is in cells, the cells are optionally single cells, including microorganisms, or multiple cells associated with other cells in two or three dimensional layers, including multicellular organisms, embryos, tissues, biopsies, filaments, biofilms, etc. Alternatively, the sample is a solid, optionally a smear or scrape or a retentate removed from a liquid or vapor by filtration. In one aspect of the disclosure, the sample is obtained from a biological fluid, including separated or unfiltered biological fluids such as urine, cerebrospinal fluid, blood, lymph fluids, tissue homogenate, interstitial fluid, cell extracts, mucus, saliva, sputum, stool, physiological secretions or other similar fluids. Alternatively, the sample is obtained from an environmental source such as soil, water, or air; or from an industrial source such as taken from a waste stream, a water source, a supply line, or a production lot. Industrial sources also include fermentation media, such as from a biological reactor or food fermentation process such as brewing; or foodstuffs, such as meat, gain, produce, or dairy products.

The azaindole cyanine compounds described herein provide various advantages over other types of fluorescent cyanine compounds that are commonly used in biological applications. In particular, Applicant has found that the presence of one or two azaindole groups attached to the polymethine bridge of the compound can provide several distinct properties that differ from other types of cyanine compounds that operate in the far-red and/or near-IR region of the spectrum. The length of the methine bridge is known to negatively affect the stability of fluorescent cyanine compounds. For example, fluorescent cyanine dyes having longer methine bridges (e.g., 5 carbon bridges or longer), which emit light in the far-red and near-IR spectral region, can be notoriously unstable under the rigorous conditions frequently encountered in certain biological assays. In addition, the brightness of fluorescent cyanine compounds, as measured by quantum yield, is known to degrade as excitation/emission wavelength shifts towards the near-IR region on the spectrum. Applicant has found that incorporation of certain substituted azaindole cyanine compounds, as disclosed herein, can extend the excitation and emission profiles to the far-red or near IR range relative to other types of cyanine compounds known in the art. Further, the azaindole cyanine compounds provided herein can exhibit a higher quantum yield relative to other types of cyanine compounds having equivalent methine bridge length but no azaindole group(s). Azaindole cyanine compounds described herein can have a quantum yield of 15% or greater. In certain embodiments, the compounds can exhibit a quantum yield between about 20%-30%. Certain compounds exhibit a quantum yield of about 20%-22%; or about 22% to about 24%; or about 24% to about 26%; or about 26% to about 28%. The introduction of novel substituted azaindole group(s) into the cyanine dye structure can provide an unexpected combination of exceptional brightness along with excitation and emission profiles that extend well into the near IR spectral region. Further, azaindole cyanine compounds, including those having a 5-membered cyanine bridge, were found to be thermally stable and resist photobleaching even upon prolonged irradiation. By tailoring the type and number of substituents on the azaindole ring(s), novel compounds can be generated that provide a unique combination of fluorescence excitation/emission profiles in the far-red and near-IR, with sustained brightness and water solubility. In particular, the introduction of sulfonate and alkyl sulfonate substituents can greatly enhance the water solubility of the disclosed cyanine compounds. Because the disclosed azaindole cyanines also possess thermal stability and photostability, the described compounds are particularly useful in biological applications involving prolonged heat exposure and/or extended irradiation, such as PCR and fluorescence imaging.

Given that certain compounds disclosed herein have excitation and/or emission profiles in the far-red and/or near-IR region of the electromagnetic spectrum, such compounds are particularly useful for multiplex flow cytometry experiments where bright and stable fluorescent dyes in this region of the spectrum are needed. The compounds described herein provide exceptional brightness, which makes them particularly valuable in exacting flow cytometry applications. The brightness allows for resolution between rare cell populations and antigen targets. Further, due to the unique spectral properties offered by the disclosed dyes, the compounds disclosed herein can greatly expand the number of targets that can be detected in multicolor panels. Because the disclosed compounds operate in the far-red and near-IR spectral region, the disclosed dyes are ideal for use in multicolor panels in both conventional and spectral flow cytometry applications. Further, due to having excitation and/or emission profiles in the far-red and/or near-IR region of the electromagnetic spectrum, the disclosed compounds also are particularly useful for multi-color imaging experiments where bright and stable fluorescent dyes in this region of the spectrum are needed. The compounds described herein provide exceptional photostability and high quantum yield, which makes them particularly valuable in applications requiring extended irradiation (e.g., fluorescence imaging).

In some embodiments, compounds of the present disclosure can be conjugated to a nucleic acid or a nucleic acid binding moiety as described herein to provide compounds of Formula (I) comprising a nucleic acid or nucleic acid binding moiety. In some embodiments, moieties of the present disclosure can be attached to a polynucleotide, or to a nucleotide which is incorporated into a polynucleotide, which can then serve as a labeled primer or probe. Such compounds can be combined with a sample that contains or is thought to contain a nucleic acid polymer, and then the mixture of compound and sample is incubated for a time sufficient for the compound to combine with nucleic acid polymers in the sample to form one or more compound-nucleic acid complexes having a detectable fluorescent signal. The nucleic acid can be DNA, e.g., dsDNA or ssDNA. The nucleic acid can also be RNA or an RNA-DNA hybrid. The compounds can be used to label or detect nucleic acids in a wide variety of samples, such as in aqueous solutions, sequencing or amplification reactions such as PCR, cellular samples (e.g., for FISH or general nuclear or chromosome staining) and electrophoretic gels. Those skilled in the art will recognize when to use compounds comprising general nucleic acid- or DNA-binding moieties and when to use probes or primers that target specific sequences.

In some embodiments, methods of staining nucleic acids are provided, the methods comprising: combining a sample that contains or is thought to contain a nucleic acid with a compound of Formula (I); and incubating the sample and compound for a time sufficient for the compound to combine with the nucleic acid in the sample to form one or more compound-nucleic acid complexes that give a detectable fluorescent signal.

A target polynucleotide can be detected specifically, for example, using a compound described herein which is capable of specifically binding the target polynucleotide, and determining whether a complex comprising the target polynucleotide and the compound was formed, e.g., by detecting fluorescence from the complex or detecting a change in fluorescence resulting from complex formation, optionally wherein the change in fluorescence resulting from complex formation is a reduction of quenching by a quencher due to cleavage or a conformational change.

The characteristics of the compound-nucleic acid complex, including the presence, location, intensity, excitation and emission spectra, fluorescence polarization, fluorescence lifetime, and other physical properties of the fluorescent signal can be used to detect, differentiate, sort, quantitate, and/or analyze aspects or portions of the sample. The compounds of the disclosure are optionally used in conjunction with one or more additional reagents (e.g., detectably different fluorescent reagents), including compounds of the same class having different spectral properties (see, e.g., discussion of shifts in emission and near-infrared dyes elsewhere herein).

As used herein, “energy transfer (ET)” refers to FRET or Dexter energy transfer. As used herein, “FRET” (also referred to as fluorescence resonance energy transfer or Forster resonance energy transfer) refers to a form of molecular energy transfer (MET) by which energy is passed non-radiatively between a donor molecule and an acceptor molecule. Without being bound by theory, it is believed that when two fluorophores whose excitation and emission spectra overlap are in close proximity, excitation of one fluorophore can cause the first fluorophore to transfer its absorbed energy to the second fluorophore, causing the second fluorophore, in turn, to fluoresce. Stated differently, the excited-state energy of the first (donor) fluorophore is transferred by a process sometimes referred to as resonance induced dipole-dipole interaction to the neighboring second (acceptor) fluorophore. As a result, the lifetime of the donor molecule is decreased and its fluorescence is quenched, while the fluorescence intensity of the acceptor molecule is enhanced and depolarized. When the excited-state energy of the donor is transferred to a non-fluorophore acceptor, such as a quencher, the fluorescence of the donor is quenched without subsequent emission of fluorescence by the acceptor. Pairs of molecules that can engage in ET are termed ET pairs. In order for energy transfer to occur, the donor and acceptor molecules must typically be in close proximity (e.g., up to 70 to 100 Angstroms). As used herein, “Dexter energy transfer” refers to a fluorescence quenching mechanism whereby an excitation electron can be transferred from a donor molecule to an acceptor molecule via a non-radiative path. Dexter energy transfer can occur when there is interaction between the donor and acceptor. In some embodiments, the Dexter energy transfer can occur at a distance between the donor and acceptor of about 10 Angstroms or less. In some embodiments, in the Dexter energy transfer, the excited state may be exchanges in a single step. In some embodiments, in the Dexter energy transfer, the excited state mat be exchanges in a two separate steps.

Commonly used methods for detecting nucleic acid amplification products require that the amplified product (i.e., amplicon) be separated from unreacted primers. This is often achieved either through the use of gel electrophoresis, which separates the amplification product from the primers on the basis of a size differential, or through the immobilization of the product, allowing washing away of free primer. Other methods for monitoring the amplification process without separation of the primers from the amplicon, such as for real-time detection, have been described. Some examples include TaqMan® probes, molecular beacons, SYBR GREEN® indicator dye, LUX primers, and others. The principal drawback to intercalator-based detection of PCR product accumulation, such as using SYBR GREEN® indicator dye, is that both specific and nonspecific products generate a signal. Typically, intercalators are used for single-plex detection assays and are not suitable for use for multiplex detection.

Real-time systems for quantitative PCR (qPCR) were improved by the use of probe-based, rather than intercalator-based, PCR product detection. One probe-based method for detection of amplification product without separation from the primers is the 5′ nuclease PCR assay (also referred to as the TaqMan® assay or hydrolysis probe assay). This alternative method provides a real-time method for detecting only specific amplification products. During amplification, annealing of the detector probe, sometimes referred to as a “TaqMan probe” (e.g., 5′ nuclease probe) or hydrolysis probe, to its target sequence generates a substrate that is cleaved by the 5′ nuclease activity of a DNA polymerase, such as a Thermus aquaticus (Taq) DNA polymerase, when the enzyme extends from an upstream primer into the region of the probe. This dependence on polymerization ensures that cleavage of the probe occurs only if the target sequence is being amplified.

The term “reporter,” “reporter group” or “reporter moiety” is used in a broad sense herein and refers to any identifiable tag, label, or moiety. In some embodiments, the reporter is a fluorescent reporter moiety or dye.

In general, a TaqMan detector probe can include an oligonucleotide covalently attached to a fluorescent reporter moiety or dye and a quencher moiety or dye. The reporter and quencher dyes are in close proximity, such that the quencher greatly reduces the fluorescence emitted by the reporter dye by FRET. Probe design and synthesis has been simplified by the finding that adequate quenching is typically observed for probes with the reporter at the 5′ end and the quencher at the 3′ end.

During the extension phase of PCR, if the target sequence is present, the detector probe anneals downstream from one of the primer sites and is cleaved by the 5′ nuclease activity of a DNA polymerase possessing such activity, as this primer is extended. The cleavage of the probe separates the reporter dye from quencher dye by releasing them into solution, and thereby increasing the reporter dye signal. Cleavage further removes the probe from the target strand, allowing primer extension to continue to the end of the template strand. Thus, inclusion of the probe does not inhibit the overall PCR process. Additional reporter dye molecules are cleaved from their respective probes with each cycle, affecting an increase in fluorescence intensity proportional to the amount of amplicon produced.

As used herein, the term “probe” or “detector probe” generally refers to any of a variety of signaling molecules indicative of amplification, such as an “oligonucleotide probe.” As used herein, “oligonucleotide probe” refers to an oligomer of synthetic or biologically produced nucleic acids (e.g., DNA or RNA or DNA/RNA hybrid) which, by design or selection, contain specific nucleotide sequences that allow it to hybridize under defined stringencies, specifically (i.e., preferentially) to a target nucleic acid sequence. Thus, some probes or detector probes can be sequence-based (also referred to as “sequence-specific detector probe”), for example 5′ nuclease probes. Various detector probes are known in the art, for example (TaqMan® probes described herein (See also U.S. Pat. No. 5,538,848) various stem-loop molecular beacons (See, e.g., U.S. Pat. Nos. 6,103,476 and 5,925,517 and Tyagi and Kramer, 1996, Nature Biotechnology 14:303-308), stemless or linear beacons (See, e.g., WO 99/21881), PNA Molecular Beacons™ (See, e.g., U.S. Pat. Nos. 6,355,421 and 6,593,091), linear PNA beacons (See, e.g., Kubista et al., 2001, SPIE 4264:53-58), non-FRET probes (See, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29: E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can include reporter dyes such as, for example, 6-carboxyfluorescein (6-FAM) or tetrachlorofluorescin (TET) and other dyes known to those of skill in the art. Detector probes can also include quencher moieties such as those described herein, as well as tetramethylrhodamine (TAMRA), Black Hole Quenchers (Biosearch), Iowa Black (IDT), QSY quencher (Molecular Probes), and Dabsyl and Dabcyl sulfonate/carboxylate Quenchers (Epoch). In some embodiments, detector probes can also include a combination of two probes, wherein for example a fluor is on one probe, and a quencher on the other, wherein hybridization of the two probes together on a target quenches the signal, or wherein hybridization on a target alters the signal signature via a change in fluorescence.

As used herein, a “sample” refers to any substance containing, or presumed to contain, one or more biomolecules (e.g., one or more nucleic acid and/or protein target molecules) and can include one or more of cells, a tissue or a fluid extracted and/or isolated from an individual or individuals. Samples may be derived from a mammalian or non-mammalian organism (e.g., including but not limited to a plant, virus, bacteriophage, bacteria, and/or fungus). As used herein, the sample may refer to the substance contained in an individual solution, container, vial, and/or reaction site or may refer to the substance that is partitioned between an array of solutions, containers, vials, and/or reaction sites (e.g., substance partitioned over an array of microtiter plate vials or over an array of through-holes or reaction regions of a sample plate; for example, for use in a dPCR assay). In some embodiments, a sample may be a crude sample. For example, the sample may be a crude biological sample that has not undergone any additional sample preparation or isolation. In some embodiments, the sample may be a processed sample that had undergone additional processing steps to further isolate the analyte(s) of interest and/or clean up other debris or contaminants from the sample.

As used herein, the term “amplification” or “amplify” refers to an assay in which the amount or number of one or more target biomolecules is increased, for example, by an amount to allow detection and/or quantification of the one or more target biomolecules. For example, in some embodiments, a PCR assay may be used to amplify a target biomolecule. As used herein, a “polymerase chain reaction” or a “PCR”, unless specifically defined otherwise, refers to either singleplex or multiplex PCR assays, and can be real time or quantitative PCR (wherein detection occurs during amplification) or end-point PCR (when detection occurs at the end of a PCR or after amplification; e.g., a dPCR assay). Other types of assays and methods of amplification or amplifying are also anticipated such as, for example, isothermal nucleic acid amplification and are readily understood by those of skill in the art.

As used herein, the terms “nucleic acid,” “polynucleotide,” and “oligonucleotide” can refer to primers, probes, oligomer fragments to be detected, oligomer controls —either labeled or unlabeled, and unlabeled blocking oligomers and shall be generic to polydeoxyribonucleotides (containing 2-deoxy-D-ribose), polyribonucleotides (containing D-ribose), and any other type of polynucleotide which is an N-glycoside of a purine or pyrimidine base, or modified purine or pyrimidine bases. There is no intended distinction in length between the term “nucleic acid,” “polynucleotide,” and “oligonucleotide,” and these terms will be used interchangeably. “Nucleic acid”, “DNA”, “RNA”, and similar terms can also include nucleic acid analogs. The oligonucleotides, as described herein, are not necessarily physically derived from any existing or natural sequence but may be generated in any manner, including chemical synthesis, DNA replication, reverse transcription or a combination thereof.

The term “analog” or “analogue” includes synthetic analogs having modified base moieties, modified sugar moieties, and/or modified phosphate ester moieties. As used herein, the term “modified base” refers generally to any modification of a base or the chemical linkage of a base in a nucleic acid that differs in structure from that found in a naturally occurring nucleic acid. Such modifications can include changes in the chemical structures of bases or in the chemical linkage of a base in a nucleic acid, or in the backbone structure of the nucleic acid. (See, e.g., Latorra, D. et al., Hum Mut 2003, 2:79-85. Nakiandwe, J. et al., plant Method 2007, 3:2.)

Oligonucleotides described herein, especially those functioning as a probe and/or primer, can include one or more modified bases in addition to the naturally occurring bases adenine, cytosine, guanine, thymine and uracil (represented as A, C, G, T, and U, respectively). In some embodiments, the modified base(s) may increase the difference in the Tₘ between matched and mismatched target sequences and/or decrease mismatch priming efficiency, thereby improving not only assay specificity, but also selectivity. Modified bases can be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. Such modified base(s) may include, for example, 8-Aza-7-deaza-dA (ppA), 8-Aza-7-deaza-dG (ppG), locked nucleic acid (LNA) or 2′-O,4′-C-ethylene nucleic acid (ENA) bases. Other examples of modified bases include, but are not limited to, the general class of base analogues 7-deazapurines and their derivatives and pyrazolopyrimidines and their derivatives (e.g., as described in PCT WO 90/14353, herein incorporated by reference). These base analogues, when present in an oligonucleotide, can strengthen hybridization and improve mismatch discrimination. All tautomeric forms of naturally occurring bases, modified bases and base analogues can be included. Modified internucleotide linkages can also be present in the oligonucleotides described herein. Such modified linkages include, but are not limited to, peptide, phosphate, phosphodiester, phosphotriester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art.

In some embodiments, a modified base is located at (a) the 3′-end, (b) the 5′-end, (c) at an internal position, or at any combination of (a), (b) and/or (c) in the oligonucleotide probe and/or primer.

In some embodiments the primer and/or probes as disclosed herein are designed as oligomers that are single-stranded. In some embodiments, the primers and/or probes are linear. In other embodiments, the primers and/or probes are double-stranded or include a double-stranded segment. For example, in some embodiments, the primers and/or probes may form a stem-loop structure, including a loop portion and a stem portion. In some embodiments, the primers and/or probes are short oligonucleotides, having a length of 100 nucleotides or less, more preferably 50 nucleotides or less, still more preferably 30 nucleotides or less and most preferably 20 nucleotides or less with a lower limit being approximately 3-5 nucleotides.

In some embodiments, the Tₘ of the primers and/or probes disclosed herein range from about 50° C. to about 75° C. In some embodiments, the primers and/or probes are between about 55° C. to about 65° C. In some embodiments, the primers and/or probes are between about 60° C. to 70° C. For example, the Tₘ of the primers and/or probes disclosed herein may be 56° C., 57° C., 58° C., 60° C., 61° C., 62° C., 63° C., 64° C., 65° C., 66° C., etc. In some other embodiments, the Tₘ of the primers and/or probes disclosed herein may be 56° C. to 63° C., 58° C. to 68° C. 61° C. to 69° C., 62° C. to 68° C., 63° C. to 67° C., 64° C. to 66° C., or any range in between. In some embodiments, the Tm of the primers is lower than the Tm of the probes as used herein. In some embodiments the Tm of the primers as used herein is from about 55° C. to about 65° C. and the Tm of the probes as used herein is from about 60° C. to about 70° C. In some embodiments, the Tm range of the primers used in a PCR is about 5° C. to 15° C. lower than the Tm range of the probes used in the same PCR. In yet other embodiments, the Tm of the primers and/or probes is about 3° C. to 6° C. higher than the anneal/extend temperature in the PCR cycling conditions employed during amplification.

In some embodiments, the probes include a non-extendable blocker moiety at their 3′-ends. In some embodiments, the probes can further include other moieties (including, but not limited to additional non-extendable blocker moieties that are the same or different, quencher moieties, fluorescent moieties, etc) at their 3′-end, 5′-end, and/or any internal position in between. In some embodiments, the non-extendable blocker moiety can be, but is not limited to, an amine (NH₂), biotin, PEG, DPI₃, or PO₄.

When two different, non-overlapping (or partially overlapping) oligonucleotides anneal to different regions of the same linear complementary nucleic acid sequence, and the 3′ end of one oligonucleotide points toward the 5′ end of the other, the former may be called the “upstream” oligonucleotide and the latter the “downstream” oligonucleotide.

As used herein, the terms “target sequence,” “target nucleic acid,” “target nucleic acid sequence,” and “nucleic acid of interest” are used interchangeably and refer to a desired region of a nucleic acid molecule which is to be either amplified, detected or both.

“Primer” as used herein can refer to more than one primer and refers to an oligonucleotide, whether occurring naturally or produced synthetically, which is capable of acting as a point of initiation of synthesis when placed under conditions in which synthesis of a primer extension product which is complementary to a nucleic acid strand is induced i.e., in the presence of nucleotides and an agent for polymerization such as DNA polymerase, at a suitable temperature for a sufficient amount of time and in the presence of a buffering agent. Such conditions can include, for example, the presence of at least four different deoxyribonucleoside triphosphates (such as G, C, A, and T) and a polymerization-inducing agent such as DNA polymerase or reverse transcriptase, in a suitable buffer (“buffer” includes substituents which are cofactors, or which affect pH, ionic strength, etc.), and at a suitable temperature. In some embodiments, the primer may be single-stranded for maximum efficiency in amplification. The primers herein are selected to be substantially complementary to the different strands of each specific sequence to be amplified. This means that the primers must be sufficiently complementary to hybridize with their respective strands. A non-complementary nucleotide fragment may be attached to the 5′-end of the primer (such as having a “tail”), with the remainder of the primer sequence being complementary, or partially complementary, to the target region of the target nucleic acid. Commonly, the primers are complementary, except when non-complementary nucleotides may be present at a predetermined sequence or sequence range location, such as a primer terminus as described. In some embodiments, such non-complementary “tails” can comprise a universal sequence, for example, a sequence that is common to one or more oligonucleotides. In certain embodiments, the non-complementary fragment or tail may comprise a polynucleotide sequence such as a poly (T) sequence to hybridize, for example, to a polyadenylated oligonucleotide or sequence.

The complement of a nucleic acid sequence as used herein refers to an oligonucleotide which, when aligned with the nucleic acid sequence such that the 5′ end of one sequence is paired with the 3′ end of the other, is in “antiparallel association.” Complementarity need not be perfect; stable duplexes may contain mismatched base pairs or unmatched bases.

Stability of a nucleic acid duplex is measured by the melting temperature, or “Tₘ.” The Tₘ of a particular nucleic acid duplex under specified conditions is the temperature at which half of the base pairs have disassociated.

As used herein, the term “Tₘ” or “melting temperature” of an oligonucleotide refers to the temperature (in degrees Celsius) at which 50% of the molecules in a population of a single-stranded oligonucleotide are hybridized to their complementary sequence and 50% of the molecules in the population are not-hybridized to said complementary sequence. The Tₘ of a primer or probe can be determined empirically by means of a melting curve. In some cases it can also be calculated using formulas well known in the art (See, e.g., Maniatis, T., et al., Molecular cloning: a laboratory manual/Cold Spring Harbor Laboratory, Cold Spring Harbor, N.Y.: 1982).

As used herein, the term “sensitivity” refers to the minimum amount (number of copies or mass) of a template that can be detected by a given assay. As used herein, the term “specificity” refers to the ability of an assay to distinguish between amplification from a matched template versus a mismatched template. Frequently, specificity is expressed as ΔCt=Ct_{mismatch}−Ct_{match}. In some embodiments, improvement in specificity or “specificity improvement” or “fold difference” is expressed as 2^{(ΔCt_condition1-(ΔCt_condition2)}

As used herein, the term “Ct” or “Ct value” refers to threshold cycle and signifies the cycle of a PCR amplification assay in which signal from a reporter that is indicative of amplicon generation (e.g., fluorescence) first becomes detectable above a background level. In some embodiments, the threshold cycle or “Ct” is the cycle number at which PCR amplification becomes exponential.

The term “complementary to” is used herein in relation to a nucleotide that can base pair with another specific nucleotide. Thus, for example, adenosine is complementary to uridine or thymidine and guanosine is complementary to cytidine.

The term “identical” means that two nucleic acid sequences have the same sequence or a complementary sequence.

“Amplification” as used herein denotes the use of any amplification procedures to increase the concentration of a particular nucleic acid sequence within a mixture of nucleic acid sequences.

“Polymerization”, which may also be referred to as “nucleic acid synthesis”, refers to the process of extending the nucleic acid sequence of a primer through the use of a polymerase and a template nucleic acid.

The term “label” as used herein refers to any atom or molecule which can be used to provide or aid to provide a detectable and/or quantifiable signal, and can be attached to a biomolecule, such as a nucleic acid or protein. Labels may provide signals detectable by fluorescence, radioactivity, colorimetry, gravimetry, magnetism, enzymatic activity or the like. Labels that provide signals detectable by fluorescence are also referred to herein as “fluorophores” or “fluors” or “fluorescent dyes.” As used herein, the term “dye” refers to a compound that absorbs light or radiation and may or may not emit light. A “fluorescent dye” refers to a molecule that emits the absorbed light to produce an observable detectable signal (e.g., “acceptor dyes”, “donor dyes”, “reporter dyes”, “big dyes”, “energy transfer dyes”, and the like.

In some embodiments, the term “fluorophore,” “fluor,” or “fluorescent dye” can be applied to a fluorescent dye molecule that is used in a fluorescent energy transfer pairing (e.g., with a donor dye or acceptor dye). A “fluorescent energy transfer conjugate,” as used herein typically includes two or more fluorophores (e.g., a donor dye and acceptor dye) that are covalently attached through a linker and are capable of undergoing a fluorescence energy transfer process under the appropriate conditions.

The term “quencher,” “quencher compound,” “quencher group,” “quencher moiety” or “quencher dye” is used in a broad sense herein and refers to a molecule or moiety capable of suppressing the signal from a reporter molecule, such as a fluorescent dye.

The term “overlapping” as used herein (when used in reference to oligonucleotides) refers to the positioning of two oligonucleotides on its complementary strand of the template nucleic acid. The two oligonucleotides may be overlapping any number of nucleotides of at least 1, for example by 1 to about 40 nucleotides, e.g., about 1 to 10 nucleotides or 1, 2, 3, 4, 5, 6, 7, 8, 9, or 10 nucleotides. In other words, the two template regions hybridized by oligonucleotides may have a common region which is complementary to both the oligonucleotides.

The terms “thermally cycling,” “thermal cycling,” “thermal cycles,” or “thermal cycle” refer to repeated cycles of temperature changes from a total denaturing temperature, to an annealing (or hybridizing) temperature, to an extension temperature, and back to the total denaturing temperature. The terms also refer to repeated cycles of a denaturing temperature and an extension temperature, where the annealing and extension temperatures are combined into one temperature. A total denaturing temperature unwinds all double stranded fragments into single strands. An annealing temperature allows a primer to hybridize or anneal to the complementary sequence of a separated strand of a nucleic acid template. The extension temperature allows the synthesis of a nascent DNA strand of the amplicon. The term “single round of thermal cycling” means one round of denaturing temperature, annealing temperature and extension temperature. In a single round of thermal cycling, for example, there may be internal repeating cycles of an annealing temperature and an extension temperature. For example, a single round of thermal cycling may include a denaturing temperature, an annealing temperature (i.e., first annealing temperature), an extension temperature (i.e., first extension temperature), another annealing temperature (i.e., second annealing temperature), and another extension temperature (i.e., second extension temperature).

The terms “reaction mixture,” “amplification mixture,” or “PCR mixture” as used herein refer to a mixture of components necessary to amplify at least one amplicon from nucleic acid templates. The mixture may comprise nucleotides (dNTPs), a thermostable polymerase, primers, and a plurality of nucleic acid templates. The mixture may further comprise a Tris buffer, a monovalent salt, and/or Mg²⁺. The working concentration range of each component is well known in the art and can be further optimized or formulated to include other reagents and/or components as needed by an ordinary skilled artisan.

The terms “amplified product” or “amplicon” refer to a fragment of a nucleic acid amplified by a polymerase using a pair of primers in an amplification method such as PCR or reverse transcriptase (RT)-PCR.

As defined herein, “5′→3′ nuclease activity” or “5′ to 3′ nuclease activity” or “5′ nuclease activity” refers to that activity of a cleavage reaction including either a 5′ to 3′ nuclease activity traditionally associated with some DNA polymerases, whereby nucleotides are removed from the 5′ end of an oligonucleotide in a sequential manner, (i.e., E. coli DNA polymerase I has this activity whereas the Klenow fragment does not), or a 5′ to 3′ endonuclease activity wherein cleavage occurs to more than one phosphodiester bond (nucleotide) from the -5′ end, or both, or a group of homologous 5′-3′ exonucleases (also known as 5′ nucleases) which trim the bifurcated molecules, the branched DNA structures produced during DNA replication, recombination and repair. In some embodiments, such 5′ nuclease can be used for cleavage of the labeled oligonucleotide probe annealed to target nucleic acid sequence.

As used herein, the term “phosphodiester portion” refers to a linkage comprising at least one —O—P(O)(OH)—O— functional group. It will be appreciated that a phosphodiester portion can include other groups, such as alkyl, alkylene, alkenylene, oxy-alkylene, such as PEG, in addition to one or more —O—P(O)(OH)—O— functional groups. It will be appreciated that the other groups, such as alkyl, alkylene, alkenylene, oxy-alkylene, such as PEG, can be optionally substituted with one or more substituents by replacement of one or more hydrogen atoms on the group.

As used herein, the term “protecting group” or “PG” refers to any group as commonly known to one of ordinary skill in the art that can be introduced into a molecule by chemical modification of a reactive functional group, such as an amine or hydroxyl, to obtain chemoselectivity in a subsequent chemical reaction. It will be appreciated that such protecting groups can be subsequently removed from the functional group at a later point in a synthesis to provide further opportunity for reaction at such functional groups or, in the case of a final product, to unmask such functional group. Protecting groups have been described in, for example, Wuts, P. G. M., Greene, T. W., Greene, T. W., & John Wiley & Sons. (2006). Greene's protective groups in organic synthesis. Hoboken, N.J: Wiley-Interscience. One of skill in the art will readily appreciate the chemical process conditions under which such protecting groups can be installed on a functional group. In the various embodiments described herein, it will be appreciated by a person having ordinary skill in the art that the choice of protecting groups used in the preparation of the energy transfer dye conjugates described herein can be chosen from various alternatives known in the art. It will further be appreciated that a suitable protecting group scheme can be chosen such that the protecting groups used provide an orthogonal protection strategy. As used herein, “orthogonal protection” refers to a protecting group strategy that allows for the protection and deprotection of one or more reactive functional group with a dedicated set of reaction conditions without affecting other protected reactive functional groups or reactive functional groups.

As used herein, “water-solubilizing group” refers to a moiety that increases the solubility of the compounds in aqueous solution. Exemplary water-solubilizing groups include but are not limited to hydrophilic group, as described herein, polyether, polyhydroxyl, boronate, polyethylene glycol, repeating units of ethylene oxide (—(CH₂CH₂O)—), and the like.

As used herein, “hydrophilic group” refers to a substituent that increases the solubility of the compounds in aqueous solution. Exemplary hydrophilic groups include but are not limited to —OH, —O⁻Z⁺, —SH, —S⁻Z⁺, —NH₂, —NR₃⁺Z⁻, —N═NR₂⁺Z⁻, —CN, —OCN, —SCN, —NCO, —NCS, —NO, —NO₂, —N₂⁺, —N₃, —NHC(O)R, —C(O)R, —C(O)NR₂, —S(O)₂O⁻Z⁺, —S(O)₂R, —OS(O)₂OR, —S(O)₂NR, —S(O)R, —OP(O)(OR)₂, —P(O)(OR)₂, —P(O)(O⁻)₂Z⁺, —P(O)(OH)₂, —C(O)R, —C(S)R, —C(O)OH, —C(O)OR, —CO₂⁻Z⁺, —C(S)OR, —C(S)O⁻Z⁺, —C(O)SR, —C(O)S⁻Z⁺, —C(S)SR, —C(S)S⁻Z⁺, —C(O)NR₂, —C(S)NR₂, —C(NR)NR₂, and the like, where R is H, C₁-C₆ alkyl, C₁-C₆ alkyl C₆-C₁₀ aryl, or C₆-C₁₀ aryl, and optionally substituted.

As used herein, “reactive functional group” or “reactive group” means a moiety on the compound that is capable of chemically reacting with a functional group on a different compound to form a covalent linkage, i.e., is covalently reactive under suitable reaction conditions, and generally represents a point of attachment for another substance. Typically the reactive group is an electrophile or nucleophile that can form a covalent linkage through exposure to the corresponding functional group that is a nucleophile or electrophile, respectively. In some embodiments, the “reactive functional group” or “reactive group” can be a hydrophilic group or a hydrophilic group that has been activated to be a “reactive functional group” or “reactive group.” In some embodiments, a “reactive functional group” or “reactive group” can be a hydrophilic group such as a C(O)OR group. In some embodiments, a hydrophilic group, such as a —C(O)OH, can be activated by a variety of methods known in the art to become a reactive functional group, such as by reacting the —C(O)OH group with N,N,N′,N′-tetramethyl-O—(N-succinimidyl)uronium tetrafluoroborate (TSTU) to provide the NHS ester moiety —C(O)O—NHS (a.k.a. the active ester).

Alternatively, the reactive group is a photoactivatable group that becomes chemically reactive only after illumination with light of an appropriate wavelength.

Exemplary reactive groups include, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, alkynes (including strained alkynes, such as DIBO and DBCO), azo compounds, azoxy compounds, and nitroso compounds. Reactive functional groups also include those used to prepare bioconjugates, e.g., N-hydroxysuccinimide esters (or succinimidyl esters (SE)), maleimides, sulfodichlorophenyl (SDP) esters, sulfotetrafluorophenyl (STP) esters, tetrafluorophenyl (TFP) esters, pentafluorophenyl (PFP) esters, nitrilotriacetic acids (NTA), aminodextrans, cyclooctyne-amines and the like. Methods to prepare each of these functional groups are well known in the art and their application to or modification for a particular purpose is within the ability of one of skill in the art (see, for example, Sandler and Karo, eds., Organic Functional Group Preparations, Academic Press, San Diego, 1989). Exemplary reactive groups or reactive ligands include NHS esters, phosphoramidites, and other moieties listed in Table 1 herein. Nucleotides, nucleosides, and saccharides (e.g., ribosyls and deoxyribosyls) are also considered reactive ligands due to at least their ability to form phosphodiester bonds through enzymatic catalysis. For the avoidance of doubt, saturated alkyl groups are not considered reactive ligands.

As used herein, the term “solid support,” as used herein, refers to a matrix or medium that is substantially insoluble in liquid phases and capable of binding a molecule or particle of interest. Solid supports suitable for use herein include semi-solid supports and are not limited to a specific type of support. Useful solid supports include solid and semi-solid matrixes, such as aerogels and hydrogels, resins, beads, biochips (including thin film coated biochips), microfluidic chip, a silicon chip, multi-well plate (also referred to as a microtitre plate or microplate), array (such as a microarray), membranes, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as SEPHAROSE (GE Healthcare), poly(acrylate), polystyrene, poly(acrylamide), polyol, agarose, agar, cellulose, dextran, starch, FICOLL (GE Healthcare), heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

A hydrolysis probe assay can exploit the 5′ nuclease activity of certain DNA polymerases, such as a Taq DNA polymerase, to cleave a labeled probe during PCR. One specific example of a hydrolysis probe is a TaqMan probe. In some embodiments, the hydrolysis probe contains a reporter dye at the 5′ end of the probe and a quencher dye at the 3′ end of the probe. During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the close proximity of the reporter dye to the quencher dye results in suppression of the reporter fluorescence primarily by Forster-type energy transfer (Forster, 1948; Lakowicz, 1983). During PCR, if the target of interest is present, the probe specifically anneals between the forward and reverse primer sites. The 5′ to 3′ nucleolytic activity of the Taq DNA polymerase cleaves the probe between the reporter and the quencher only if the probe hybridizes to the target. The probe fragments are then displaced from the target, and polymerization of the strand continues. In some embodiments, the 3′ end of the probe is blocked to prevent extension of the probe during PCR. In general, hybridization and cleavage process occurs in sequential cycles and does not interfere with the exponential accumulation of the product.

Without being bound to these parameters, the general guideline for designing TaqMan probes and primers is as follows: design the primers as close as possible to, but without overlapping the probe; the Tₘ of the probe should be about 10° C. higher than the Tₘ of the primers; select the strand that gives the probe more C than G bases; the five nucleotides at the 3′ end of the primer should have no more than two G and/or C bases, and the reaction should be run on the two-step thermal profile with the annealing and extension under the same temperature of 60° C.

The present disclosure further relates to kits for detecting at least one biomolecule in a sample, the kit comprising a compound of Formula (I) or Formula (II) and at least one excipient, and instructions for use of the compound to detect a biomolecule in a sample.

In some embodiments, kits are provided comprising a compound disclosed herein and/or a conjugate disclosed herein and one or more other reagents. Any embodiment of a compound described herein can be provided in a kit. In some embodiments, at least one additional reagent described above is included in a kit with a compound according to this disclosure. In certain embodiments, the kits further comprise one or more of the following: a buffering agent, a purification medium, or a vial comprising a sample. In some embodiments, a kit further comprises a cytotoxic agent, apoptosis inducer, cells, a solvent, or a desiccant. Kits may also include reagents and instructions useful to conduct the methods of the disclosure, e.g., conjugation methods, assay methods, or synthetic methods. In some embodiments, kits further comprise at least one additional substance such as a solvent, buffer, stabilizer, pH adjusting agent, etc. In some embodiments, the kit further comprises an antifade reagent.

The following description of cyanine dye compounds provides general information regarding construction of the compounds and probes described herein. As described herein, the cyanine compounds can be covalently bound, optionally through a linker, to form an energy transfer dye pair with a quencher moiety. In some embodiments, a reporter moiety and a quencher moiety can be covalently bound to one another through an analyte. In some embodiments the analyte is a probe, such as an oligonucleotide probe.

The following abbreviations may be relevant for the application.

Abbreviations

| | |
|---|---|
| (D)PBS | (Dulbecco's) phosphate buffered saline |
| (o-Ts)₃P | tris(o-tolyl)phospine |
| abs | absorbance |
| Ac₂O | acetic anhydride |
| ACN | acetonitrile |
| AcOEt | ethylacetate |
| AcOH | acetic acid |
| AcONa | sodium acetate |
| AM | aminomethyl |
| aq | aqueous |
| Ar | argon |
| BINAP | (2,2′-bis(diphenylphosphino)-1,1′-binaphthyl |
| cat | catalytic |
| COMU | (1-cyano-2-ethoxy-2-oxoethylidenaminooxy) |
| | dimethylamino-morpholino-carbenium |
| | hexafluorophosphate |
| conc | concentrated |
| D | dimension |
| DBU | 1,8-diazabicyclo[5.4.0]undec-7-ene |
| DCM | dichloromethane |
| DIEA | diethylamine |
| DIPEA | diisopropylethylamine |
| DMA | dimethylacetamide |
| DMF | dimethylformamide |
| DMF.DMA | dimethylformamide/dimethylacetamide |
| DMSO | dimethylsulfoxide |
| DMTr | dimethoxytrityl |
| DSC | differential scanning calorimetry |
| EDC | triethylammonium acetate |
| em | emission |
| eq, equiv | Equivalence |
| Et₂O | diethyl ether |
| Et₃N | triethylamine |
| EtOAc | ethyl acetate |
| EtOH | ethanol |
| h | hour(s) |
| HMBC | heteronuclear multiple bond correlation |
| HOAc | acetic acid |
| HSQC | heteronuclear single quantum coherence |
| IEX | ion-exchange |
| IPA | isopropyl alcohol |
| iPrOAc | isopropylacetate |
| L | liter |
| LCMS | liquid chromatography mass spectrometry |
| M | molar concentration |
| MeCN | acetonitrile |
| MeI | iodomethane |
| MeNPh | N-methylaniline |
| MeOH | methanol |
| MeSO₃H | methanesulfonic acid |
| mg | milligram |
| min | minute(s) |
| mL | milliliter |
| MsOH | methanesulfonic acid |
| MTBE | methyl tert-butyl ether |
| N | normal concentration |
| NaOtBu | sodium tert-butoxide |
| NHS | N-hydroxysuccinimide |
| nm | nanometer |
| NMR | nuclear magnetic resonance |
| ODMT | O-dimethoxytrityl |
| ODU | optical density units |
| PCR | polymerase chain reaction |
| Pd(OAc)₂ | palladium (2) acetate |
| PMT | photomultiplier tube |
| PPA | polyphosphoric acid |
| p-TsOH | p-toluenesulfonic acid |
| Q₃PA | quinone propanoic acid |
| QY | quantum yield |
| RP | reversed phase |
| rt | room temperature |
| sat | saturated |
| T3P | propylphosphonic anhydride |
| TEA | triethylamine |
| TEAA | triethylammonium acetate |
| TFA | trifluoroacetic acid |
| TG | thermogravimetry |
| THF | tetrahydrofuran |
| TLC | thin layer chromatography |
| TSTU | N,N,N′,N′-tetramethyl-O-(N-succinimidyl)uronium |
| | tetrafluoroborate |
| U(H)PLC | ultra (high) performance liquid chromatography |
| vol. | volume |

The following non-limiting examples further describe various ones of the compounds, methods, compositions, uses, and embodiments disclosed herein.

### EXAMPLES

### Example 1: Synthesis of Compound 18

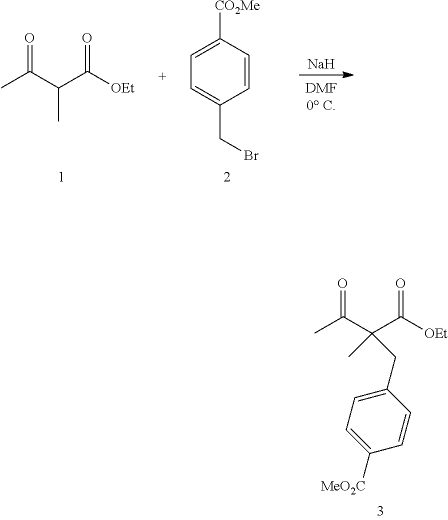

Step 1: Synthesis of methyl 4-(2-(ethoxycarbonyl)-2-methyl-3-oxobutyl)benzoate (3).

Ethyl 2-methylacetoacetate (3.46 g, 24 mmol) dissolved in 3 mL of DMF was added slowly dropwise to a suspension of NaH (1.1 g, 24 mmol, 60% in paraffin) in 20 mL of DMF at 0° C. After warming to room temperature for 30 min. a cloudy white suspension resulted. This was cooled to 0° C. and methyl 4-(bromomethyl)benzoate in 8 mL DMF was added dropwise. The resulting clear solution was warmed to room temperature and then stirred for 1.5 h then 60° C. for 20 min. The reaction solution was diluted with DCM and washed with 5% ammonium chloride, water, and then brine. The DCM layer was dried over anhydrous sodium sulfate, filtered, and concentrated to a clear, colorless oil. This was purified by column chromatography on silica gel eluting with 20% EtOAc/Hexane to yield 5.94 g of a colorless oil (3).

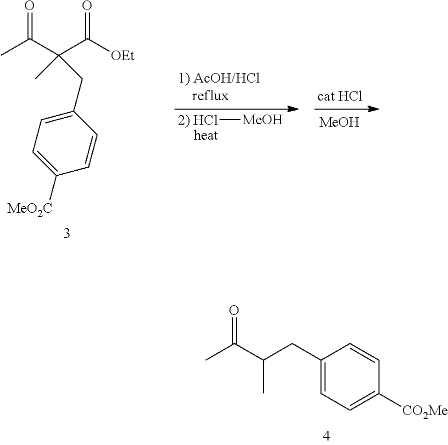

### Step 2: Synthesis of methyl 4-(2-methyl-3-oxobutyl)benzoate (4)

Compound 3 was decarboxylated and de-esterified by diluting in 20 mL AcOH and 7 mL cone HCl and refluxed for 48 h. The solution was concentrated by co-evaporating with toluene and DCM to a white solid. The product was re-esterified by adding 40 mL of 1 M HCl/MeOH and heating at 60° C. for 2.5 h. The solution was concentrated, co-evaporated with DCM several times to yield 4.06 g (85%) of a clear pale orange-colored oil (4). LCMS calculated 221.12, observed 221.12.

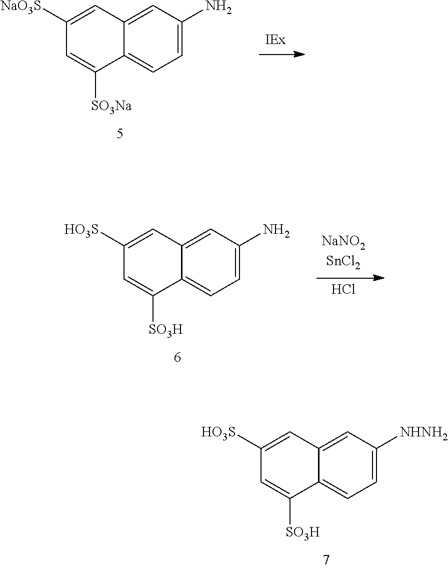

### Step 3: Synthesis of 6-hydrazineylnaphthalene-1,3-disulfonic acid (7)

40 mL of Dowex IEX resin was washed with 120 mL 0.1 M sulfuric acid for 30 min. The resin was filtered and washed with water until a neutral pH was obtained. 6-Amino-1,3-naphthalenedisulfonic acid disodium salt (5, 5.00 g, 14.4 mmol) was mixed with 40 mL of water and the IEX resin for 2 h. The mixture was filtered, and the filtrate concentrated and dried under vacuum. 6 was then mixed with 100 mL 1:1 conc HCl/water and cooled to 0° C. To this was added sodium nitrite (1.49 g, 21.6 mmol) in 100 mL of water dropwise. After stirring for 15 min at 0° C., a solution of tin (II) chloride (13.6 g. 60.5 mmol) in 10 mL of conc HCl was added to the cold thick mixture. After addition, the clear solution was stirred for 2 h at 0° C. A yellow precipitate was collected and washed with minimal 2-propanol. A second crop of solid was obtained from the concentrated filtrate in minimal 2-propanol to yield 4.47 g (98% yield) of 7 as a pale-yellow solid. This was used without further purification.

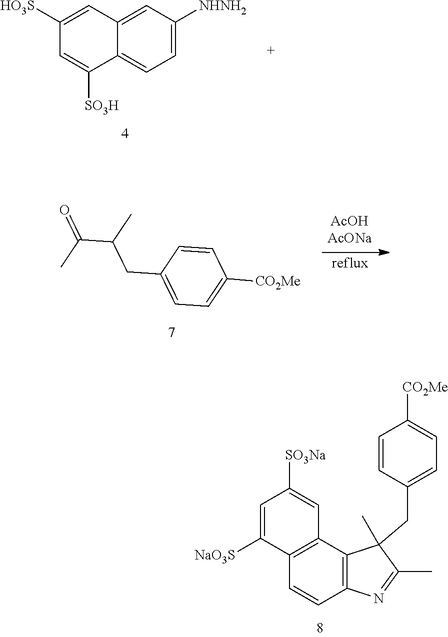

### Step 4: Synthesis of sodium 1-(4-(methoxycarbonyl)benzyl)-1,2-dimethyl-1H-benzo[e]indole-6,8-disulfonate (8)

Compound 4 (2.94 g, 9.22 mmol), 7 (2.64 g, 220.27 mmol), sodium acetate (2.65 g, 32.3 mmol), and 30 mL acetic acid were mixed and heated at reflux for 3 h. The mixture was concentrated and co-evaporated with DCM/hexane to a brown solid. This was purified by column chromatography on silica gel eluting with DCM:MeOH:water:acetic acid (6.5:3:0.2:0.1) to yield 4.37 g (87% yield) of 8 as a yellow solid.

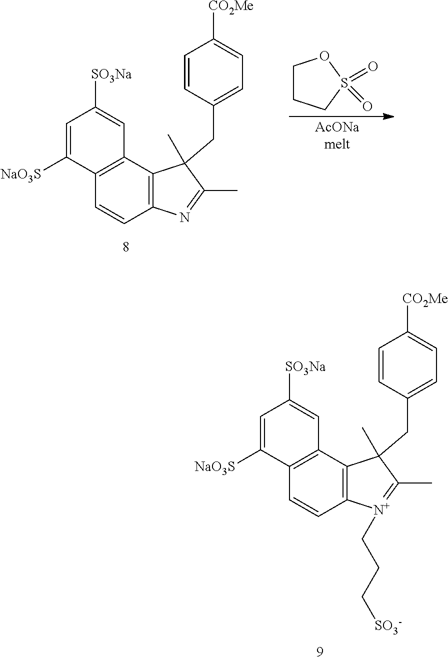

### Step 5: Synthesis of sodium 1-(4-(methoxycarbonyl)benzyl)-1,2-dimethyl-3-(3-sulfonatopropyl)-1H-benzo[e]indol-3-ium-6,8-disulfonate (9)

Compound 8 (2.00 g, 3065 mmol), sodium acetate (0.90 g, 11.0 mmol), and propane sultone (2.67 g, 21.9 mmol) were mixed well in acetonitrile and then concentrated to make a homogeneous mixture. This was briefly heated at 100° C. (12 min). Upon cooling, 20 mL of MeOH was added followed by excess (200 mL) EtOAc to obtain a tan precipitate which was washed with EtOAc. This was purified by column chromatography on RP media eluting with water to yield 2.14 g (88% yield) of 9 as an orange-brown solid. LCMS calculated 626.08, observed 626.08.

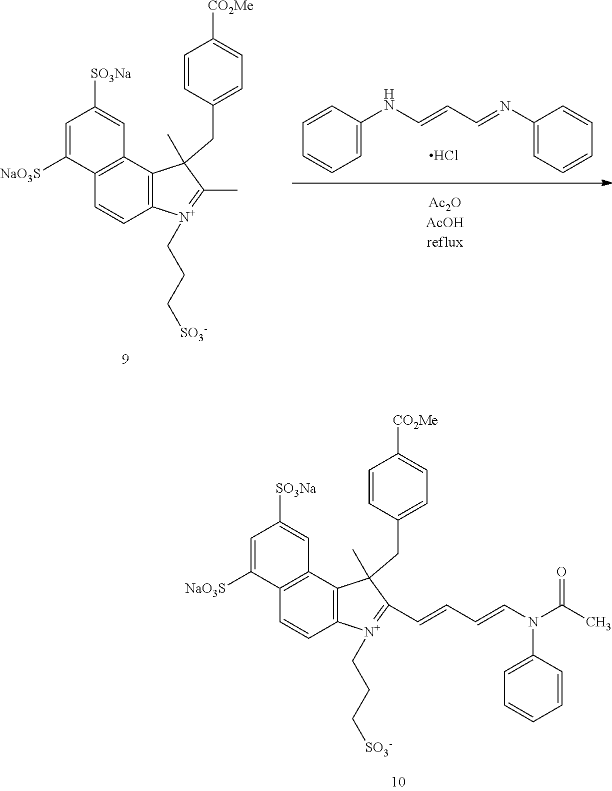

### Step 6: Synthesis of Compound 10

Compound 9 (20 mg, 0.030 mmol), malonaldehyde dianilide HCl (7.7 mg, 0.030 mmol), 500 uL of acetic anhydride, and 500 uL of acetic acid were heated at 120° C. for 2 h. The reaction solution was mixed with excess EtOAc to obtain a brown-reddish precipitate. This was washed with EtOAc, Et₂O, and then dried to yield 13 mg (52%) of 10 as an orange-brown powder. This was used without further purification. LCMS calculated 797.15, observed 797.15.

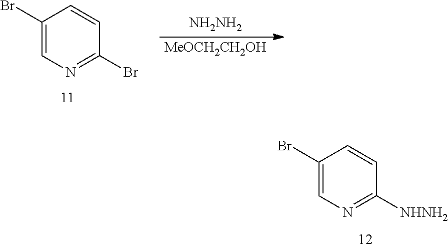 [SMILES: "MeOCH2CH2OH" ; Brc1ccc(Br)nc1 ; "NH2NH2" ; NNc1ccc(Br)cn1]

### Step 7: Synthesis of 5-bromo-2-hydrazineylpyridine (12)

2-5-Dibromopyridine (11, 12.00 g, 50.9 mmol) and anhydrous hydrazine (15.8 mL, 50.9 mmol) were mixed in 50 mL of 2-methoxyethanol and heated at 110° C. for 2 h. After concentration, the residue was mixed with DCM and washed with water and brine, and then dried over anhydrous sodium sulfate, filtered, and concentrated. Further drying under vacuum yielded 7.99 g (84% yield) of 12 as an off-white solid. LCMS calculated 187.98, observed 187.98.

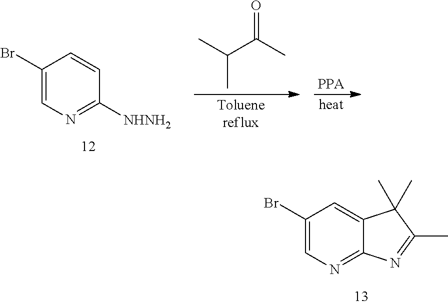

### Step 8: Synthesis of 5-bromo-2,3,3-trimethyl-3H-pyrrolo[2,3-b]pyridine (13)

5-Bromo-2-hydrazineylpyridine (7.99 g, 42.5 mmol), 3-methylbutan-2-one (7.33 g, 85.0 mmol), and 50 mL of toluene were refluxed using a Dean-Stark trap for 3 h. The reaction solution was concentrated and then mixed with 30 g of polyphosphoric acid, and the mixture was heated at 145° C. for 3 h. The hot reaction resin was poured into ice water and neutralized with NaOH pellets until a pH of 7-8. The mixture was extracted 3 times into EtOAc, washed with brine, dried over anhydrous sodium sulfate, filtered, and concentrated. This was purified by column chromatography on silica gel eluting with 50% EtOAc/hexane to yield 2.14 g (21% yield) of 13 as an orange-colored solid. LCMS calculated 239.02 observed 239.02.

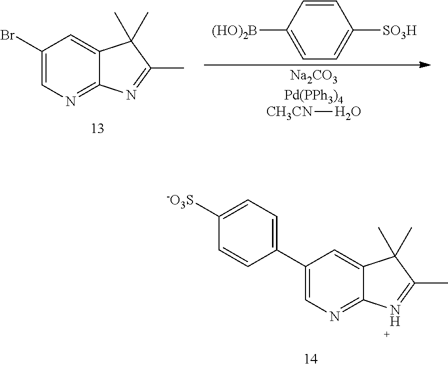

### Step 9: Synthesis of 4-(2,3,3-trimethyl-3H-pyrrolo[2,3-b]pyridin-5-yl)benzenesulfonic acid (14)

5-bromo-2,3,3-trimethyl-3H-pyrrolo[2,3-b]pyridine (13, 1.97 g, 8.25 mmol), 4-boronobenzenesulfonic acid (2.00 g, 9.90 mmol), sodium carbonate (3.32 g, 31.4 mmol), and tetrakis(triphenylphosphine) palladium (0) (1.91 g, 1.65 mmol) were mixed in 150 mL of 1:1 acetonitrile-water. The mixture was degassed with N₂ for 10 min and refluxed for 21 h. Upon cooling the mixture was concentrated and purified by column chromatography on silica gel eluting with 20 to 30% MeOH/DCM to yield 2.41 g (92% yield) of 14 as a pale-yellow solid. ¹H NMR (500 MHz, DMSO-d₆): δ 8.62 (s, 1H), 8.22 (s, 1H), 7.72 (s, 4H), 2.30 (s, 3H), 1.24 (s, 6H); ¹³C NMR (500 MHz, DMSO-d₆) δ 193.52, 166.65, 147.48, 145.69, 138.76, 137.56, 131.92, 128.47, 126.26, 52.41, 21.95, 15.44; LCMS calculated 317.10 observed 317.10. 3106-160, 3109-107; 3142-124.

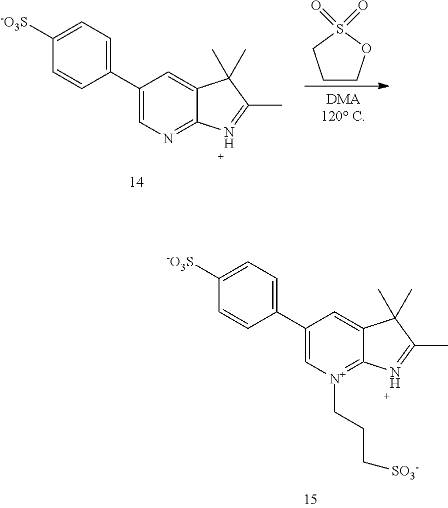

### Step 10: Synthesis of 3-(2,3,3-trimethyl-5-(4-sulfophenyl)-3H-pyrrolo[2,3-b]pyridin-7-ium-7-yl)propane-1-sulfonate (15)

Compound 14 (0.66 g, 2.09 mmol) and propane sultone (1.10 mL, 12.5 mmol) were combined with 20 mL dimethylacetamide and heated at 120° C. for 1 h. The reaction solution was mixed with 175 mL EtOAc and the resulting precipitate was washed with EtOAc and dried. The pale purple solid (15) was used without further purification. LCMS calculated 439.099, observed 439.093.

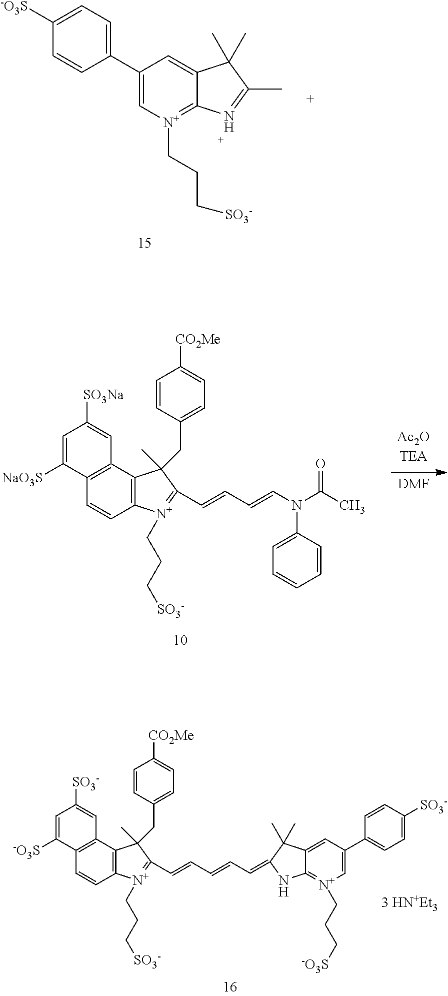

### Step 7: Synthesis of Compound 16

Compound 10 (13 mg, 0.0118 mmol) and compound 15 (8.1 mg, 0.018 mmol) were dissolved in 2 mL DMF. Acetic anhydride (32 uL) and TEA (65 uL) were added, and the amber solution was stirred at room temperature for 3 h. The reaction solution was partially concentrated to reduce the DMF volume and then precipitated with EtOAc. The solid was washed with EtOAc and Et₂O, and then dried. This was purified by column chromatography on RP media eluting with 0 to 10% MeOH/water to yield 6.4 mg (30% yield) of 16 as a dark blue solid. LCMS calculated 1099.17, observed 1099.16.

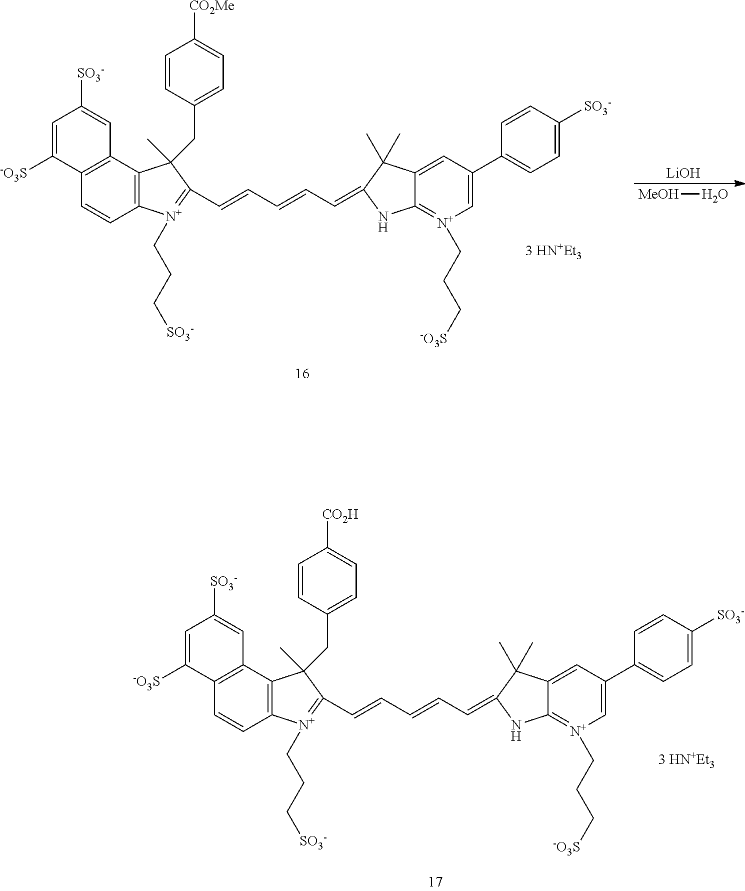

### Step 8: Synthesis of Compound 17

Compound 16 (6.4 mg, 0.0055 mmol) was dissolved in 1 mL MeOH and mixed with 1 mL of 1 M aq LiOH. After stirring for 4 h at room temperature, 200 uL of AcOH was added to neutralize to pH 7. The solution was concentrated, and the residue desalted on a small pad of C₁₈ media, washing with water and eluting with MeOH. Upon concentration and drying, 4 mg (67% yield) of 17 was obtained as a dark blue solid. LCMS calculated 1085.15, observed 1086.14.

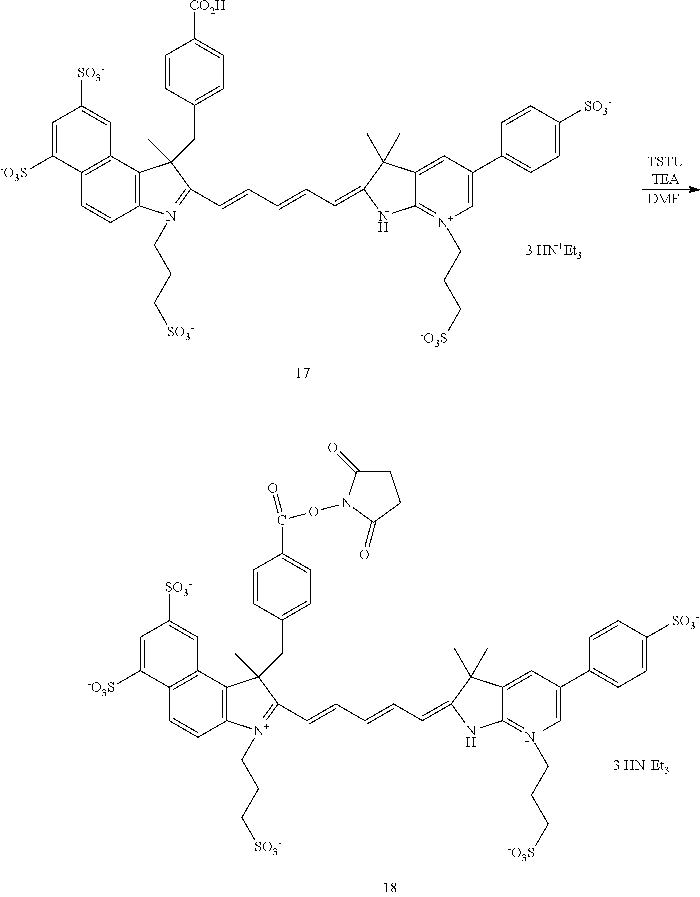

### Step 9: Synthesis of Compound 18

Compound 17 (4.0 mg, 0.0035 mmol) was dissolved in 500 μL of anhydrous DMF.

TEA (1.5 μL, 0.011 mmol) and N,N,N,N-tetramethyl-O—(N-succinimidyl)uranium tetrafluoroborate (TSTU, 1.3 mg, 0.0042 mmol) were added and the solution was stirred at room temperature for 4.5 h. The product was precipitated by adding excess EtOAc. The solid was washed with EtOAc and Et₂O, and then dried under vacuum to yield 3.0 mg (68% yield) of 18 as a blue solid. LCMS calculated 1182.17, observed 1182.16.

### Example 2: Synthesis of Compound 30

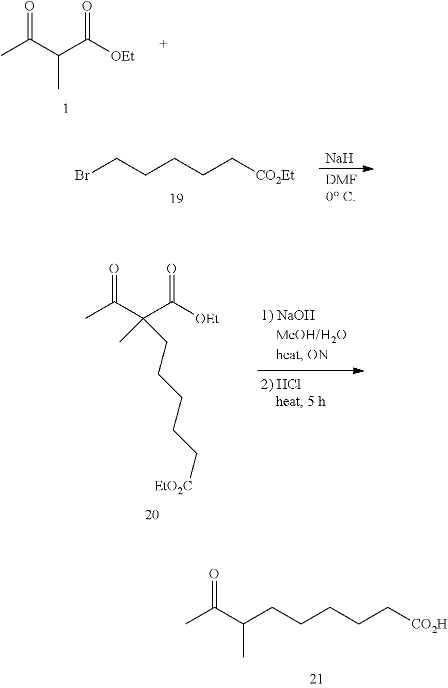

### Step 1: Synthesis of 7-methyl-8-oxononanoic acid (21)

Ethyl 2-methylacetoacetate (1, 61.14 g, 424 mmol) was dissolved in 30 mL DMF. This was added dropwise to a suspension of NaH (17.0 g, 60% in paraffin, 424 mmol) in 120 mL DMF at 0° C. After warming to room temperature for 30 min, the reaction mixture was cooled to 0° C., and ethyl 6-bromohexanoate (19, 75.4 mL, 424 mmol) dissolved in 30 mL DMF was added dropwise. The cloudy reaction mixture was warmed to 60° C. and stirred for 3.5 h. After cooling, the mixture was diluted with EtOAc and washed with 5% ammonium chloride and water, and then dried over anhydrous sodium sulfate, filtered, and concentrated to a yellow oil. The oil, 20, was diluted in 1 L MeOH and heated with 17% aq NaOH at 60° C. for 1 h. MeOH was removed by rotary evaporation and the aqueous residue was cooled to 0° C. and 100 mL of conc HCl was added slowly dropwise. The resulting mixture was extracted twice with EtOAc and the organic layer washed with brine and concentrated. This was purified by column chromatography on silica gel eluting with 30 to 60% EtOAc/hexane to yield 72.47 g (92%) of 21 as a pale-yellow oil. ¹H NMR (500 MHz, CDCl₃) δ 2.4 (q, 1H), 2.3 (t. 3H), 2.1 (s, 3H), 1.6 (m, 4H), 1.4-1.2 (m, 6H), 1.1 (d, 3H).

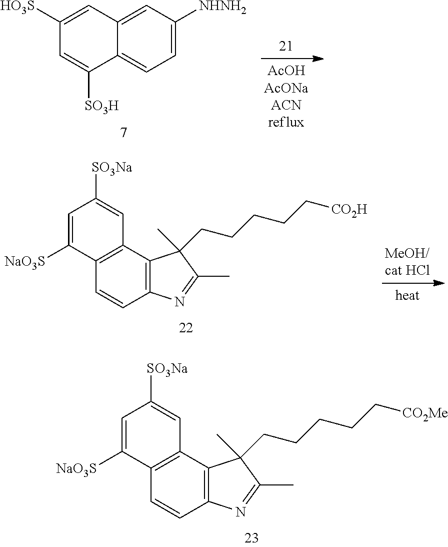

### Step 2: Synthesis of Compound 23

6-Hydrazineylnaphthalene-1,3-disulfonic acid (7, 2.92 g, 9.19 mmol), 7-methyl-8-oxononanoic acid (21, 2.22 g, 11.9 mmol), sodium acetate (2.63 g, 32.1 mmol), and 35 mL of acetic acid were refluxed together for 2 h. The reaction mixture was concentrated, and the residue stirred with 50 mL 0.5 M HCl/MeOH at 60° C. for 2 h. The suspension was concentrated and purified by column chromatography using RP media eluting with water to yield 1.34 g (28% yield) of 23 as an amber foamed solid.

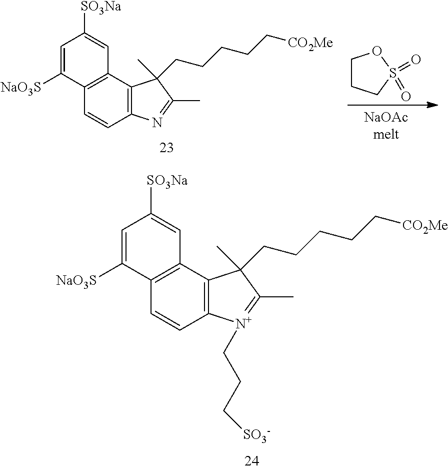

### Step 3: Synthesis of Compound 24

Compound 23 (1.35 g, 2.65 mmol), sodium acetate (0.63 g, 7.68 mmol), and propane sultone (1.35 mL, 15.4 mmol) were mixed well together and heated at 110° C. for 1 h. The cooled resin was dissolved in 15 mL of MeOH and precipitated from 150 mL EtOAc. The tan solid was washed with EtOAc, dried and purified by column chromatography on RP media eluting with water to yield 1.29 g (78% yield) of 24 as a pale amber solid. LCMS calculated 606.11, observed 606.11.

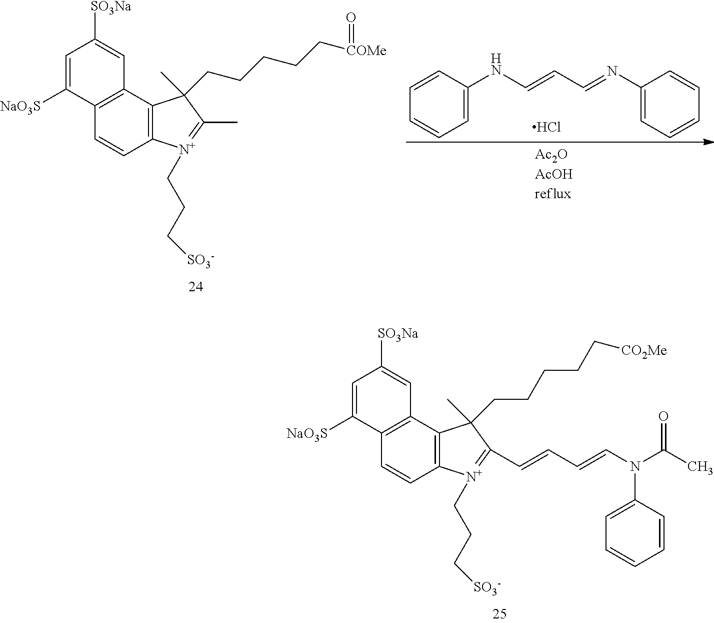

### Step 4: Synthesis of Compound 25

Compound 24 (10 mg, 0.015 mmol), malonaldehyde dianalide HCl (7.8 mg, 0.030 mmol), 500 uL acetic anhydride, and 500 μL acetic acid were mixed well and heated at 120° C. for 2 h. Product was precipitated by adding excess EtOAc. This was washed with EtOAc and dried to yield 11 mg (92% yield) of 25 as an amber-red solid. This was used without further purification. LCMS calculated 777.18, observed 777.18.

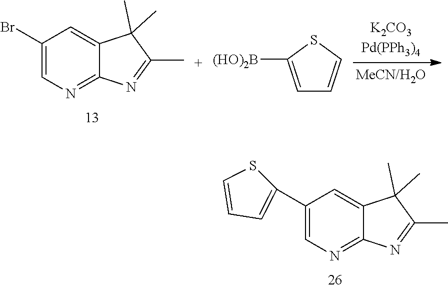 [SMILES: CC1=Nc2ncc(Br)cc2C1(C)C ; OB(O)c1cccs1 ; CC1=Nc2ncc(-c3cccs3)cc2C1(C)C]

### Step 5: Synthesis of 2,3,3-trimethyl-5-(thiophen-2-yl)-3H-pyrrolo[2,3-b]pyridine, 26

5-bromo-2,3,3-trimethyl-3H-pyrrolo[2,3-b]pyridine (13, 100 mg, 0.42 mmol), thiophene-2-boronic acid (75 mg, 0.59 mmol), sodium carbonate (16.9 mg, 1.6 mmol) and tetra-kis(triphenylphosphine) palladium (0) (47 mg, 0.084 mmol) were mixed in 10 mL acetonitrile-water (1:1) and degassed with N₂ for 5 min. The mixture was refluxed 8 h. The mixture was dispersed in water, extracted into EtOAc three times, dried over anhydrous sodium sulfate, filtered, and concentrated to an amber-yellow oil. This was purified by column chromatography on silica gel eluting with 10% MeOH/EtOAc to give 60 mg (59% yield) of 26 as an amber solid. LCMS calculated 243.10, observed 243.09.

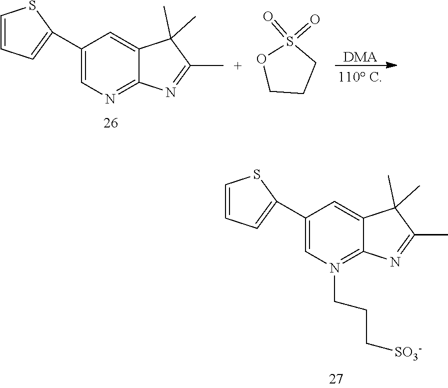

### Step 6: Synthesis of Compound 27

Compound 26 (60 mg, 0.25 mmol) and propane sultone (88 uL, 1.0 mmol) were dissolved in 2 mL of dimethylacetamide and heated at 110° C. for 1 h. Upon cooling, excess EtOAc was added and the product precipitated. The solid was washed with EtOAc and Et₂O, and dried to yield 50 mg (55% yield) of 27 as a grey solid. The product was used without further purification. LCMS calculated 365.10, observed 365.10.

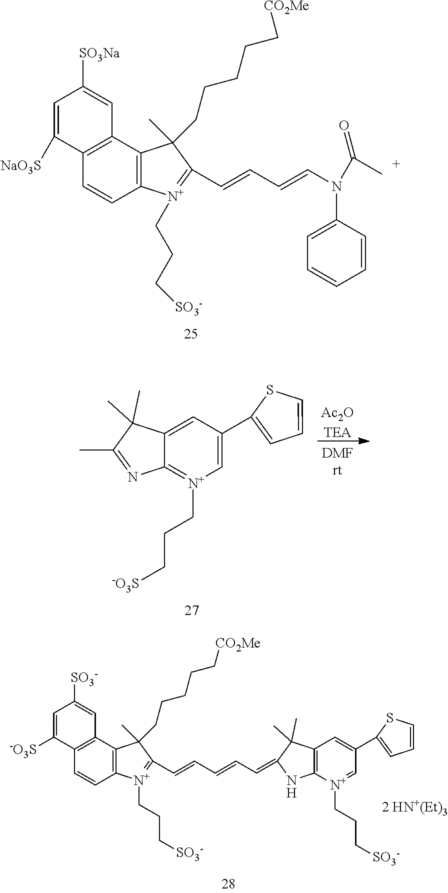

### Step 7: Synthesis of Compound 28

Compound 25 (11 mg, 0.013 mmol), compound 27 (4.9 mg, 0.013 mmol), acetic anhydride (4.9 uL, 0.052 mmol), and TEA (11 ul, 0.078 mmol) were mixed in 2 mL of DMF at room temperature for 5 h. The reaction solution was concentrated to a reduced volume and then precipitated with 45 mL EtOAc. The precipitate was washed with EtOAc and then dried. This was purified by column purification using RP media eluting with 5 to 50% MeOH/water to yield 4.6 mg (27% yield) of 28 as a dark blue solid. LCMS (negative) calculated 1006.20, observed 1006.24.

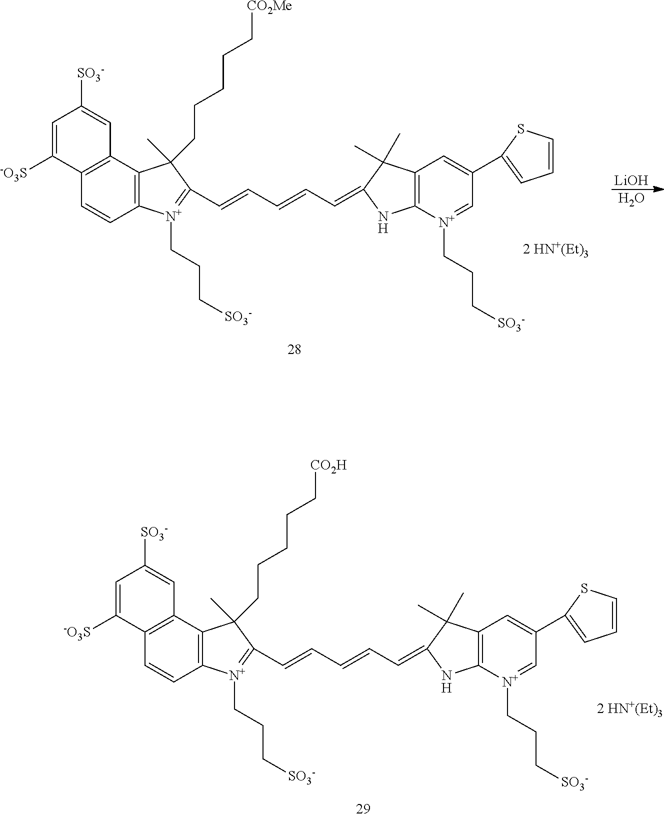

### Step 8: Synthesis of Compound 29

Compound 28 (4.6 mg, 0.0035 mmol) was dissolved in 1 mL MeOH and mixed with 1 mL of 1 M aq LiOH. After stirring for 1 h at room temperature, 200 uL of AcOH was added to neutralize to pH 7. The solution was concentrated, and the residue desalted on a small pad of C₁₈ media, washing with water and eluting with MeOH. Upon concentration and drying, 4 mg (100% yield) of 29 was obtained as a dark blue solid. LCMS calculated (negative) 991.18, observed 991.42. Excitation/Emission 705 nm/733 nm.

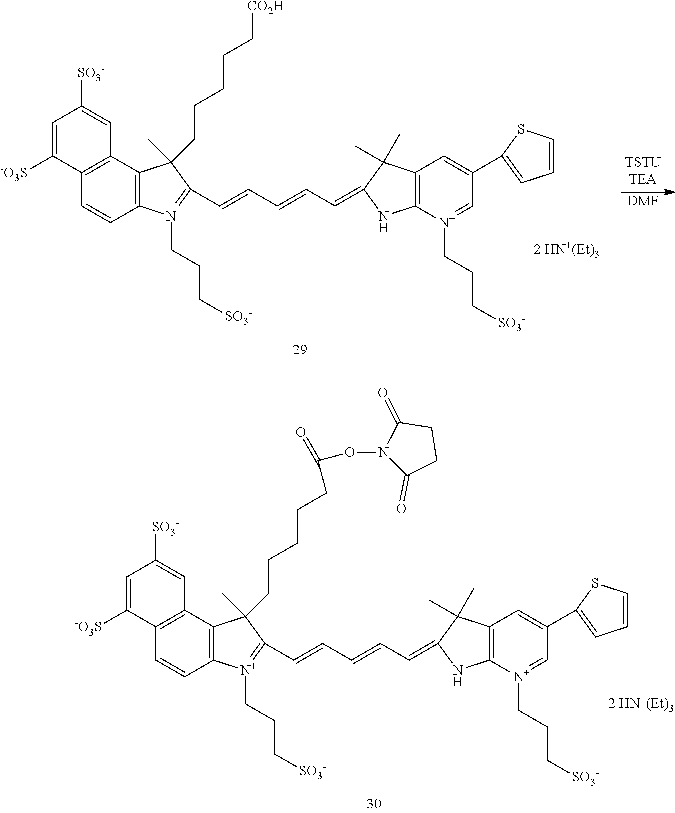

### Step 9: Synthesis of Compound 30

Compound 29 (4.0 mg, 0.0040 mmol) was dissolved in 500 μL of anhydrous DMF.

TEA (1.7 μL, 0.012 mmol), and N,N,N,N-tetramethyl-O—(N-succinimidyl)uranium tetrafluoroborate (TSTU, 1.4 mg, 0.0048 mmol) were added and the solution stirred at room temperature for 1.5 h. The product was precipitated by adding excess EtOAc. The solid was washed with EtOAc and dried under vacuum to yield 5.0 mg (89% yield) of 30 as a blue solid. LCMS (negative) calculated 1089.20, observed 1089.21.

### Example 3: Synthesis of Compound 36

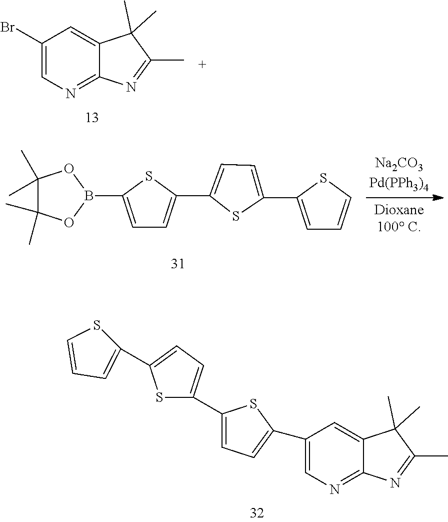 [SMILES: CC1=Nc2ncc(Br)cc2C1(C)C ; CC1(C)OB(c2ccc(-c3ccc(-c4cccs4)s3)s2)OC1(C)C ; CC1=Nc2ncc(-c3ccc(-c4ccc(-c5cccs5)s4)s3)cc2C1(C)C]

### Step 1: Synthesis of Compound 32

Compound 13, (100 mg, 0.418 mmol), 2,2′,5′2″-terthhiophene-5-boronic acid pinacol ester (235 mg, 0.627 mmol), sodium carbonate (177 mg, 0.0836 mmol), and tetrakis(triphenylphosphine) palladium (0) (31, 97 mg, 0.0836 mmol) were mixed in 10 mL of acetonitrile-water (1:1) and degassed with N₂ for 5 min. The mixture was heated at 100° C. for 4 h, and then the cooled reaction mixture was dispersed in water and extracted three times with EtOAc, dried over anhydrous sodium sulfate, filtered, and concentrated. This was purified by column chromatography on silica gel eluting with 5 to 10% MeOH/EtOAc to yield 45 mg (26% yield) of 32 as an amber-yellow solid. LCMS calculated 407.07, observed 407.07.

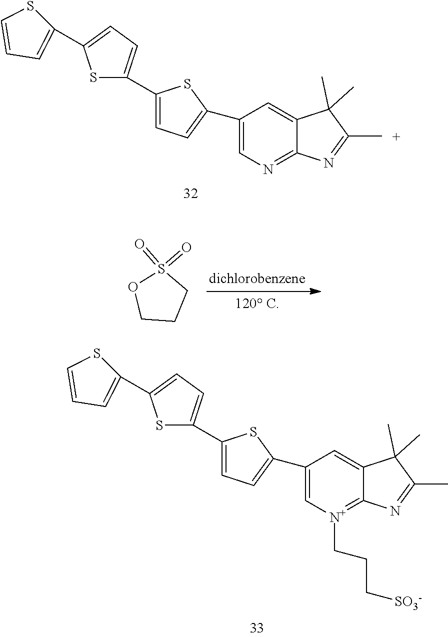

### Step 2: Synthesis of Compound 33

Compound 32 (45 mg, 0.11 mmol) and propane sultone (39 uL, 0.44 mmol) were dissolved in 2 mL dichlorobenzene and heated at 120° C. for 1 h. The reaction solution was mixed with 45 mL EtOAc, and the precipitated product was washed with EtOAc, concentrated, and dried to yield 37 mg (64% yield) of 33 as a yellowish solid. LCMS calculated 529.07, observed 529.07.

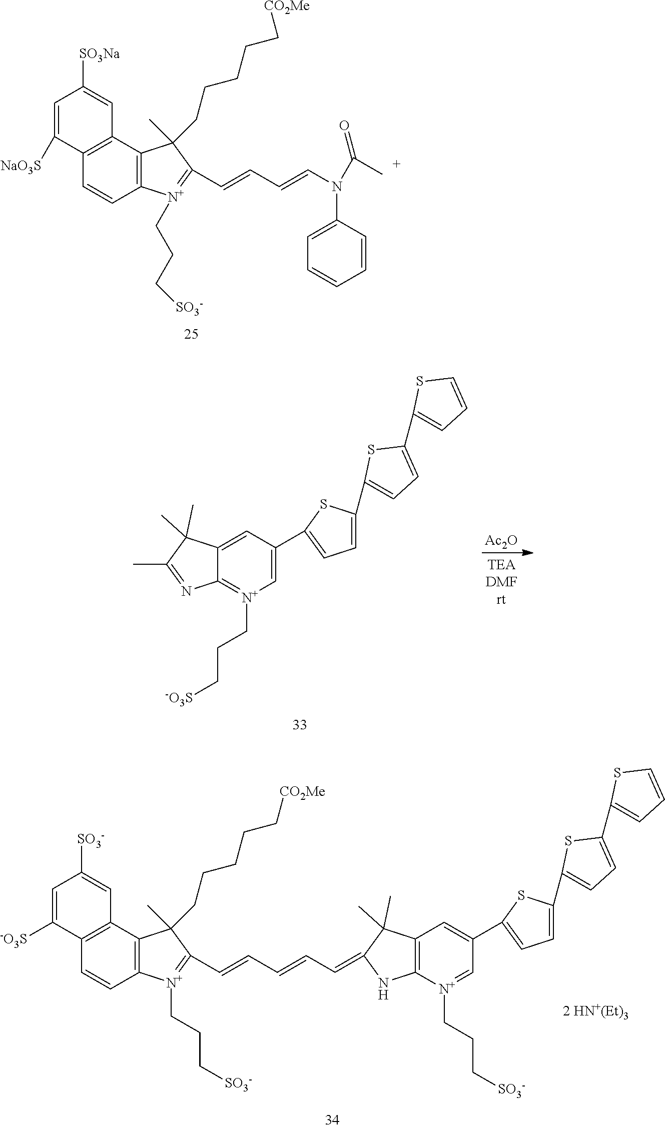

### Step 3: Synthesis of Compound 34

Compound 25 (32 mg, 0.039 mmol), compound 33 (24 mg, 0.045 mmol), acetic anhydride (55 uL, 0.59 mmol), and TEA (163 ul, 1.17 mmol) were mixed in 4 mL of DMF at room temperature for 5 h. The reaction solution was mixed with 45 mL EtOAc, and the precipitated solid was washed with EtOAc and then dried. This was purified by column purification using RP media eluting with 0 to 50% MeOH/water to yield 10 mg (18% yield) of 34 as a dark blue solid. LCMS (negative) calculated 1170.18, observed 1170.20.

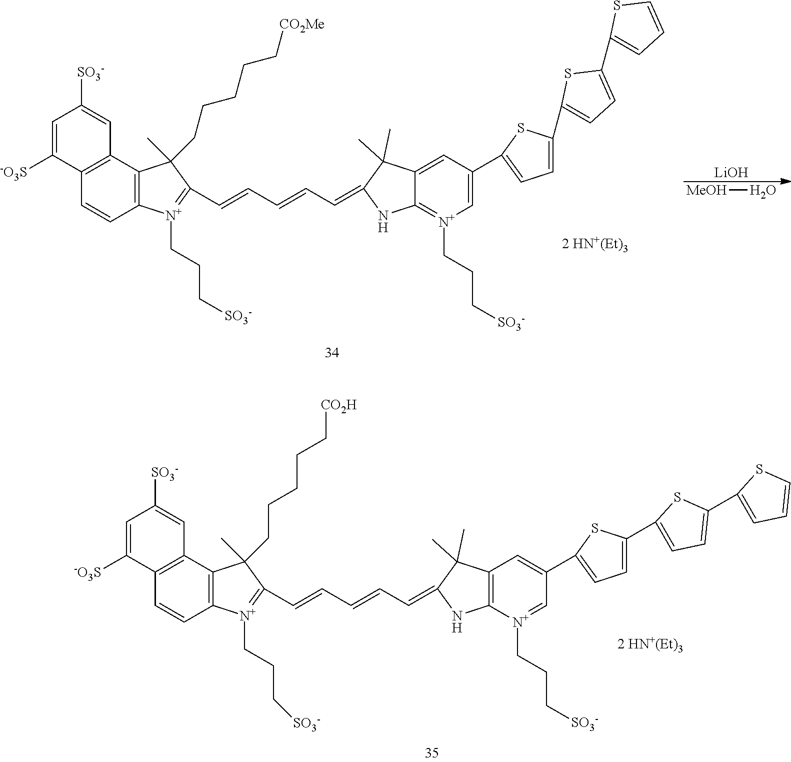

### Step 4: Synthesis of Compound 35

Compound 35 was synthesized in a manner similar to compound 29 to yield 11 mg of 35 as dark blue solid. LCMS (negative) 1156.16, observed 1156.16. Excitation/emission in TE buffer 718 nm/741 nm.

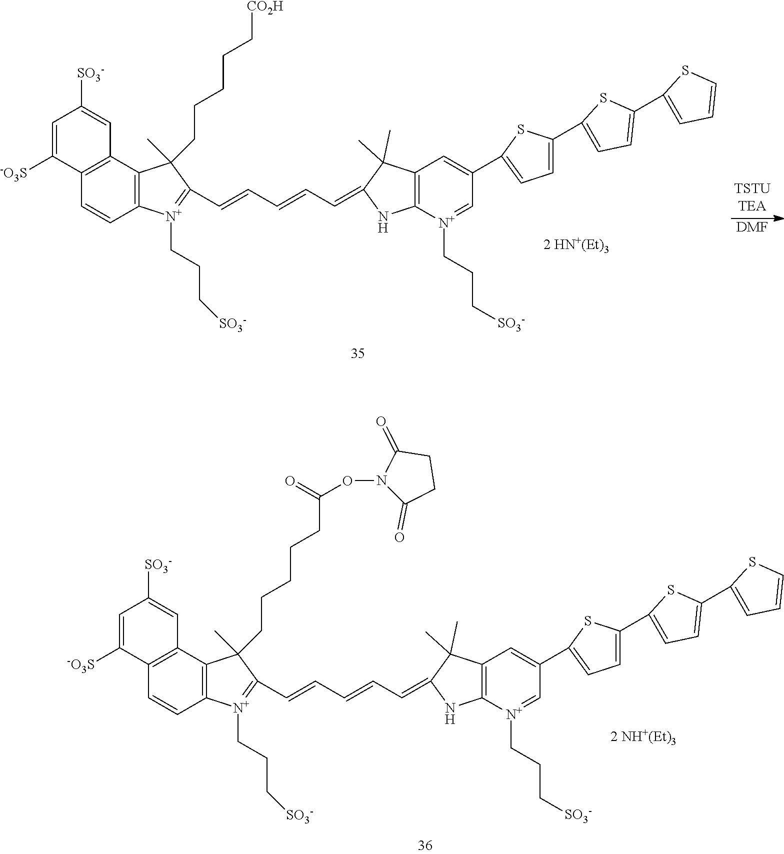

### Step 5: Synthesis of Compound 36

Compound 35 was synthesized in a manner similar to compound 30 to yield 9 mg (69% yield) of 36 as a green-blue solid. LCMS (negative) calculated 1253.18, observed 1253.17.

### Example 4: Synthesis of Compound 41

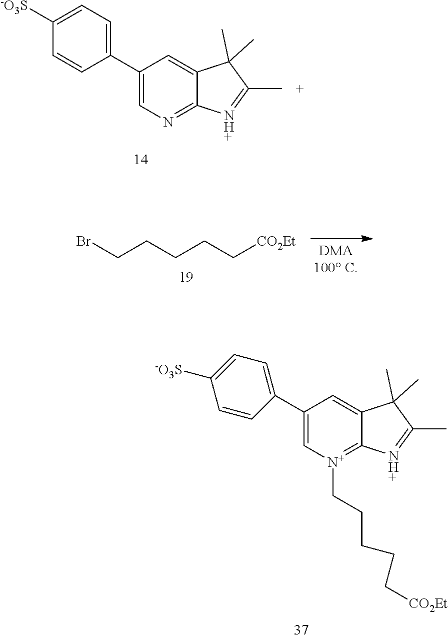

### Step 1: Synthesis of Compound 37

Compound 14 (1.20 g, 3079 mmol) and ethyl 6-bromohexanoate (19, 4.05 mL, 22.8 mmol) were mixed in 35 mL of dimethylacetamide and heated at 120° C. for 3.5 h. Upon cooling, excess EtOAc was added to the reaction solution to precipitate the product. The solid was washed with EtOAc and dried to give 1.59 g (78% yield) of a hygroscopic pale purple solid, 37. This was used without further purification. LCMS calculated 459.19, observed 459.19.

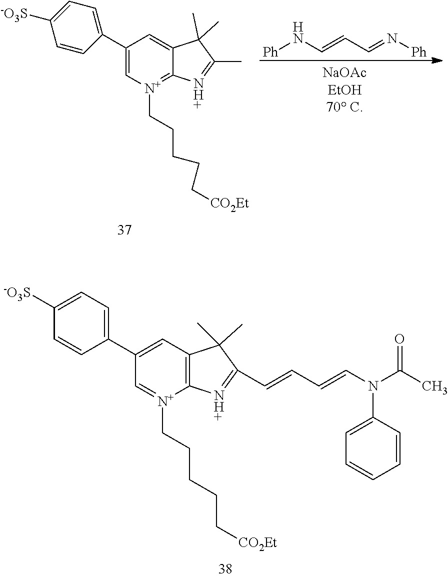

### Step 2: Synthesis of Compound 38

Compound 37 (1.47 g, 3.21 mmol), malonaldehyde dianilide HCl (3.32 g, 12.8 mmol), and sodium acetate (2.37 g, 28.9 mmol) were suspended in 125 mL of EtOH and heated at 70° C. for 3 h. Excess EtOAc (1.4 L) was added to precipitate a solid which was washed with EtOAc and dried to give 2.66 g of crude product as a purple-blue solid. This was used without further purification. LCMS calculated 588.25, observed 588.25.

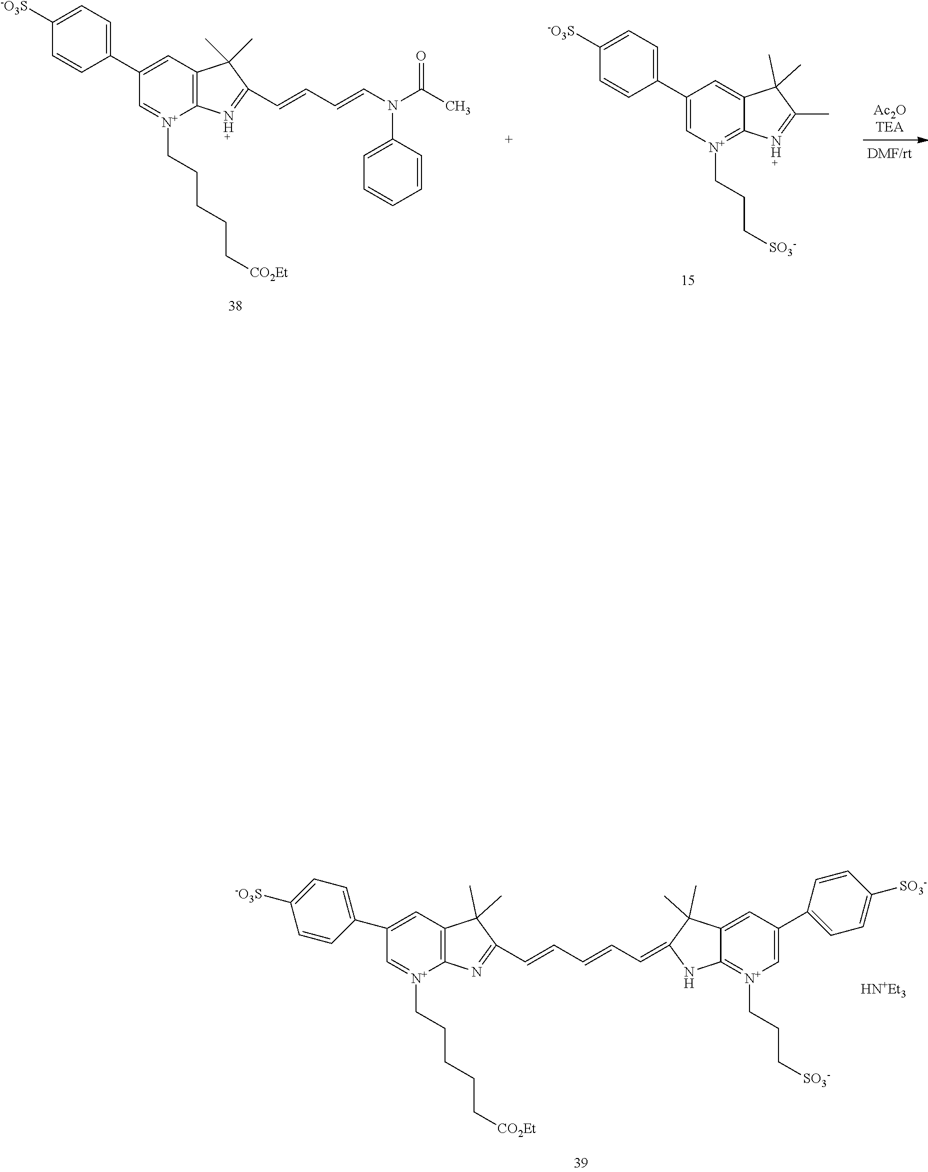

### Step 3: Synthesis of Compound 39

Compound 38 (2.66 g crude, expected 3.21 mmol) and compound 15 (1.00 g, 2.28 mmol) were dissolved in 125 mL DMF. To this was added acetic anhydride (1.21 mL, 12.8 mmol) and TEA (2.68 mL, 19.3 mmol) and the solution was stirred under N₂ at room temperature for 4 h. The solution was mixed with 1.4 L of EtOAc and the precipitated solid was collected, re-dissolved in 100 mL EtOH, re-precipitated with EtOAc, and dried. This was purified by column chromatography on RP media eluting with 0 to 35% MeOH/water, then re-purified with 40 to 60% MeOH/0.05 M TEAA. The combined concentrated solid was desalted on a pad of C₁₈ washing with water and eluting with MeOH to give 0.25 g (8% yield) of 39 as a dark blue solid. LCMS (negative) calculated 933.29, observed 933.28.

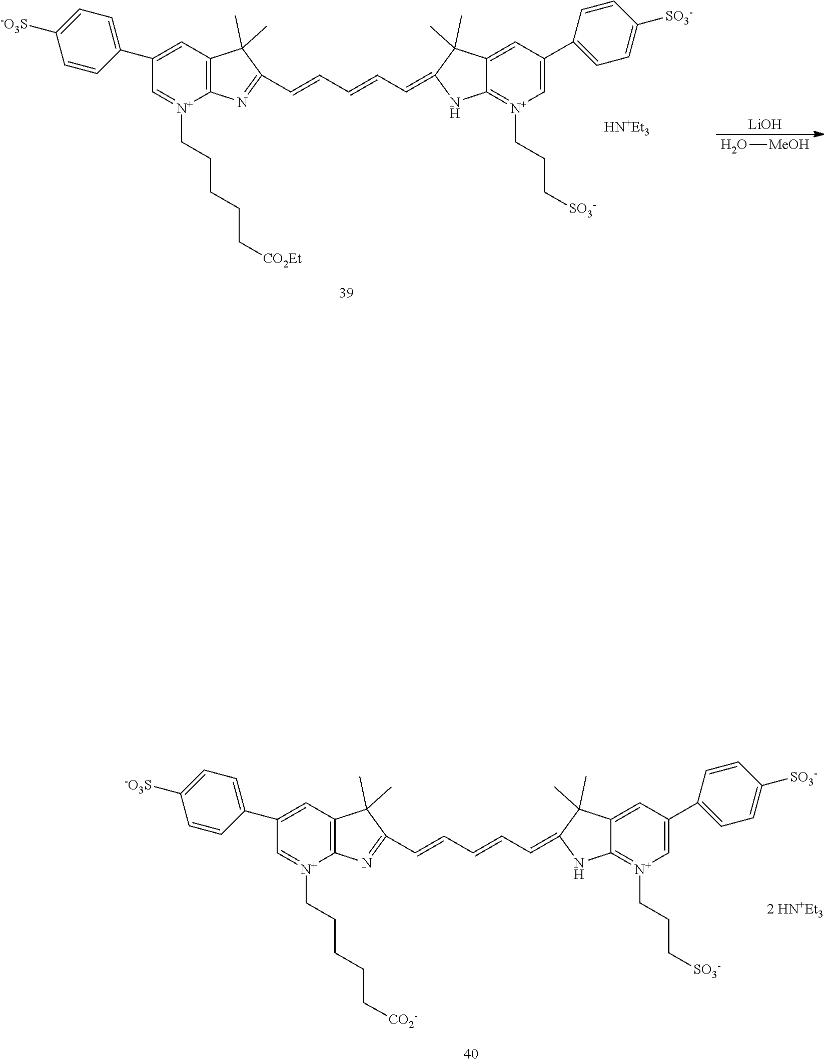

### Step 4: Synthesis of Compound 40

Compound 39 (0.25 g, 0.27 mmol) was mixed with 15 mL of MeOH and 15 mL of 1 M aqueous LiOH and stirred at room temperature for 1 h. 3 mL of acetic acid was added to neutralize and the MeOH removed by rotary evaporation. This was concentrated/desalted on a pad of C18 washing with water and eluting with MeOH. The concentrated residue was purified by column chromatography on RP media eluting with 40 to 60% MeOH/0.05 M TEAA. The concentrated, purified fractions were desalted on a pad of C18 washing with water and eluting with MeOH to give 0.21 g (88% yield) of 40 as dark blue solid. ¹H NMR (500 MHz, CDCl₃) δ 8.33 (d, 2H), 8.15 (t, 2H), 8.10 (s, 1H), 8.08 (s, 1), 7.95 (d, 4H), 7.79 (d, 4H), 6.60 (br s, 1H), 6.18 (d, 1H), 6.10 (d, 1H), 4.65 (t, 2H), 4.58 (t, 2H), 3.18 (q, 12H, 2 eq TEA), 2.99 (t, 2H), 2.50 (m, 2H), 2.32 (t, 2H), 2.05 (m, 2H), 1.75 (m, 2H), 1.53 (m, 2H), 1.50 (d, 12H), 1.30 (t, 18H, 2 eq TEA). LCMS (negative) calculated 906.26, observed 906.24. Excitation/emission in TE buffer 721 nm/743 nm.

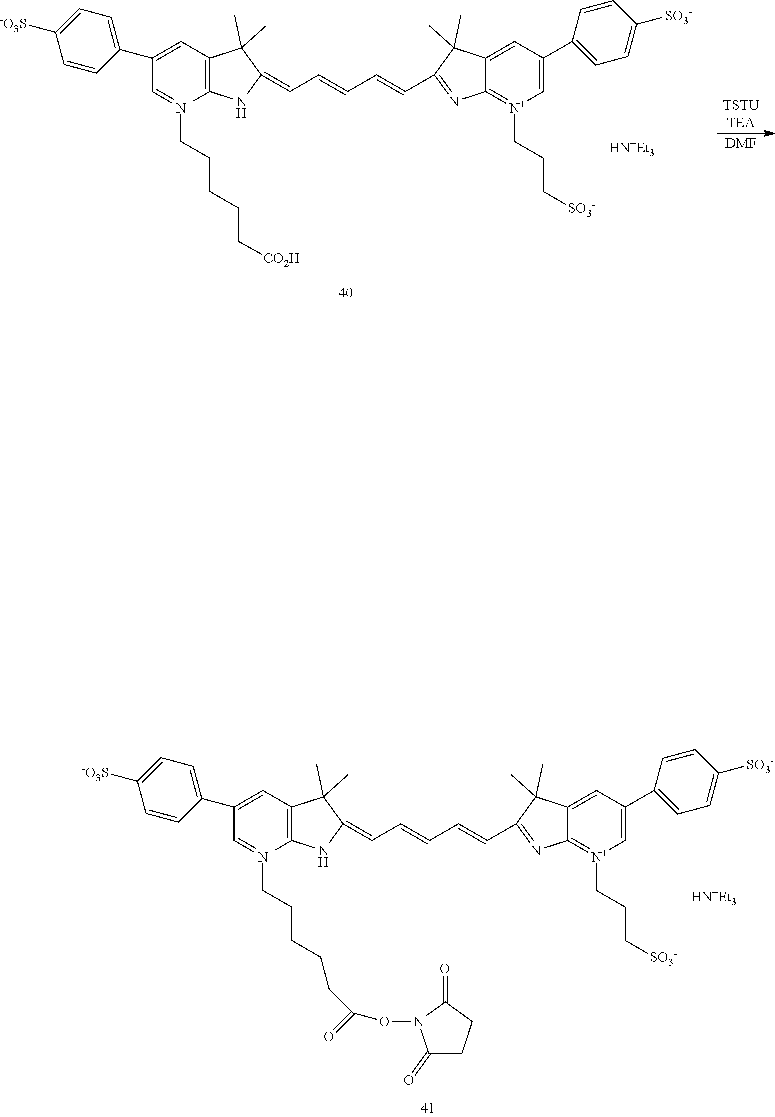

### Step 5: Synthesis of Compound 41

Compound 41 was synthesized in a manner similar to compound 36 using dried, degassed DMF as solvent to give 50 mg (82%) of 41 as a dark blue-black solid. LCMS (negative) calculated 1002.27, observed 1002.27. Compound 41 was coupled to an oligonucleotide and the extinction coefficient was measured in TE buffer using methods known to those skilled in the art (177,000 M⁻¹cm⁻¹).

### Example 5: Synthesis of Compound 43 for 2D NMR Spectroscopy Analysis

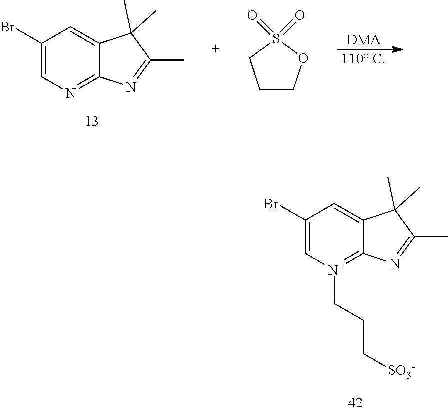

Compound 13 (100 mg, 0.418 mmol) was dissolved in 0.5 mL DMA and propane sultone (110 uL, 1.25 mmol) was added. This was heated at 110° C. for 45 min. The reaction solution was mixed with EtOAc to precipitate the product, which was then washed with EtOAc, Et₂O, and dried to give 181 mg of crude product 42 which was used without further purification.

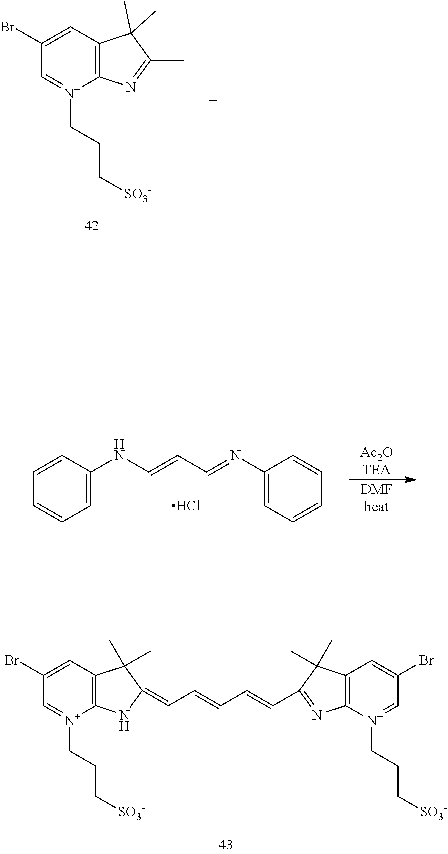

Crude compound 42 (151 mg crude, 0.42 mmol), malonaldehyde dianilide HCl (65 mg, 0.25 mmol), acetic anhydride (79 μL, 0.84 mmol), and TEA (176 μL, 1.3 mmol) were mixed and heated at 110° C. for 45 min. Upon cooling, the product was precipitated from the reaction solution by mixing with 150 mL EtOAc, and the solid was washed with EtOAc and dried. This was purified by column purification on RP media eluting with 30-60% MeOH/0.05 M TEAA. The concentrated product was desalted on a pad of C18 washing with water and eluting with MeOH to give 70 mg (44% yield) of 43 as a dark blue solid. ¹H NMR (500 MHz, CD₃OD) δ 8.21 (s, 2H), 8.13 (m, 2H), 7.80 (s, 2H), 6.60 (br s, 1H), 6.11 (d, 2H), 4.59 (t, 4H), 3.20 (q, 16H, TEA), 2.91 (t, 4H), 2.41 (m, 4H), 1.42 (s, 12H), 1.30 (t, 9H, TEA); ¹³C NMR (500 MHz, CD₃OD) δ 189.55, 163.87, 153.78, 147.53, 138.46, 134.89, 125.53, 110.05, 109.20, 53.52, 51.37, 49 (obscured by CH₃OD), 48.09, 26.32, 25.41, 9.42.

Two-dimensional NMR experiments were run on symmetrical cyanine compound (43) to verify the location of the propyl sulfonate group at position 7. The HSQC and HMBC spectra (data not shown) indicate that proton 12 is coupled to carbon 6, and that proton 12 is not coupled to carbon 2 or 3, verifying that the propyl sulfonate substituent is attached to the pyridyl nitrogen of compound (43).

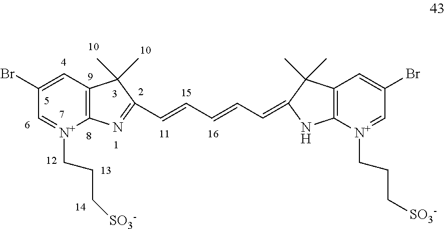

### Example 6: Synthesis of Compound 44 for 2D NMR Spectroscopy Analysis

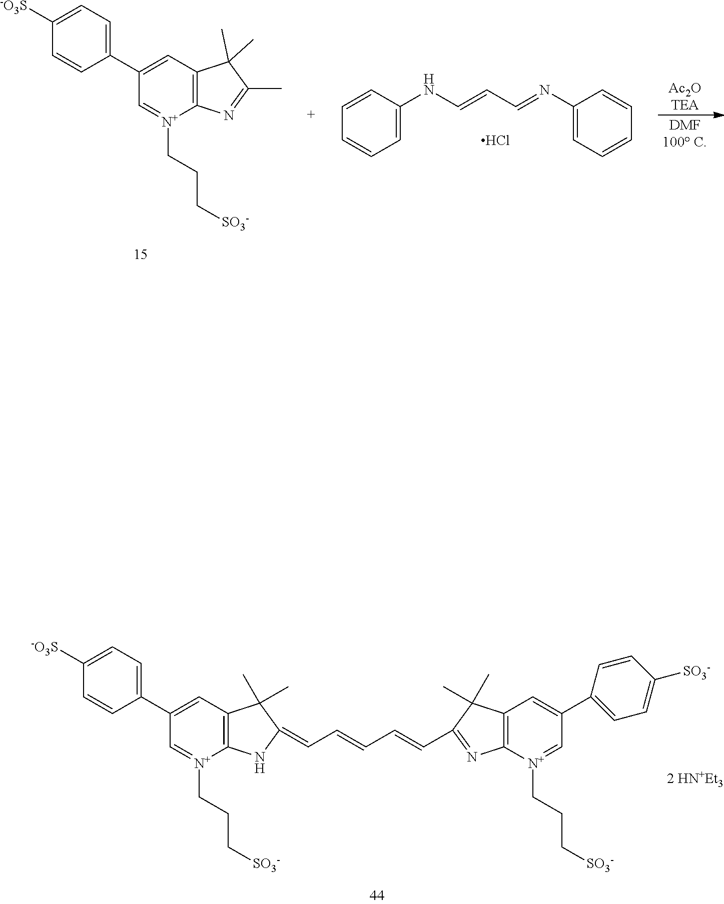

### Synthesis of Symmetrical Dye 44

Compound 15 (139 mg, 0.32 mmol, crude) and malonaldehyde dianilide HCl (50 mg, 0.19 mmol) were dissolved in 25 mL of dried, degassed DMF. Acetic anhydride (60 uL, 0.64 mmol) and TEA (134 uL, 0.96 mmol) were added and the solution was heated at 100° C. for 1 h. The reaction solution was mixed with 45 mL of EtOAc, and the solid precipitate collected was washed with EtOAc and dried. This was purified by column chromatography on RP media eluting with 30 to 40% MeOH/0.05 M TEAA. The purified pool was concentrated and desalted on a pad of C₁₈ washing with water and eluting with MeOH to give 39 mg (27% yield) of 44 as a black-purple solid. ¹H NMR (500 MHz, CD₃OD) δ 8.50 (s, 2H), 8.25 (m, 2H), 8.20 (s, 2H), 8.05 (d, 4H), 7.90 (d, 4H), 8.70 (br s, 1H), 6.25 (m, 2H), 4.82 (m, 4H), 3.30 (q, 18H, TEA), 3.05 (t, 4H), 2.60 (m, 4H), 1.60 (s, 12H), 1.40 (q, 27H, TEA); ¹³C NMR (500 MHz, CD₃OD) δ 163, 154, 147, 146, 139, 137, 129, 128, 54, 51, 50, 49, 27, 26, 10. LCMS (negative) calculated 914.20, observed 914.18.

Two-dimensional NMR experiments were run on symmetrical cyanine compound (44) to verify the location of the propyl sulfonate group at position 7. The HSQC and HMBC NMR spectra (data not shown) indicate that the proton at position 12 is coupled to carbon 6, verifying that the propyl sulfonate group is attached to the pyridyl nitrogen of compound (44).

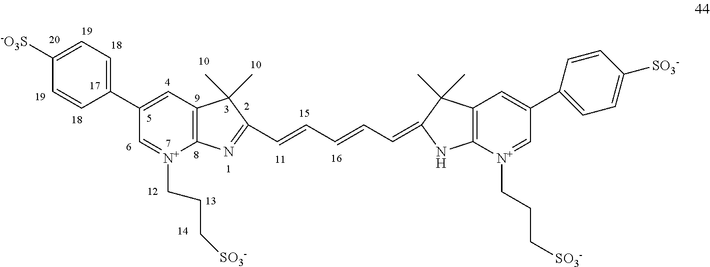

### Example 7: Synthesis of Compound 47

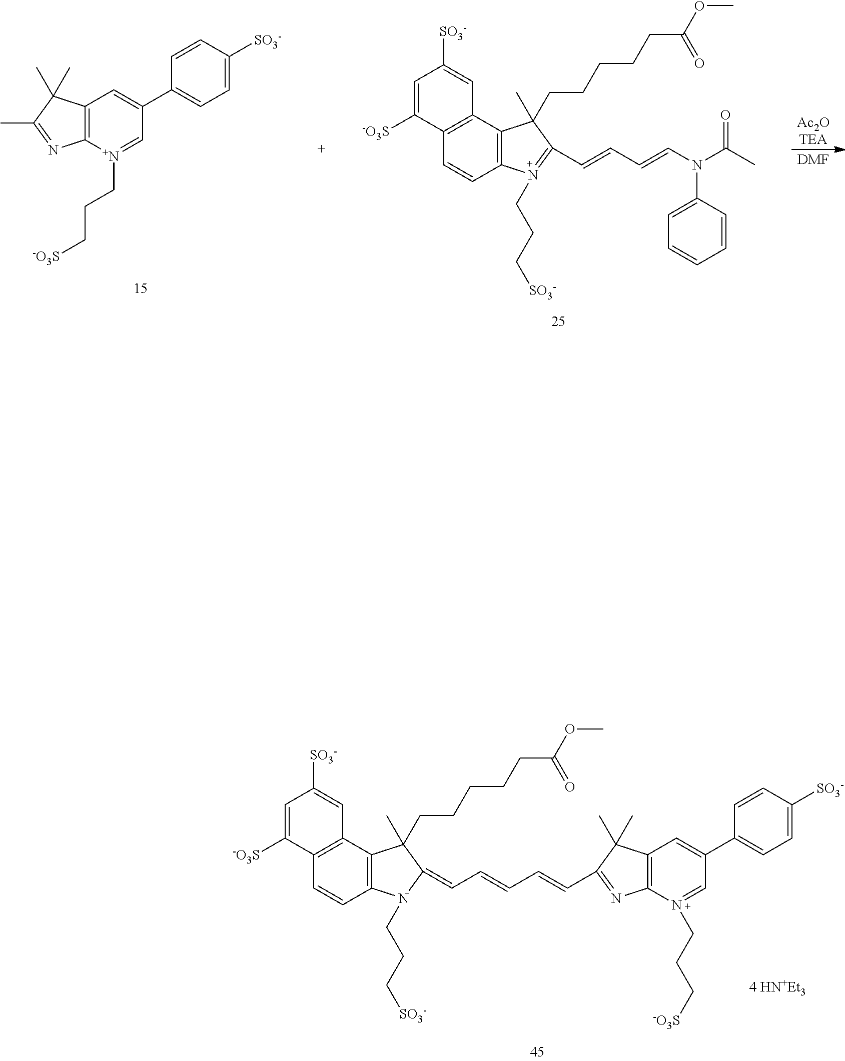

### Step 1: Synthesis of Compound 45

Compound 25 (169 mg, 0.153 mmol) and compound 15 (100 mg, 0.23 mmol, crude) were dissolved in 5 mL DMF. Acetic anhydride (73 uL) and TEA (160 uL) were added and the amber solution was stirred at room temperature for 4 h. The reaction solution was partially concentrated to reduce the DMF volume and then precipitated with EtOAc. The solid was washed with EtOAc and Et₂O, and dried. This was purified by reverse phase prep HPLC to yield 45 mg (20% yield) of 45 as a dark blue solid.

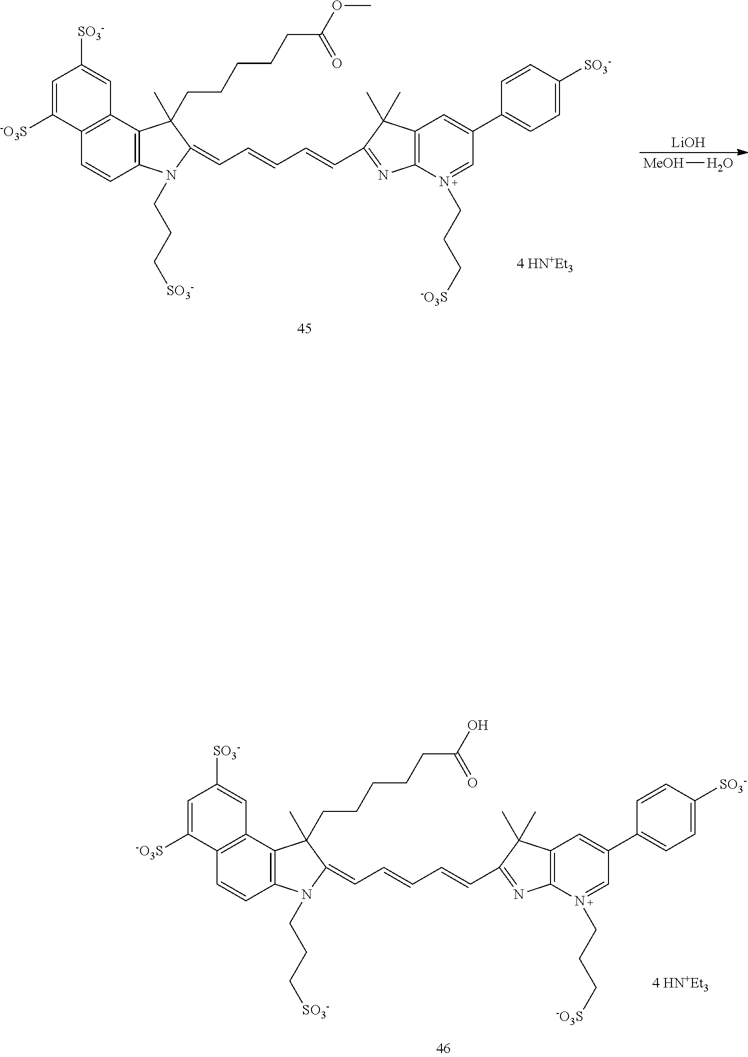

### Step 2: Synthesis of Compound 46

Compound 45 (40 mg, 0.027 mmol) was dissolved in 3 mL MeOH and mixed with 3 mL of 1 M aq LiOH. After stirring for 4 h at room temperature, 600 uL of AcOH was added to neutralize to pH 7. The solution was concentrated, and the residue was purified by reverse phase prep HPLC to yield 28 mg (70% yield) of 45 as a dark blue solid.

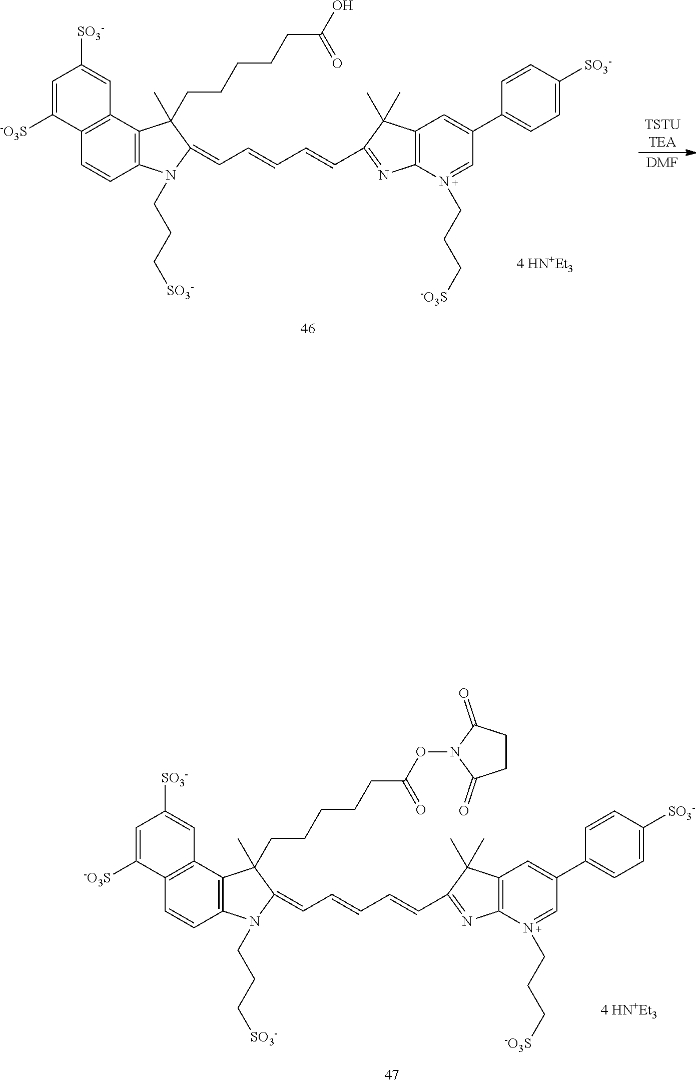

### Step 3: Synthesis of Compound 47

Compound 46 (15 mg, 0.0102 mmol) was dissolved in 500 uL of anhydrous DMF. TEA (10 μL) and N,N,N,N-tetramethyl-O—(N-succinimidyl)uranium tetrafluoroborate (TSTU, 4 mg, 0.0133 mmol) were added and the solution was stirred at room temperature for 1.5 h. The product was precipitated by adding excess EtOAc. The solid was washed with EtOAc and Et₂O, and dried under vacuum to yield 11.0 mg (69% yield) of 47 as a blue solid. UV-Vis (water): λabs=699 nm; λem=722 nm. Compound 47 was coupled to an oligonucleotide and the extinction coefficient was measured in TE buffer using methods known to those skilled in the art (225,000 M⁻¹cm⁻¹).

### Example 8: Synthesis of Compound 51

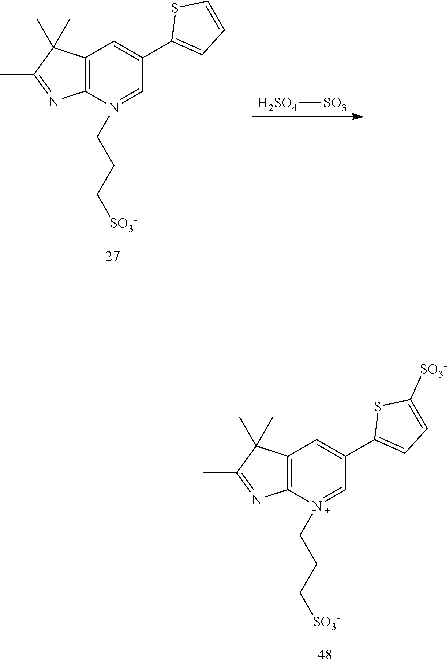

### Step 1: Synthesis of Compound 48

Compound 27 (300 mg) was dissolved in 5 mL concentrated sulfuric acid in a 50 mL round bottom flask and stirred in an ice bath. To the solution was added 1 mL of 30% fuming sulfuric acid and the mixture was stirred in ice bath for 10 minutes. The mixture was carefully poured into 100 mL cold ether in an ice bath under stirring. After stirring for 15 minutes, the ether layer was decanted, and the residue was washed with ether (100 mL) and ethyl acetate (100 mL). The residue was dissolved in 100 mL 10% trimethylamine in methanol and the solvent was evaporated. The residue was dissolved in water and lyophilized to dry to give about 400 mg of crude 48. The compound was used for next step without further purification.

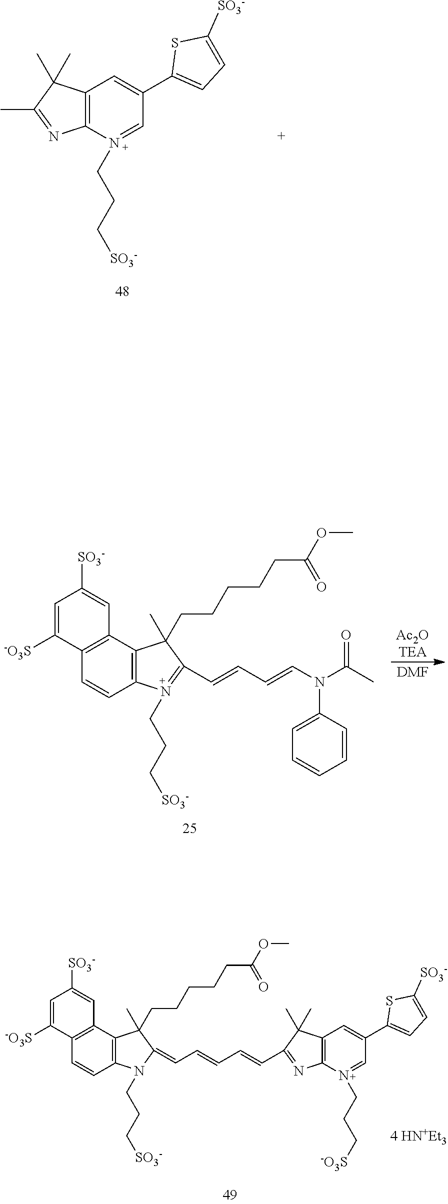

### Step 2: Synthesis of Compound 49

Compound 25 (120 mg, 0.108 mmol) and compound 48 (90 mg, 0.18 mmol, crude) were dissolved in 5 mL DMF. Acetic anhydride (50 μL) and TEA (110 μL) were added and the amber solution was stirred at room temperature for 4 h. The reaction solution was partially concentrated to reduce the DMF volume and then precipitated with EtOAc. The solid was washed with EtOAc and Et₂O, and dried. This was purified by reverse phase prep HPLC to yield 38 mg (25% yield) of 49 as a dark blue solid.

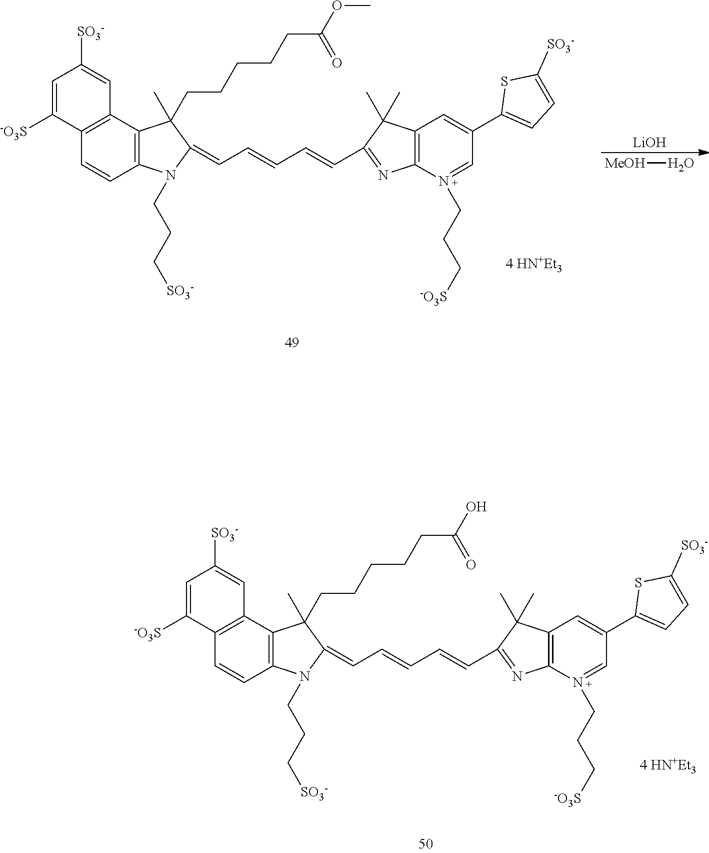

### Step 3: Synthesis of Compound 50

Compound 49 (30 mg, 0.020 mmol) was dissolved in 3 mL MeOH and mixed with 3 mL of 1 M aq LiOH. After stirring for 4 h at room temperature, 600 uL of AcOH was added to neutralize to pH 7. The solution was concentrated, and the residue was purified by reverse phase prep HPLC to yield 21 mg (70% yield) of 50 as a dark blue solid after lyophilization. Excitation/Emission 705 nm/733 nm.

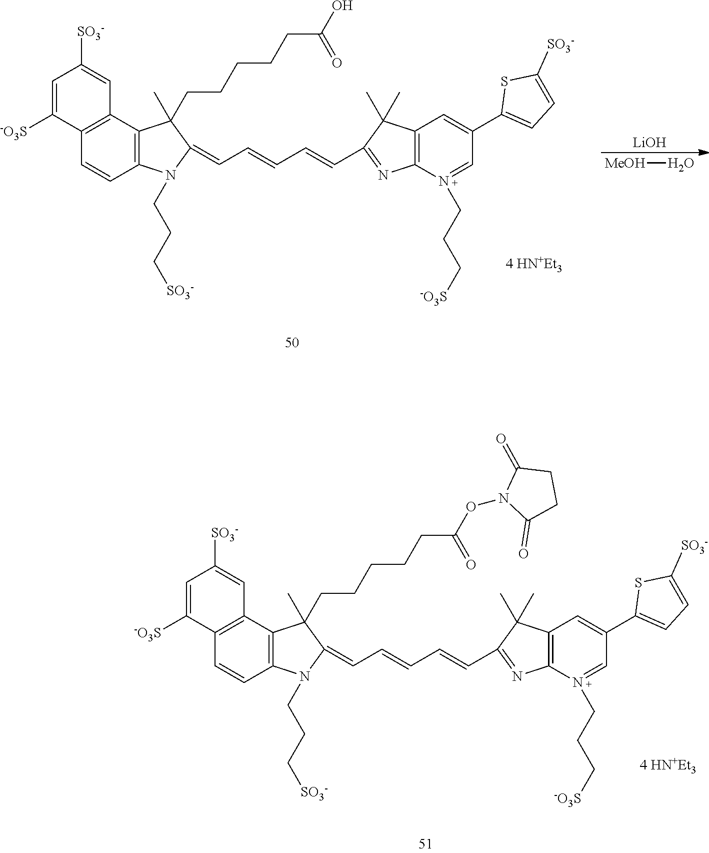

### Step 4: Synthesis of Compound 51

Compound 50 (10.0 mg, 0.0067 mmol) was dissolved in 500 uL of anhydrous DMF. TEA (5 uL) and N,N,N,N-tetramethyl-O—(N-succinimidyl)uranium tetrafluoroborate (TSTU, 3 mg, 0.0096 mmol) were added and the solution was stirred at room temperature for 1.5 h. The product was precipitated by adding excess EtOAc. The solid was washed with EtOAc and dried under vacuum to yield 8.0 mg (75% yield) of 51 as a blue solid. Excitation/Emission 705 nm/732 nm in water.

### Example 9: Synthesis of Compound 56

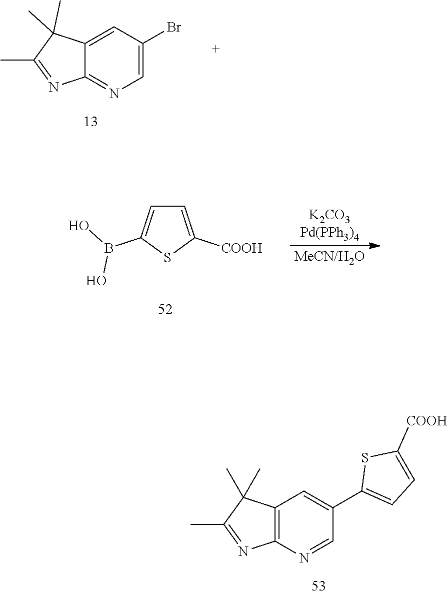 [SMILES: CC1=Nc2ncc(Br)cc2C1(C)C ; O=C(O)c1ccc(B(O)O)s1 ; "MeCN/H2O" ; CC1=Nc2ncc(-c3ccc(C(=O)O)s3)cc2C1(C)C ; "K2CO3Pd(PPh3)4"]

### Step 1: Synthesis of Compound 53

5-bromo-2,3,3-trimethyl-3H-pyrrolo[2,3-b]pyridine (13, 124 mg, 0.5 mmol), compound 52 (126 mg, 0.73 mmol), sodium carbonate (106 mg, 1.0 mmol), and tetrakis(triphenylphosphine) palladium (0) (66 mg, 0.118 mmol) were mixed in 10 mL acetonitrile-water (1:1) and degassed with argon for 10 min. The mixture was refluxed for 3 h. The mixture was dispersed in water, extracted into EtOAc three times, dried over anhydrous sodium sulfate, filtered, and concentrated to an amber-yellow oil. This was purified by column chromatography on silica gel eluting with 10-20% MeOH/CHCl₃ to give 107 mg (80% yield) of 53 as an amber solid.

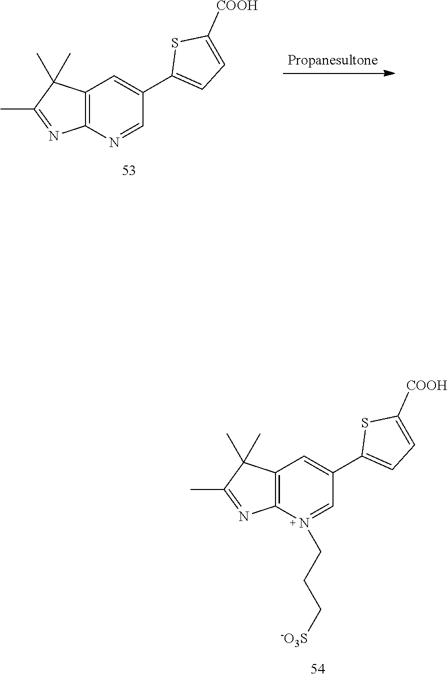

### Step 2: Synthesis of Compound 54

Compound 53 (107 mg, 0.374 mmol) and propane sultone (228 mg, 1.87 mmol) were dissolved in 4 mL of dimethylacetamide and heated at 110° C. for 1 h. Upon cooling, excess EtOAc was added and the product precipitated. The solid was washed with EtOAc and Et₂O and dried to yield 90 mg (60% yield) of 54 as a grey solid. The product was used without further purification.

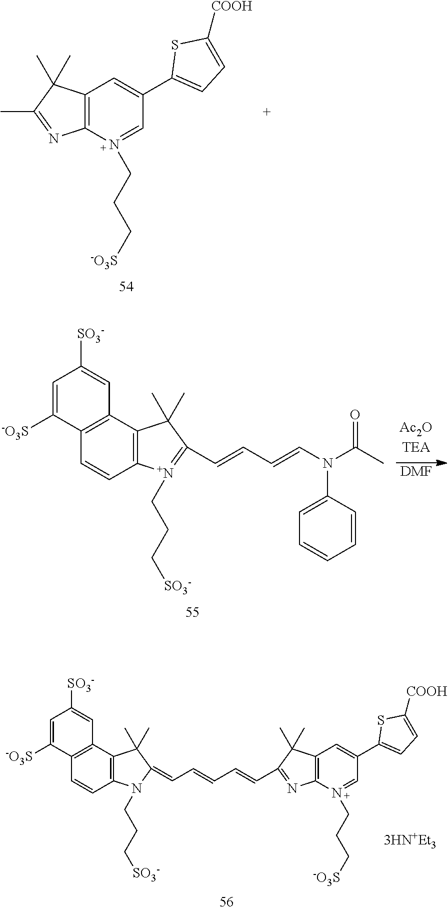

### Step 3 Synthesis of Compound 56

Compound 55 (50 mg, 0.075 mmol) and compound 54 (50 mg, 0.128 mmol, crude) were dissolved in 5 mL DMF. Acetic anhydride (73 μL) and TEA (160 μL) were added and the amber solution was stirred at room temperature for 4 h. The reaction solution was partially concentrated to reduce the DMF volume and then precipitated with EtOAc. The solid was washed with EtOAc and Et₂O, and dried. This was purified by reverse phase prep HPLC to yield 15 mg (16% yield) of 56 as a dark blue solid. Excitation/Emission 705 nm/732 nm in water.

### Example 10: Synthesis of Compound 61

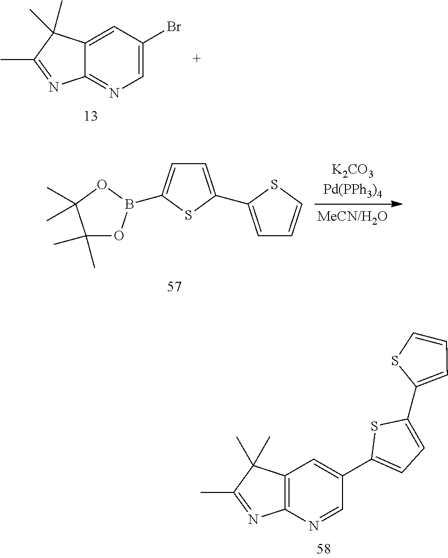 [SMILES: CC1=Nc2ncc(Br)cc2C1(C)C ; CC1(C)OB(c2ccc(-c3cccs3)s2)OC1(C)C ; CC1=Nc2ncc(-c3ccc(-c4cccs4)s3)cc2C1(C)C]

### Step 1: Synthesis of Compound 58

5-bromo-2,3,3-trimethyl-3H-pyrrolo[2,3-b]pyridine (13, 124 mg, 0.5 mmol), compound 57 (292 mg, 1.0 mmol), potassium carbonate (600 mg) and tetra-kis(triphenylphosphine) palladium (0) (120 mg, 0.247 mmol) were mixed in 10 mL dioxane and degassed with argon for 5 min. The mixture was heated at 80° C. overnight. The mixture was dispersed in water, extracted into EtOAc three times, dried over anhydrous sodium sulfate, filtered, and concentrated to an amber-yellow oil. This was purified by column chromatography on silica gel eluting with ethyl acetate to give 120 mg (73% yield) of 58 as an amber solid.

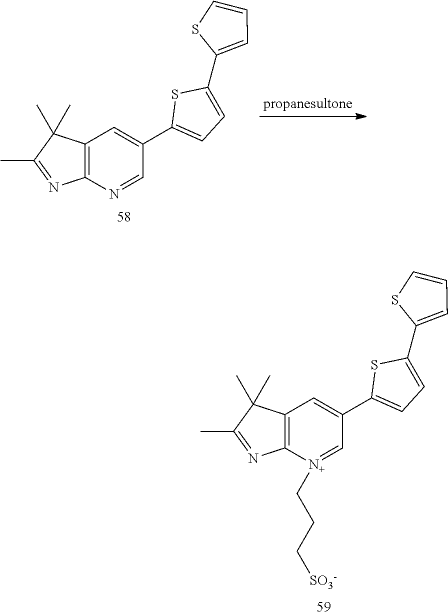

### Step 2: Synthesis of Compound 59

Compound 58 (106 mg, 0.327 mmol) and propane sultone (228 mg, 1.87 mmol) were dissolved in 3 mL of chlorobenzene and heated at 120° C. for 3 h. Upon cooling, excess EtOAc was added and the product was precipitated. The solid was washed with EtOAc and Et₂O and dried to yield 190 mg of crude 59 as a grey solid. The product was used without further purification.

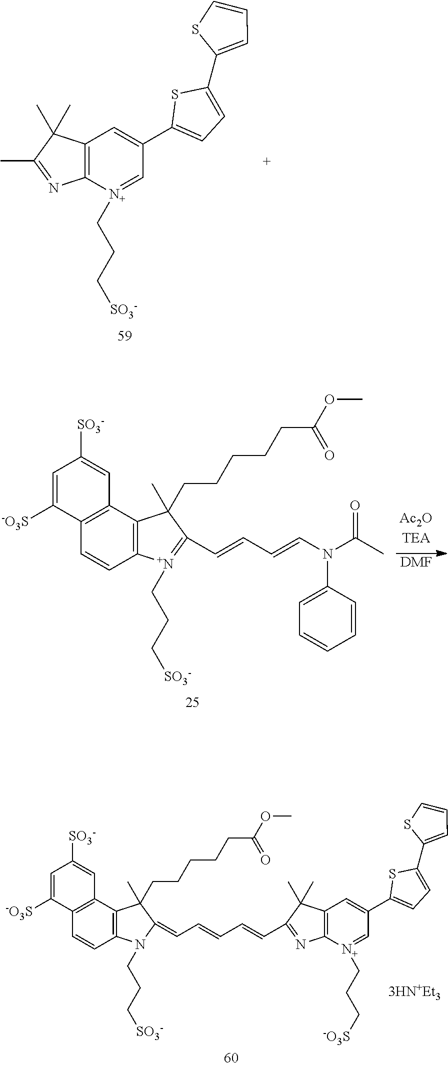

### Step 3 Synthesis of Compound 60

Compound 59 (27 mg, 0.06 mmol) and compound 25 (16 mg, 0.0189 mmol, crude) were dissolved in 1 mL DMF. Acetic anhydride (50 uL) and TEA (100 uL) were added and the amber solution stirred at room temperature for 6 h. The reaction solution was precipitated with EtOAc. The solid was washed with EtOAc and Et₂O, and dried. This was purified by reverse phase prep HPLC to yield 8 mg (30% yield) of 56 as a dark blue solid.

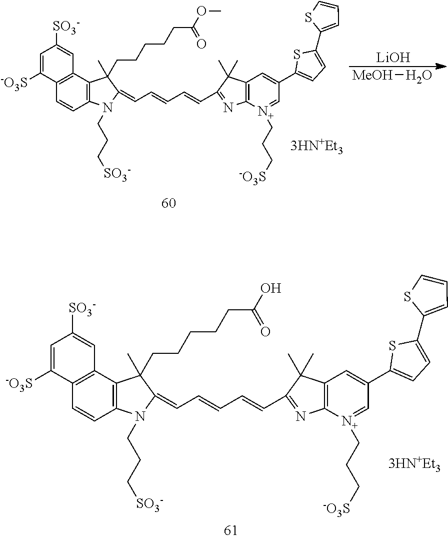

### Step 4 Synthesis of Compound 61

Compound 60 (8 mg, 0.0059 mmol) was dissolved in 1 mL MeOH and mixed with 1 mL of 1 M aq LiOH. After stirring for 1 h at room temperature, 200 uL of AcOH was added to neutralize to pH 7. The solution was concentrated, and the residue was purified by reverse phase prep HPLC to yield 7 mg (95% yield) of acid 61 as a dark blue solid after lyophilization. Excitation/Emission 711 nm/742 nm in water.

### Example 11: Synthesis of Compound 66

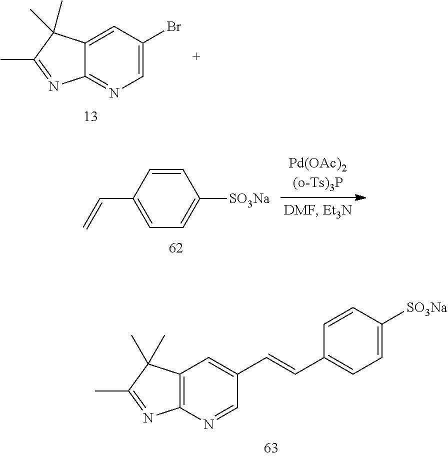

### Step 1: Synthesis of Compound 63

5-bromo-2,3,3-trimethyl-3H-pyrrolo[2,3-b]pyridine (13, 239 mg, 1.0 mmol), compound 62 (1.03 g, 5.0 mmol), Pd(OAc)₂ (120 mg, 0.247 mmol), (o-Ts)₃P (70 mg, 0.23 mmol) and triethylamine (1 mL) were mixed in 10 mL DMF and degassed with argon for 5 min. The mixture was heated at 110° C. for 1.5 hours. Upon cooling, the mixture was concentrated and purified by column chromatography on silica gel eluting with 20% MeOH/EtOAc to yield 224 mg (61% yield) of 63 as a pale-yellow solid.

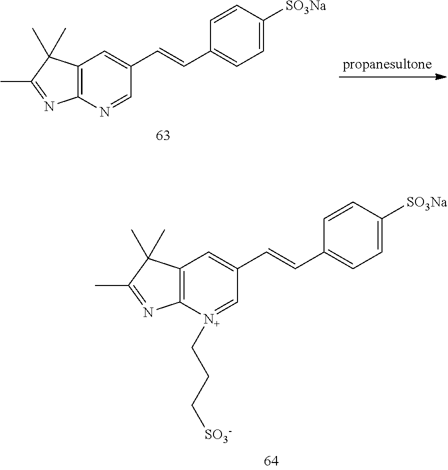

### Step 2: Synthesis of Compound 64

Compound 63 (124 mg, 0.339 mmol) and propane sultone (228 mg, 1.87 mmol) were dissolved in 3 mL of dimethylacetamide and heated at 120° C. for 3 h. Upon cooling, excess EtOAc was added and the product was precipitated. The solid was washed with EtOAc and Et₂O, and dried to yield 190 mg of crude 59 as a grey solid. The product was used without further purification.

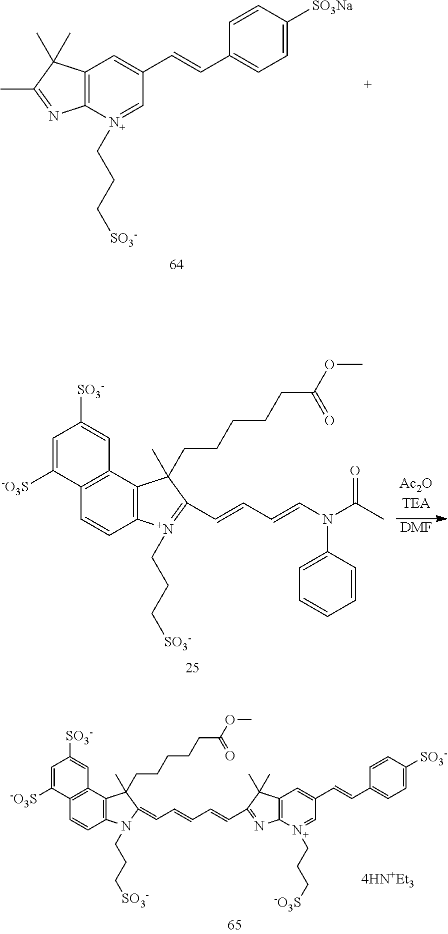

### Step 3 Synthesis of Compound 65

Compound 64 (50 mg, 0.103 mmol) and compound 25 (38 mg, 0.045 mmol, crude) were dissolved in 3 mL DMF. Acetic anhydride (200 μL) and TEA (400 μL) were added, and the amber solution stirred at room temperature for overnight. The reaction solution was precipitated with EtOAc. The solid was washed with EtOAc and Et₂O, and dried. This was purified by reverse phase prep HPLC to yield 18 mg (28% yield) of 65 as a dark blue solid.

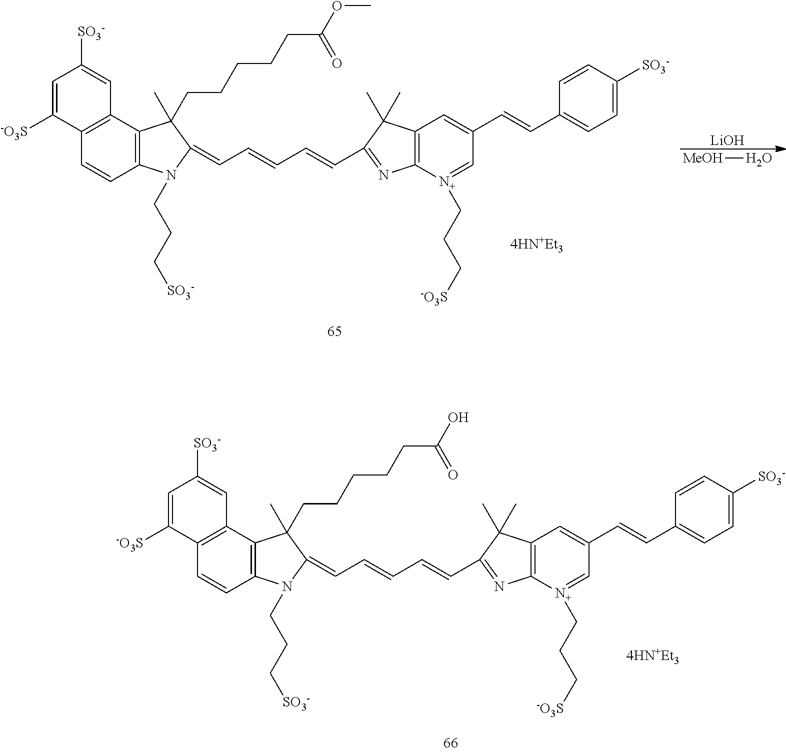

### Step 4 Synthesis of Compound 66

Compound 65 (18 mg, 0.012 mmol) was dissolved in 1 mL MeOH and mixed with 1 mL of 1 M aq LiOH. After stirring for 1 h at room temperature, 200 uL of AcOH was added to neutralize to pH 7. The solution was concentrated, and the residue was purified by reverse phase prep HPLC to yield 13 mg (72% yield) of acid 66 as a dark blue solid after lyophilization. Excitation/Emission 707 nm/741 nm in water.

### Example 12: Synthesis of Compound 72

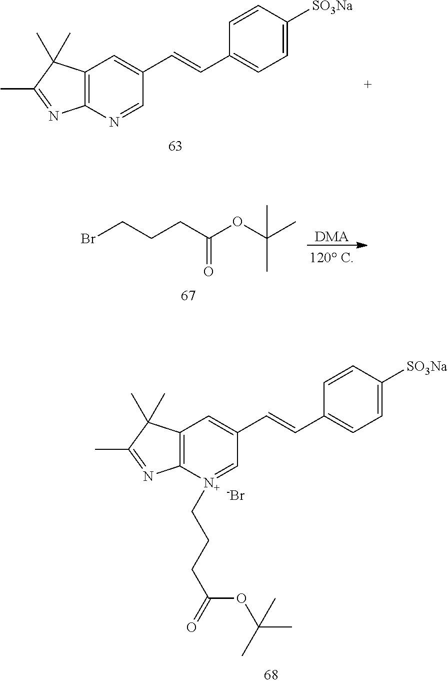

### Step 1: Synthesis of Compound 68

Compound 63 (124 mg, 0.339 mmol) and compound 67 (378 mg, 1.69 mmol) were dissolved in 2 mL of dimethylacetamide and heated at 120° C. for 6 h. Upon cooling, excess EtOAc was added and the product precipitated. The solid was washed with EtOAc, Et₂O, and dried to yield 167 mg of crude 59 as a grey solid. The product was used without further purification.

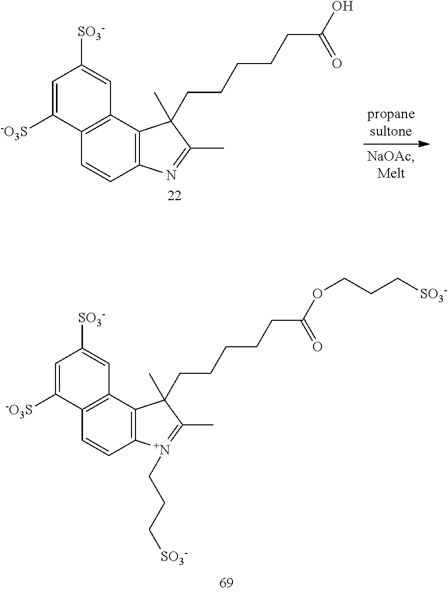

### Step 2: Synthesis of Compound 69

Compound 22 (1.35 g, 2.65 mmol), sodium acetate (0.63 g, 7.68 mmol), and propane sultone (1.35 mL, 15.4 mmol) were mixed well together and heated at 110° C. for 1 h. The cooled resin was dissolved in 15 mL of MeOH and precipitated from 150 mL EtOAc. The tan solid was washed with EtOAc, dried, and purified by column chromatography on RP media eluting with water to yield 1.41 g of 69 as a pale amber solid.

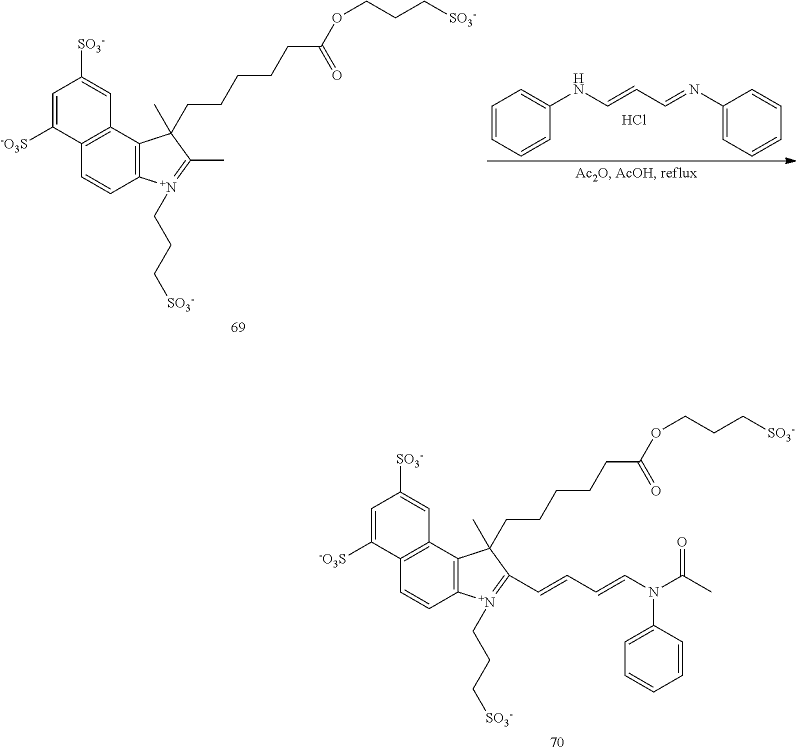

### Step 3: Synthesis of Compound 70

Compound 70 was prepared from Compound 69 using the procedure described for the synthesis of Compound 25 in step 4 of Example 2.

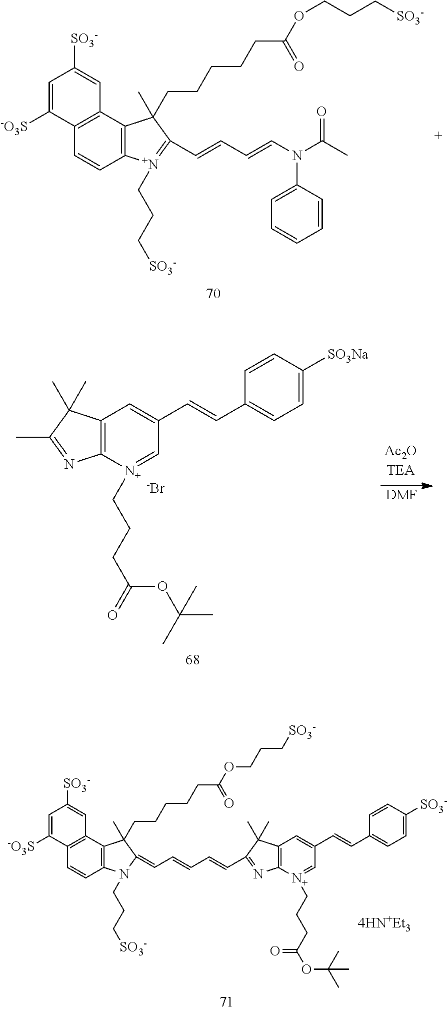

### Step 4: Synthesis of Compound 71

Compound 68 (50 mg) and compound 70 (38 mg, crude) were dissolved in 3 mL DMF. Acetic anhydride (200 μL) and TEA (400 μL) were added and the amber solution was stirred at room temperature for overnight. The reaction solution was precipitated with EtOAc. The solid was washed with EtOAc and Et₂O, and dried. This was purified by reverse phase prep HPLC to yield 18 mg (28% yield) of 65 as a dark blue solid.

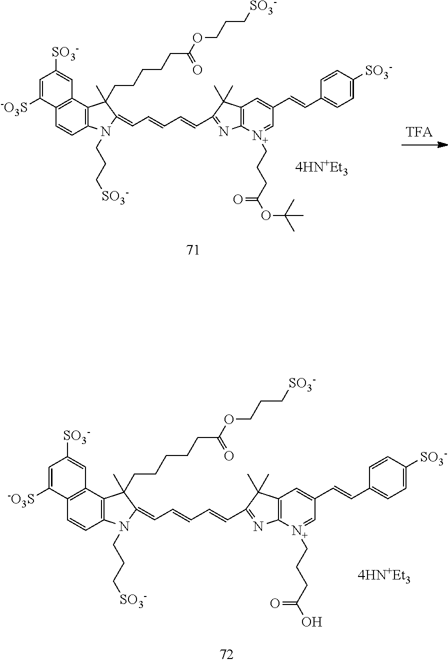

### Step 5: Synthesis of Compound 72

Compound 71 (18 mg) was dissolved in 2 mL TFA. After stirring for 1 h at room temperature, the solution was added into 90 mL ether. The precipitate was collected by filtration, washed with ether, and dried under vacuum. The solid was purified by reverse phase prep HPLC to yield 13 mg (65% yield) of bis-functioned acid 72 as a dark blue solid after lyophilization. Excitation/Emission 712 nm/740 nm in water.

### Example 13: Synthesis of Compound 79

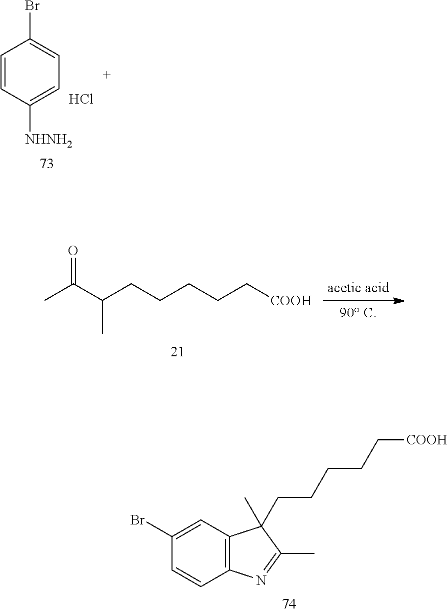 [SMILES: NNc1ccc(Br)cc1 ; Cl ; CC(=O)C(C)CCCCCC(=O)O ; CC1=Nc2ccc(Br)cc2C1(C)CCCCCC(=O)O]

### Step 1: Synthesis of Compound 74

Compound 73 (1.37 g, 6.13 mmol) and Compound 21 (1.37 g, 7.35 mmol) were suspended in HOAc (15 mL). The mixture was heated at 90° C. overnight. The reaction mixture was concentrated. The residue was dissolved in CHCl₃, washed with water three times, and dried over sodium sulfate. The crude was purified by column chromatography on silica gel eluting with hexanes-EtOAc-HOAc (70:30:3). After evaporation of the solvent, a pale-yellow solid was obtained (1.2 g).

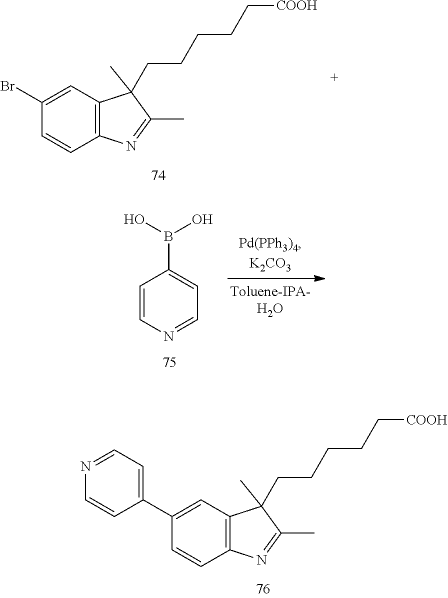 [SMILES: CC1=Nc2ccc(Br)cc2C1(C)CCCCCC(=O)O ; OB(O)c1ccncc1 ; CC1=Nc2ccc(-c3ccncc3)cc2C1(C)CCCCCC(=O)O]

### Step 2: Synthesis of Compound 76

Compound 74 (1.014 g, 3 mmol), compound 75 (480 mg, 3.9 mmol), potassium carbonate (2.5 g, 18.1 mmol) and tetra-kis(triphenylphosphine) palladium (0) (350 mg, 0.3 mmol) were mixed in a mixed solvent of toluene (8 mL)-IPA (4 mL)-water (2 mL) and degassed with argon for 5 min. The mixture was heated at 100° C. under argon overnight. After cooling, the mixture was partitioned in CHCl₃ (100 mL) and water (50 mL). The pH of the mixture was adjusted to pH 5-6 with 1 N HCl. The aqueous layer was extracted with CHCl₃ twice (2×40 mL). The combined organic layer was washed with brine, dried over anhydrous sodium sulfate, filtered, and concentrated to an amber-yellow oil. This was purified by column chromatography on silica gel eluting with 7% MeOH in CHCl₃ to give 820 mg (81% yield) of 76.

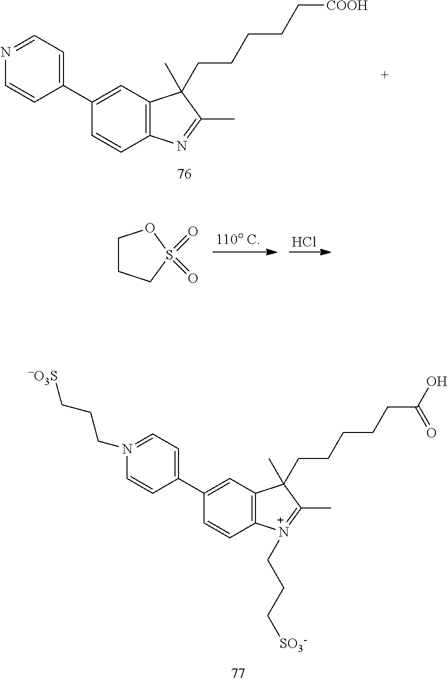

### Step 3. Synthesis of Compound 77

Compound 76 (800 mg, 2.38 mmol) and propane sultone (1.45 g, 11.88 mmol) were dissolved in 20 mL dry acetonitrile and stirred for 5 minutes. The solvent was evaporated to dry. The solid was heated at 110° C. for 1 hour. Acetonitrile (30 mL) was added to the residue and refluxed for 30 minutes. After cooling, the liquid was decanted, and the residue was washed with EtOAc (2×40 mL) and dried under vacuum. The residue was dissolved in a mixture of water (4 mL) and concentrated HCl (1 mL). The mixture was heated at 40° C. for 4 hours. After evaporation of the solvent, the solid was dissolved in 20 mL water and lyophilized overnight to give 2 g of dark solid. The compound was used for next step without further purification.

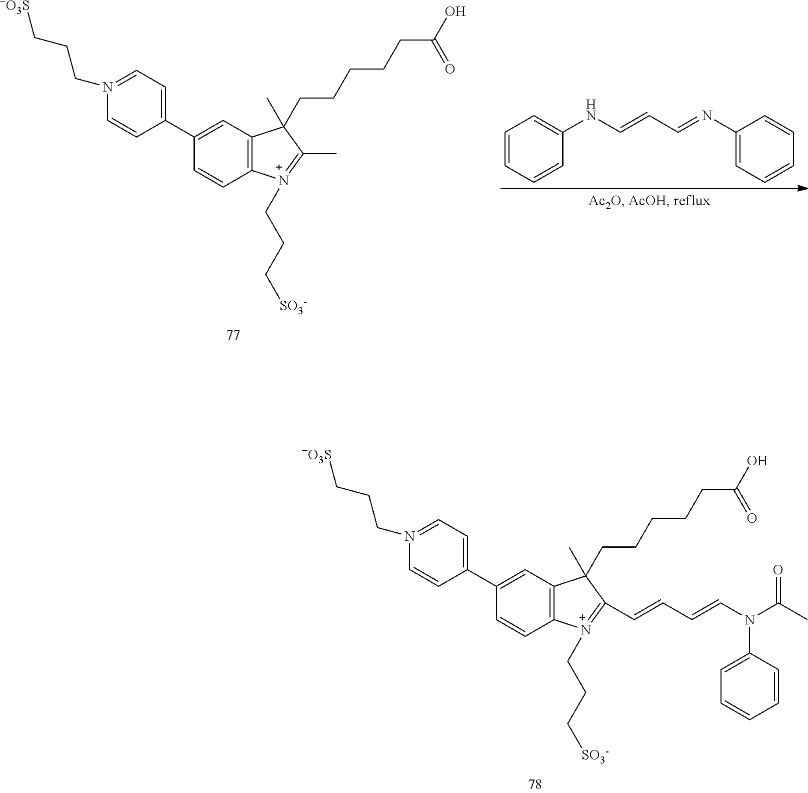

### Step 4: Synthesis of Compound 78

Compound 78 was prepared from Compound 77 using the procedure described for the synthesis of Compound 25 in step 4 of Example 2.

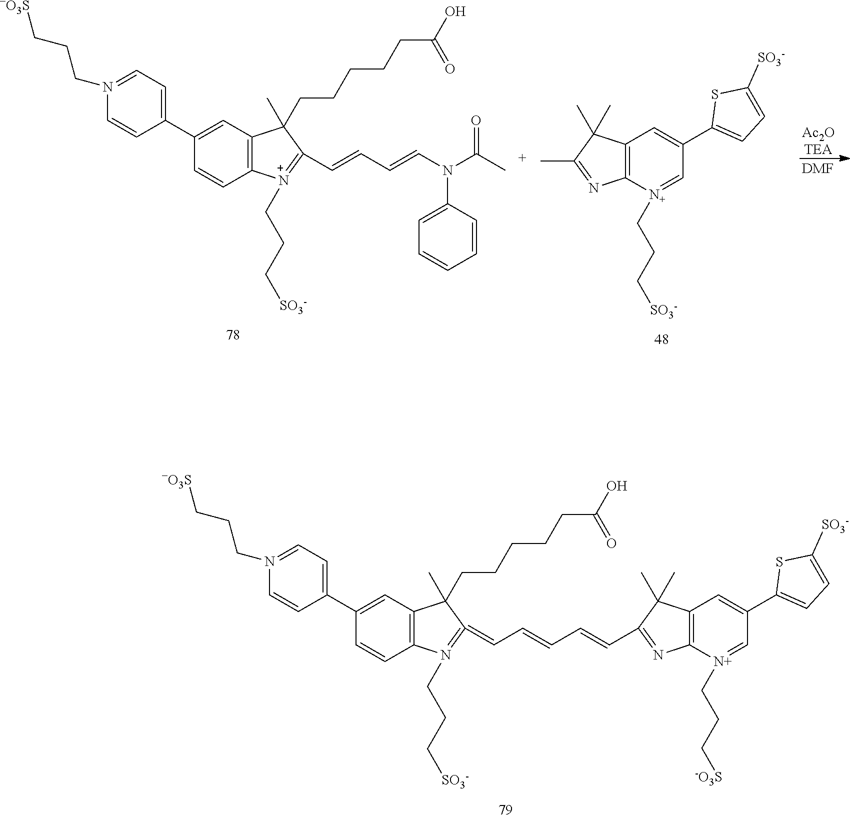

### Step 4: Synthesis of Compound 79

Compound 79 was prepared from Compound 78 and Compound 48 using the procedure described for the synthesis of Compound 49 in step 2 of Example 8. Excitation/Emission 702 nm/732 nm in water.

### Example 14: Synthesis of Compound 81

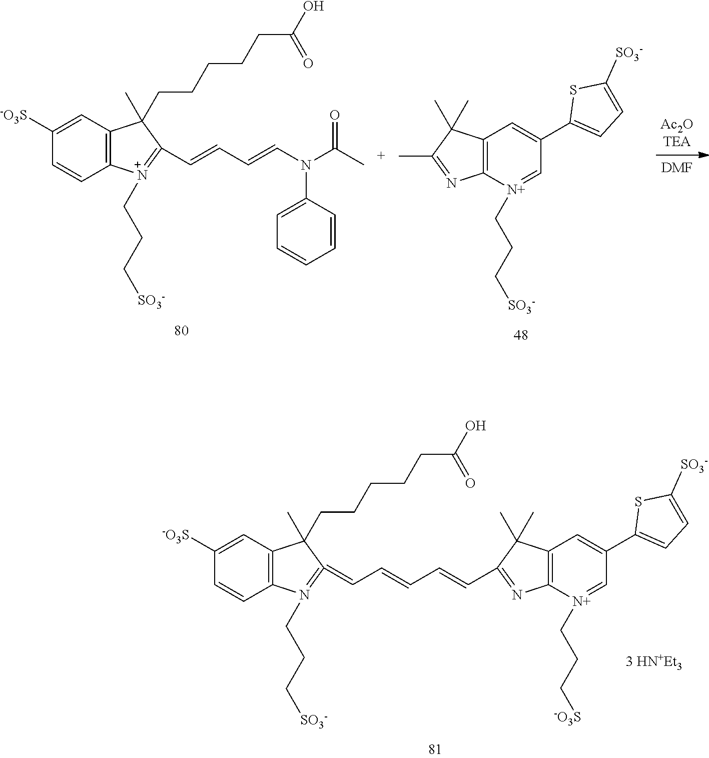

### Step 1: Synthesis of Compound 81

Compound 80 was synthesized using the procedure reported in U.S. Pat. No. 7,790,893. Compound 81 was prepared from Compound 80 and Compound 48 using the procedure described for the synthesis of Compound 49 in step 2 of Example 8. Excitation/Emission 685 nm/711 nm in water.

### Example 15: Synthesis of Compound 86

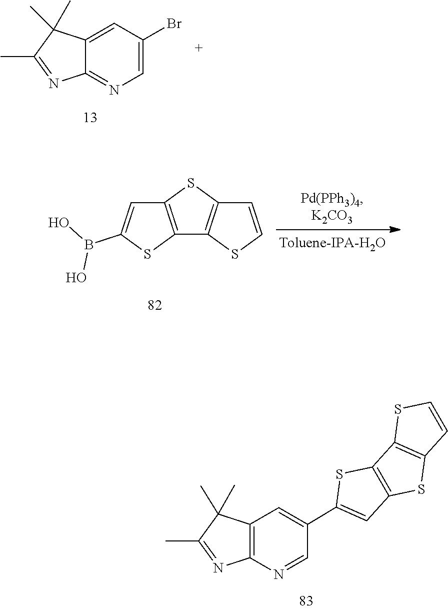 [SMILES: CC1=Nc2ncc(Br)cc2C1(C)C ; OB(O)c1cc2sc3ccsc3c2s1 ; CC1=Nc2ncc(-c3cc4sc5ccsc5c4s3)cc2C1(C)C]

### Step 1: Synthesis of Compound 83

Compound 13 (60 mg, 0.25 mmol), compound 82 (120 mg, 0.5 mmol), potassium carbonate (220 mg) and tetrakis(triphenylphosphine) palladium (0) (60 mg, 0.052 mmol) were mixed in a mixed solvent of toluene (4 mL)-IPA (2 mL)-water (1.5 mL) and degassed with argon for 5 min. The mixture was heated at 100° C. under argon for 9 hours. After cooling, the mixture was poured into water (100 mL). The mixture was extracted with EtOAc (3×50 mL). The combined organic layer was washed with brine, dried over anhydrous sodium sulfate, filtered, and concentrated to an amber-yellow oil. This was purified by column chromatography on silica gel eluting with EtOAc to give 60 mg (68% yield) of 83.

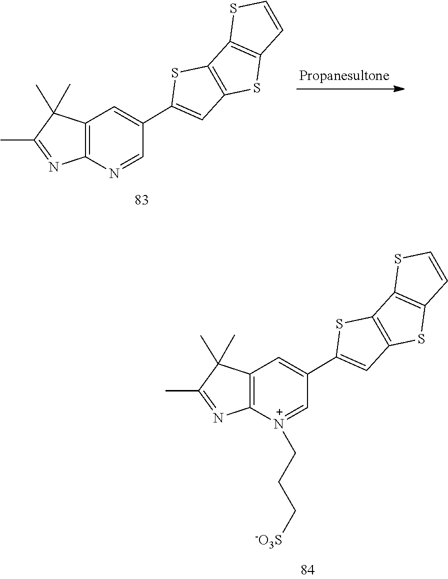

### Step 2: Synthesis of Compound 84

Compound 84 was prepared from Compound 83 using the procedure described for the synthesis of Compound 59 in step 2 of Example 10.

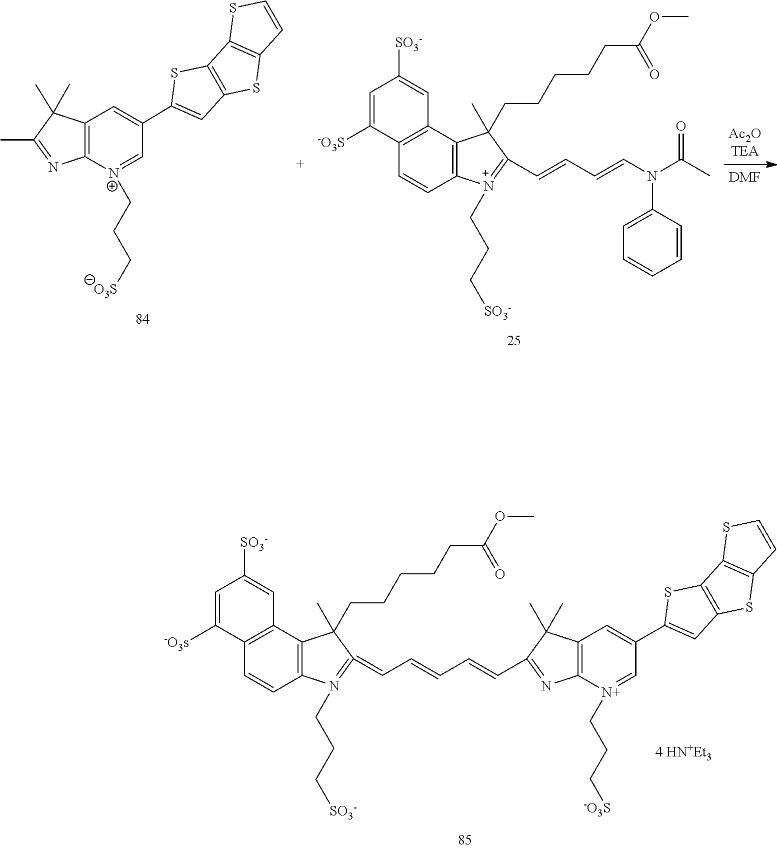

### Step 3: Synthesis of Compound 85

Compound 85 was prepared from Compound 84 and Compound 25 using the procedure described for the synthesis of Compound 60 in step 3 of Example 10.

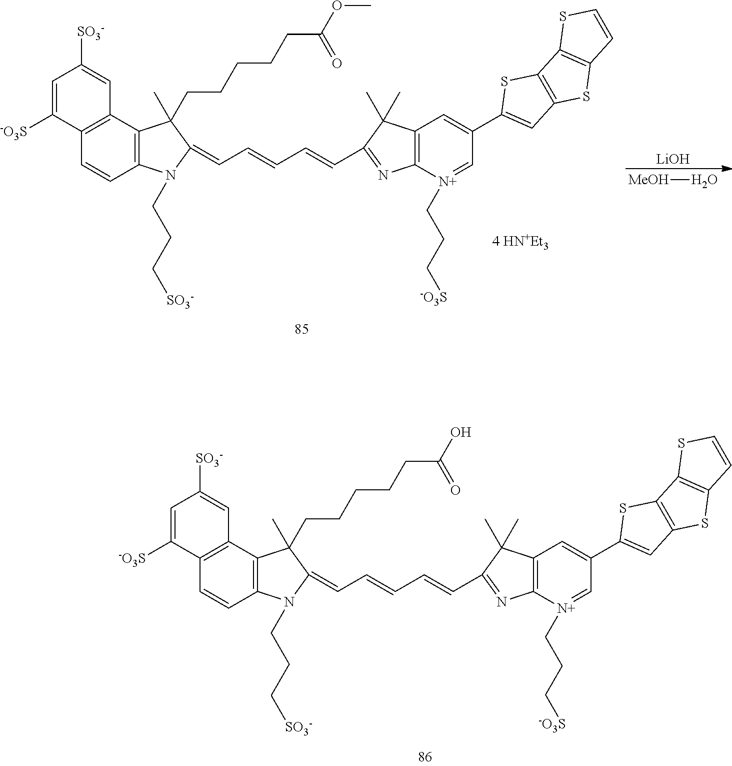

### Step 4: Synthesis of Compound 86

Compound 86 was prepared from Compound 85 using the procedure described for the synthesis of Compound 61 in step 4 of Example 10. Excitation/Emission 713 nm/742 nm in water.

### Example 16: Synthesis of Compound 91

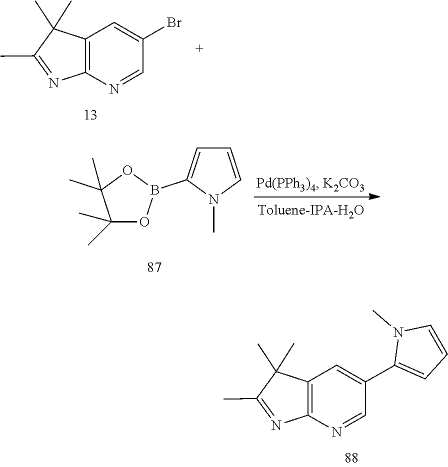 [SMILES: CC1=Nc2ncc(Br)cc2C1(C)C ; Cn1cccc1B1OC(C)(C)C(C)(C)O1 ; CC1=Nc2ncc(-c3cccn3C)cc2C1(C)C]

### Step 1: Synthesis of Compound 88

Compound 88 was prepared from Compound 13 and Compound 87 using the procedure described for the synthesis of Compound 83 in step 1 of Example 15.

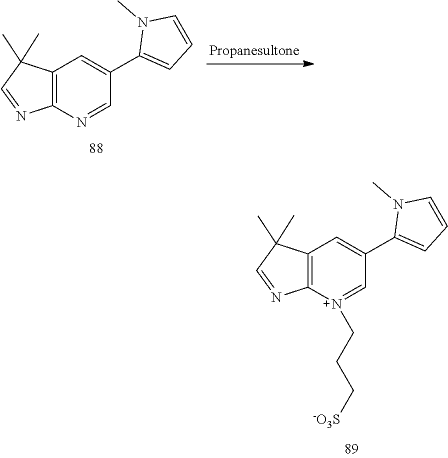

### Step 2: Synthesis of Compound 89

Compound 89 was prepared from Compound 88 using the procedure described for the synthesis of Compound 59 in step 2 of Example 10.

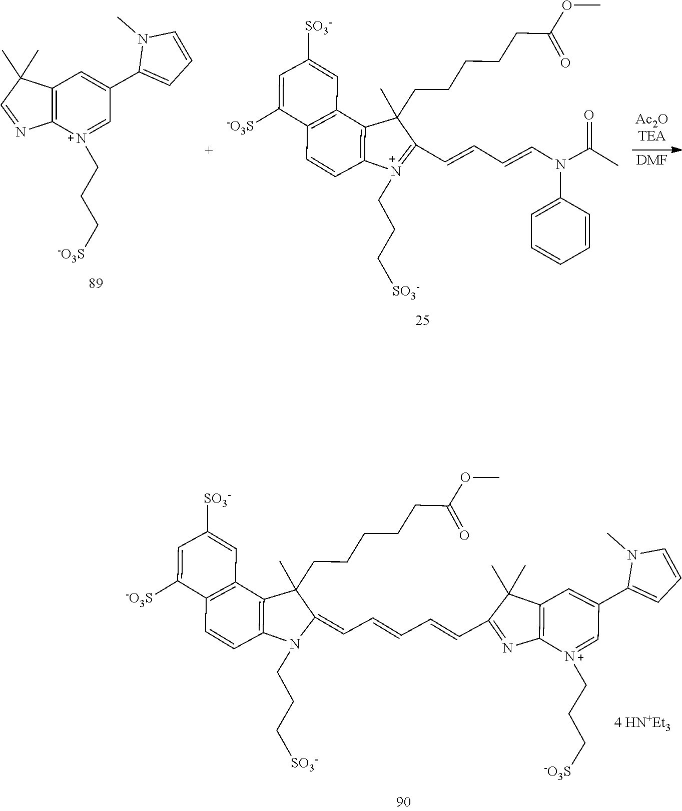

### Step 3: Synthesis of Compound 90

Compound 90 was prepared from Compound 89 and Compound 25 using the procedure described for the synthesis of Compound 60 in step 3 of Example 10.

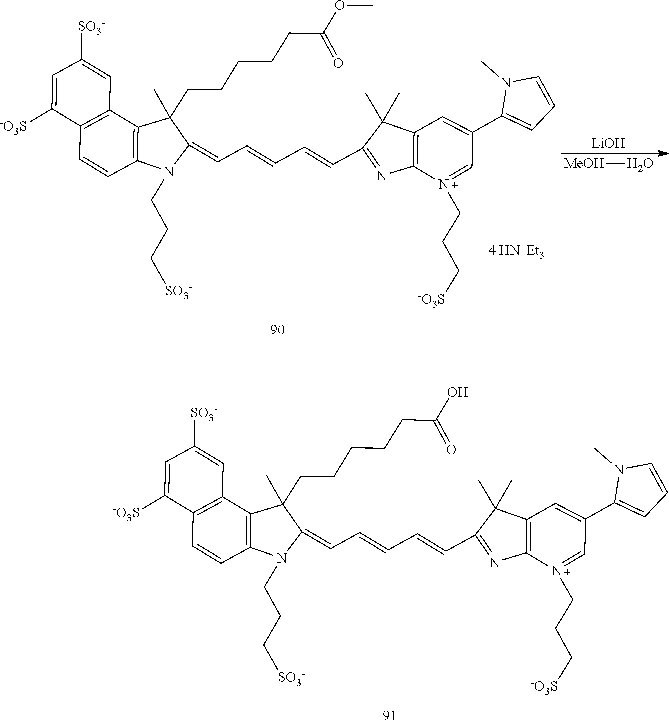

### Step 4: Synthesis of Compound 91

Compound 91 was prepared from Compound 90 using the procedure described for the synthesis of Compound 61 in step 4 of Example 10. Excitation/Emission 696 nm/726 nm in water.

### Example 17: Synthesis of Compound 92

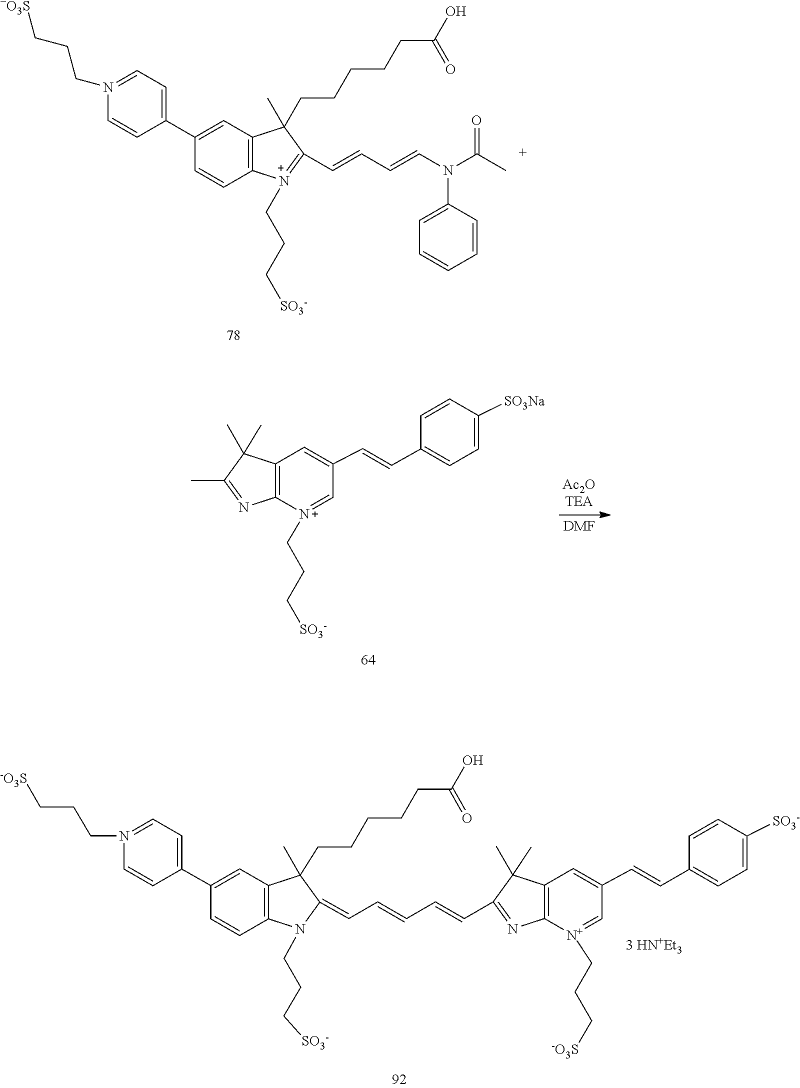

### Step 1: Synthesis of Compound 92

Compound 92 was prepared from Compound 78 and Compound 64 using the procedure described for the synthesis of Compound 60 in step 3 of Example 10. Excitation/Emission 707 nm/741 nm in water.

### Example 18: Synthesis of Compound 96

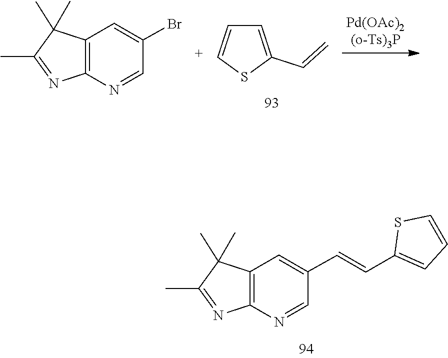

### Step 1: Synthesis of Compound 94

Compound 94 was prepared from Compound 13 and Compound 93 using the procedure described for the synthesis of Compound 63 in step 1 of Example 11.

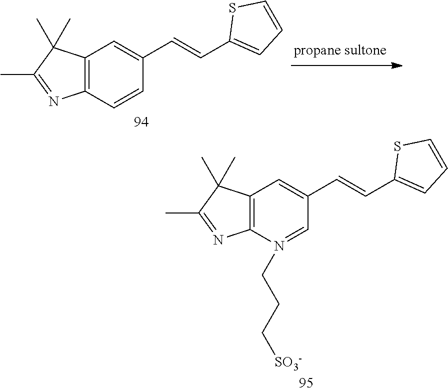

### Step 2: Synthesis of Compound 95

Compound 89 was prepared from Compound 94 using the procedure described for the synthesis of Compound 59 in step 2 of Example 10.

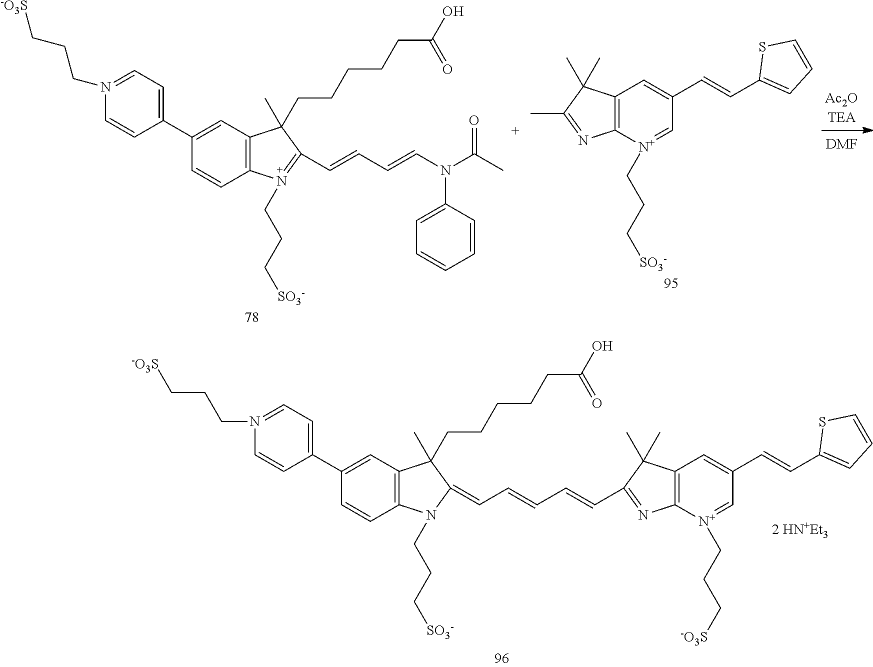

### Step 1: Synthesis of Compound 96

Compound 96 was prepared from Compound 78 and Compound 95 using the procedure described for the synthesis of Compound 49 in step 2 of Example 8. Excitation/Emission 709 nm/741 nm in water.

### Example 19: Synthesis of Compound 104

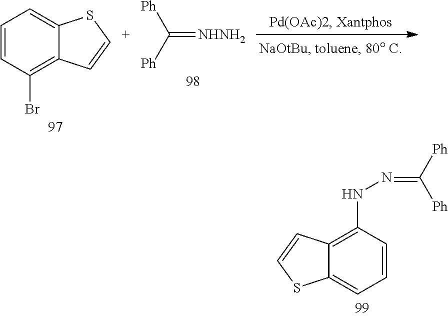

### Step 1: Synthesis of Compound 99

An oven-dried 3-neck 50 mL flask was evacuated and backfilled with argon, and charged with Pd(OAc)₂ (8 mg, 0.04 mmol), Xantphos (24 mg, 0.044 mmol) and anhydrous toluene (1 mL). The mixture was stirred at room temperature for 5 minutes. Compound 97 (2.13 g, 10 mmol) and Compound 98 (1.96 g, 10 mmol) were added to the flask, followed by an additional portion of anhydrous toluene (9 mL). The flask was evacuated and backfilled with argon twice. The solution was heated at 80° C. until completion of the reaction as determined by TLC (silica gel plate, eluting with 30% EtOAc in hexanes). After cooling to room temperature, ether (15 mL) was added to the mixture and the mixture was filtered through a pad of silica gel (1 inch). The silica gel was rinsed with ether (15 mL) and EtOAc (15 mL). The filtrate was concentrated, and the residue was recrystallized from methanol to give a yellow solid (2.9 g, 88%).

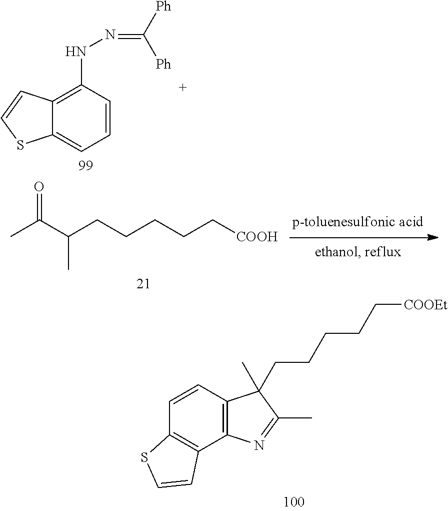

### Step 2: Synthesis of Compound 100

Compound 99 (984 mg, 3 mmol), Compound 21 (837 mg, 4.5 mmol), and p-TsOH monohydrate (2.28 g, 12 mmol) were dissolved in ethanol (25 mL). The solution was refluxed for 20 hours. TLC indicated the completion of the reaction. After cooling, the solvent was evaporated, 100 mL of water was added, and the mixture was extracted with EtOAc (3×100 mL). The combined organic layer was washed with brine and dried over anhydrous sodium sulfate, filtered, and concentrated to an amber-yellow oil. This was purified by column chromatography on silica gel eluting with EtOAc-hexanes to give 720 mg (70% yield) of 100.

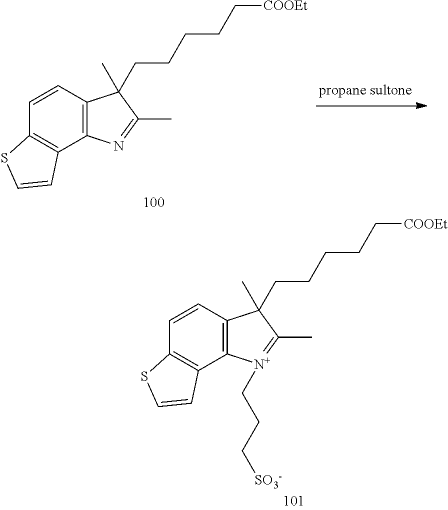

### Step 3: Synthesis of Compound 101

Compound 101 was prepared from Compound 100 using the procedure described for the synthesis of Compound 59 in step 2 of Example 10.

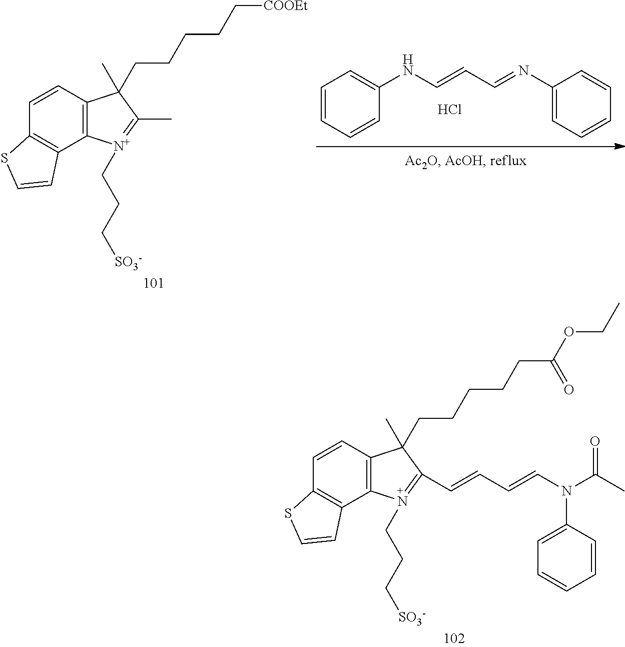

### Step 4: Synthesis of Compound 102

Compound 102 was prepared from Compound 101 using the procedure described for the synthesis of Compound 25 in step 4 of Example 2.

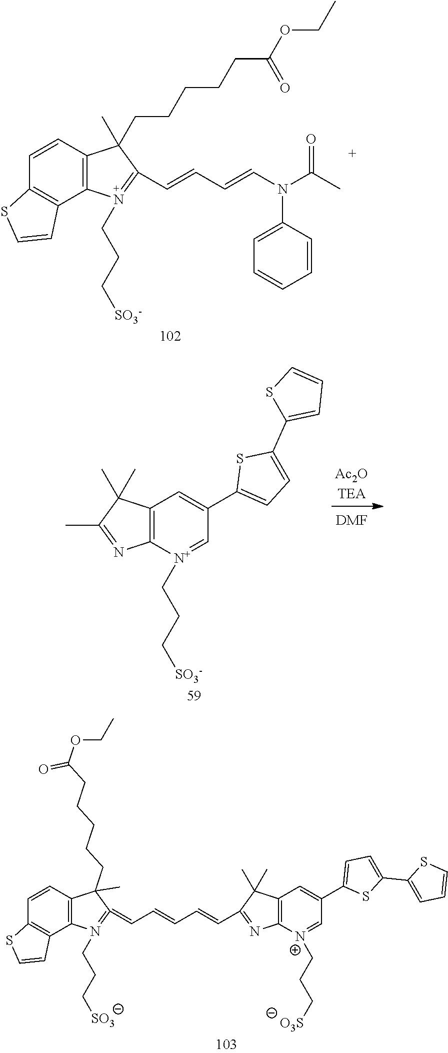

### Step 5: Synthesis of Compound 103

Compound 103 was prepared from Compound 102 and Compound 59 using the procedure described for the synthesis of Compound 49 in step 2 of Example 8. Excitation/Emission 723 nm/760 nm in water.

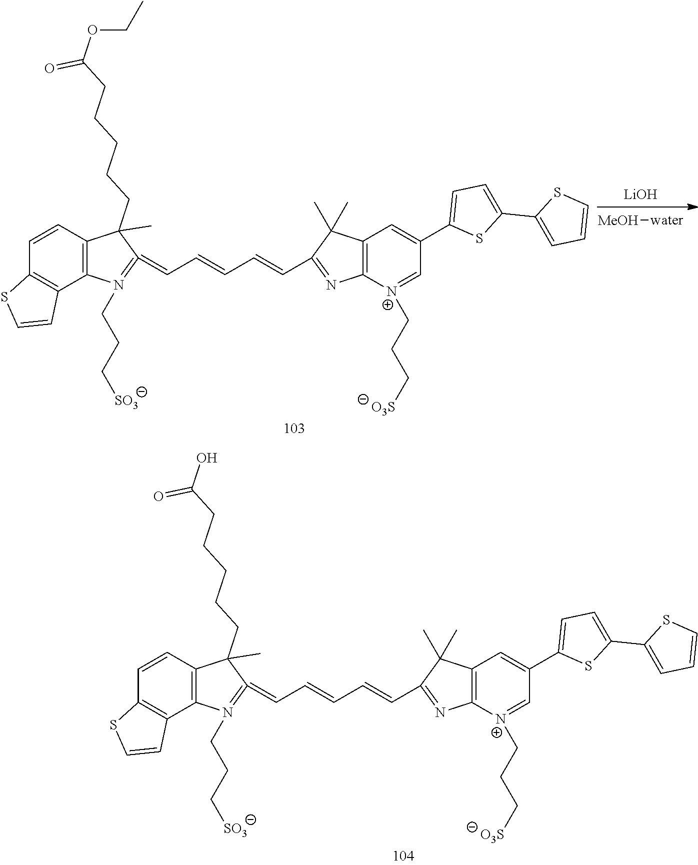

### Step 6: Synthesis of Compound 104

Compound 104 was prepared from Compound 103 using the procedure described for the synthesis of Compound 61 in step 4 of Example 10. Excitation/Emission 723 nm/760 nm in water.

### Example 20: Synthesis of Compound 110

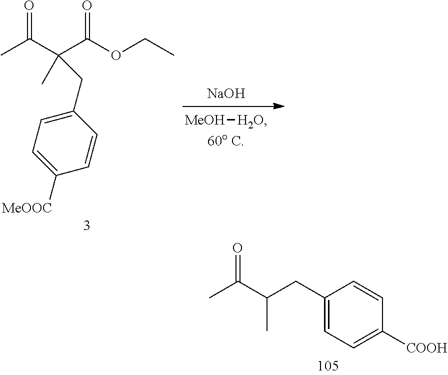

### Step 1: Synthesis of Compound 105

Compound 105 was prepared from Compound 3 using the procedure described for the synthesis of Compound 21 in step 1 of Example 2.

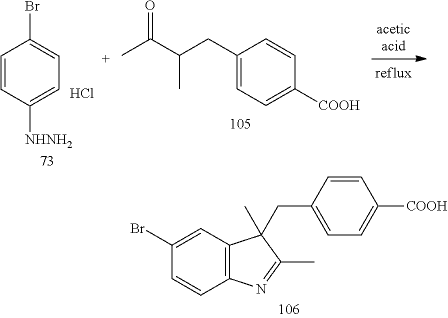

### Step 2: Synthesis of Compound 106

Compound 106 was prepared from Compound 73 and Compound 105 using the procedure described for the synthesis of Compound 74 in step 1 of Example 13.

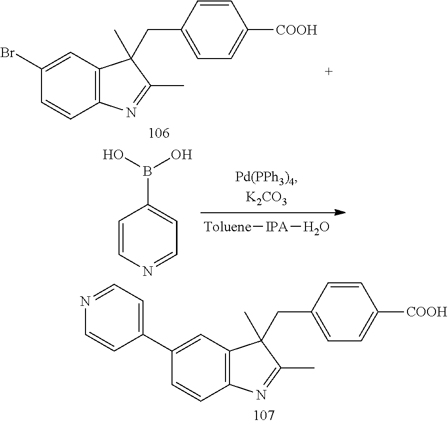

### Step 3: Synthesis of Compound 107

Compound 107 was prepared from Compound 106 and Compound 75 using the procedure described for the synthesis of Compound 76 in step 2 of Example 13.

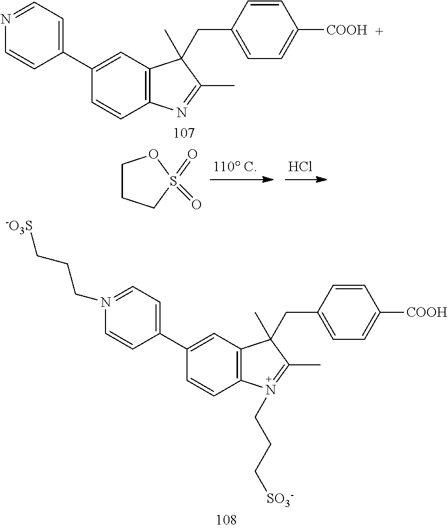

### Step 4: Synthesis of Compound 108

Compound 108 was prepared from Compound 107 using the procedure described for the synthesis of Compound 77 in step 3 of Example 13.

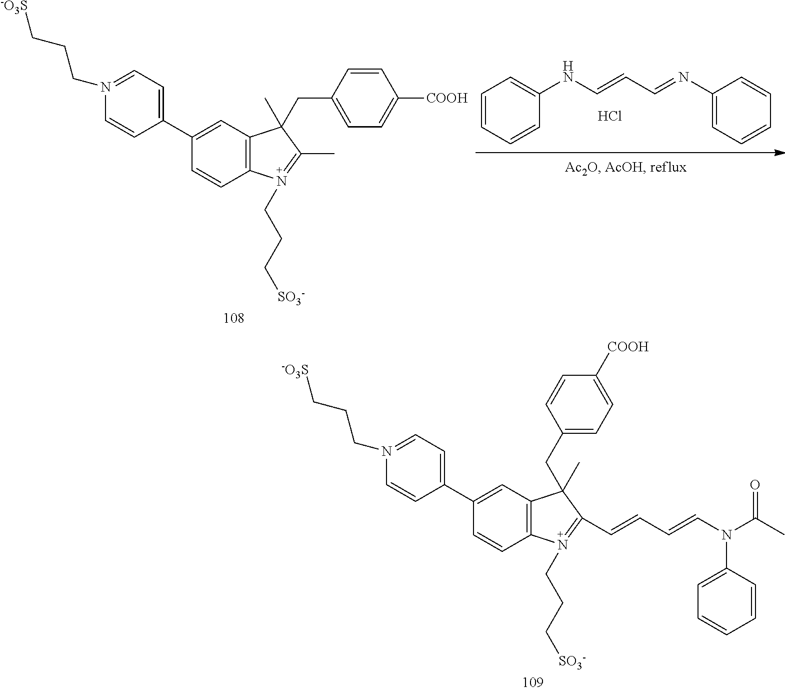

### Step 5: Synthesis of Compound 109

Compound 109 was prepared from Compound 108 using the procedure described for the synthesis of Compound 25 in step 4 of Example 2.

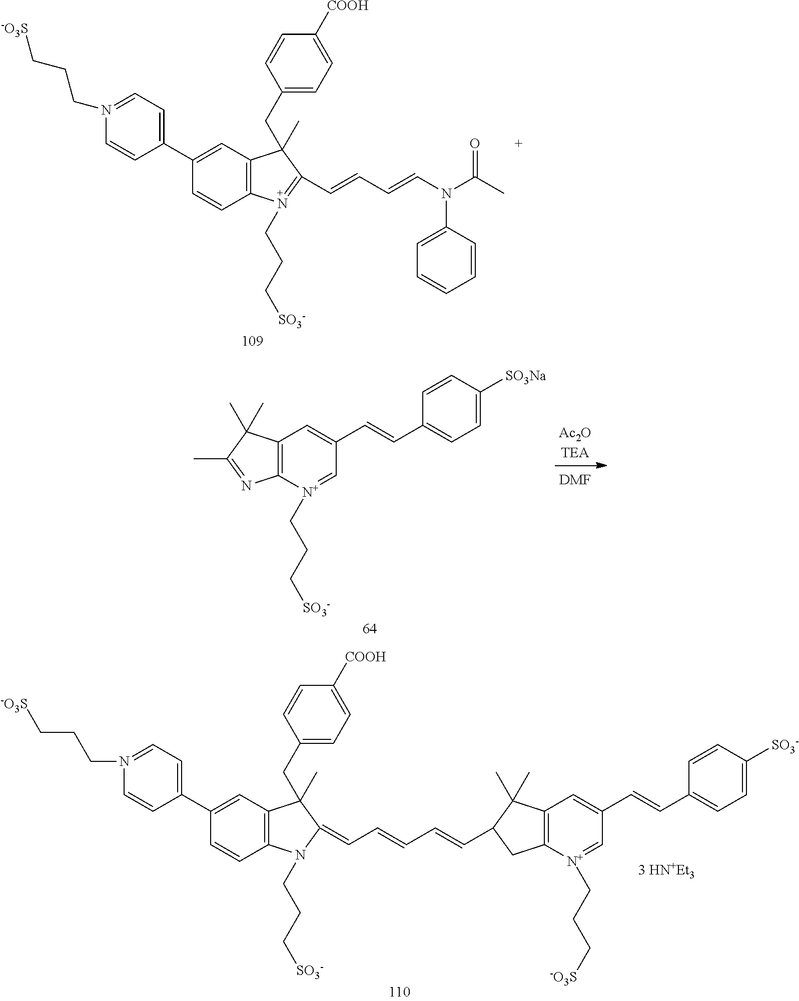

### Step 6: Synthesis of Compound 110

Compound 110 was prepared from Compound 109 and Compound 64 using the procedure described for the synthesis of Compound 60 in step 3 of Example 10. Excitation/Emission 708 nm/742 nm in water.

### Example 21: Synthesis of Compound 111

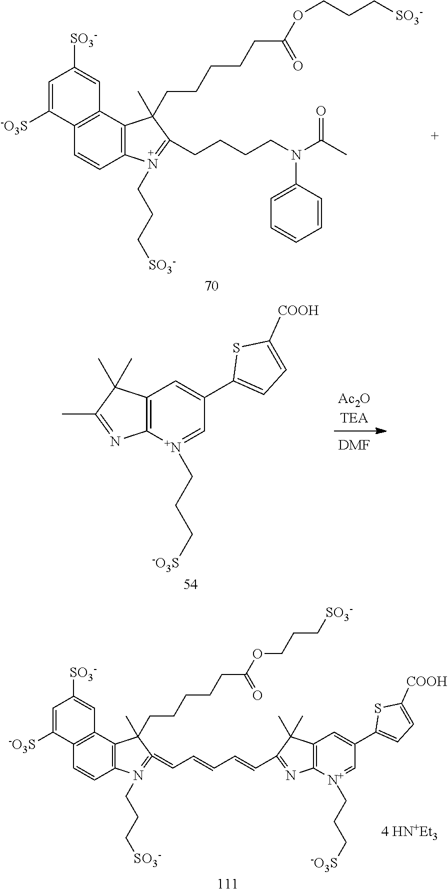

### Step 1: Synthesis of Compound 111

Compound 111 was prepared from Compound 70 and Compound 54 using the procedure described for the synthesis of Compound 71 in step 4 of Example 12. Excitation/Emission 705 nm/732 nm in water.

### Example 22: Quantum Yield Measurements

The absolute fluorescence quantum yields of representative compounds disclosed herein were measured with a Hamamatsu Quantum Yield Device (Hamamatsu C₁₀₀₂₇). The compounds were dissolved in water, and the absorbance at the absorption maxima for each compound was adjusted to 0.03-0.06 for QY determination. The quantum yield measurements for representative compounds disclosed herein in TEA salt form are shown in Table 2.

TABLE 2
Quantum Yield Measurements for Representative Azaindole Cyanine Compounds*

| | | | | Quantum |
|---|---|---|---|---|
| Compound | | Ex | Em | Yield |
| # | Structure (NHS ester) | (nm) | (nm) | (QY) |
| Comparator Compound A | 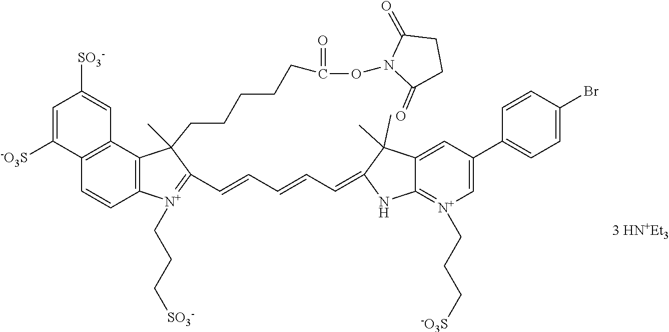 | 695 | 718 | 0.203 |
| 47 (Compound C) | 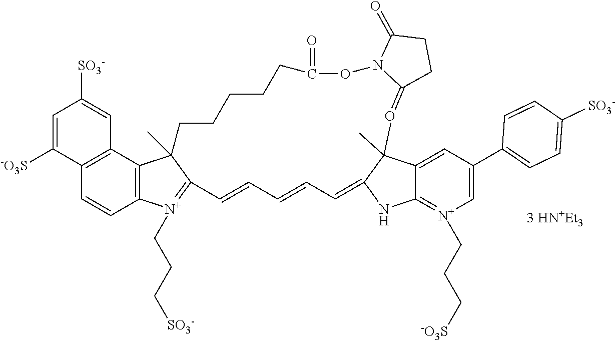 | 699 | 722 | 0.262 |
| 18 | 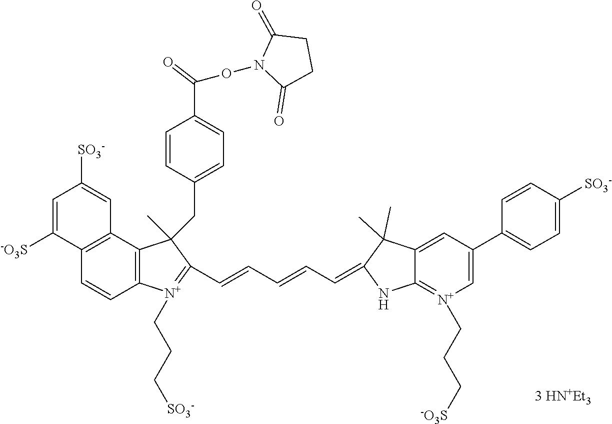 | 700 | 724 | 0.262 |
| 51 (Compound D) | 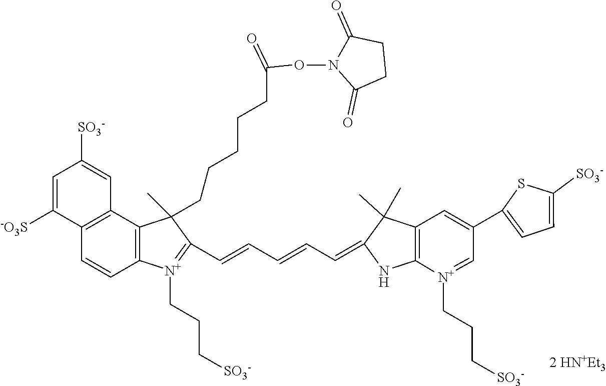 | 705 | 732 | 0.240 |
| 30 | 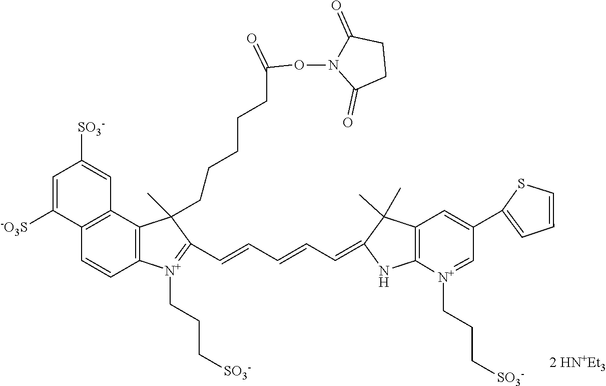 | 707 | 732 | 0.212 |
| 41 (Compound B) | 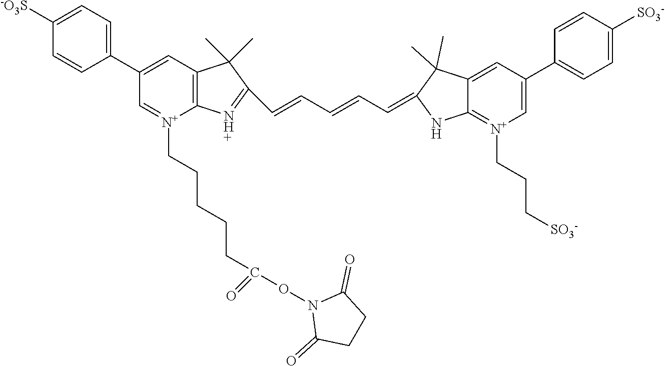 | 721 | 743 | 0.199 |
| 36 | 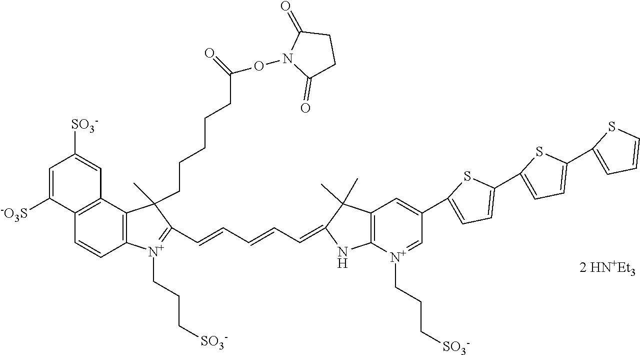 | 725 | 747 | 0.015 |

*Although counterions such as triethylamine are shown with certain compounds such as compounds 47, 18, 51, 30, and 36, reference to such compounds
does not include a particular counterion unless expressly indicated.

The quantum yield and emission properties for the tested compounds were particularly affected by varying the substitutions at the 5 position of the pyrrolopyridine ring. For example, replacement of the bromine substituent (bromo group) of Comparator Compound A with a sulfonated benzene group increases the quantum yield significantly. The quantum yields of 47 and 18 are about 1.3 times of that of Compound A; whereas substitution of the bromo group with a sulfonated thiophene group (51) shifts the absorption and emission maxima to the red (about 10 nm shift in absorption and 15 nm shift in emission) and increases the quantum yield by about 20% relative to Compound A. The introduction of sulfonated aromatic group on a pyrrolopyridine attached to the 5-membered polymethine bridge can shift the emission maximum to longer wavelengths in the far-red spectral region, while significantly improving quantum yield. The brightness of the cyanine compounds can be maintained even in the presence of additional sulfonate and alkyl sulfonate substituents introduced for the improvement of water solubility.

### Example 23: Compounds for Use in Flow Cytometry

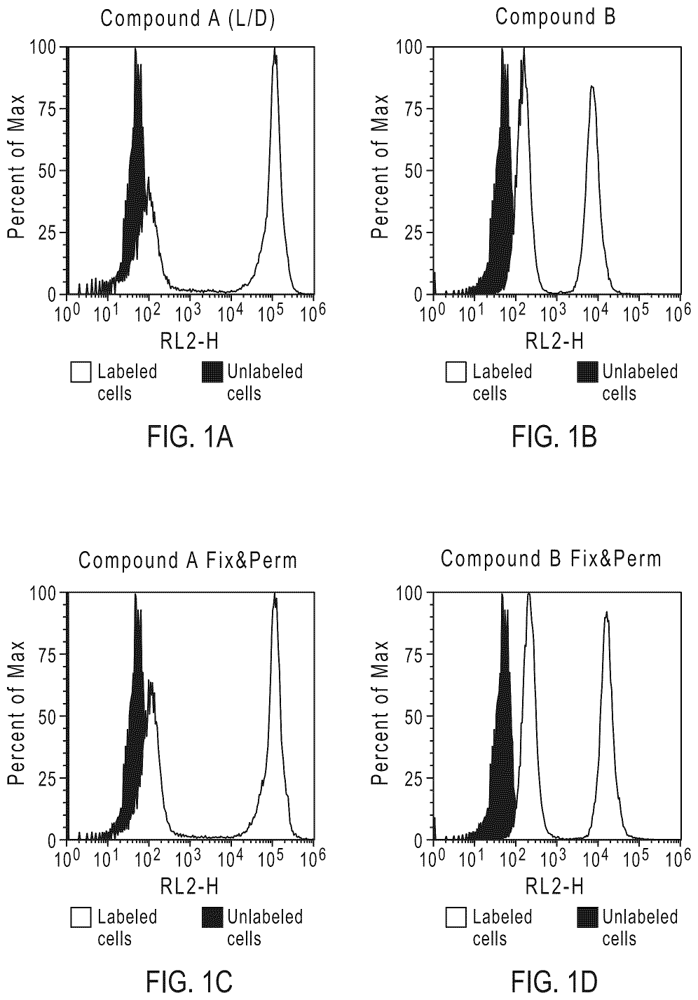

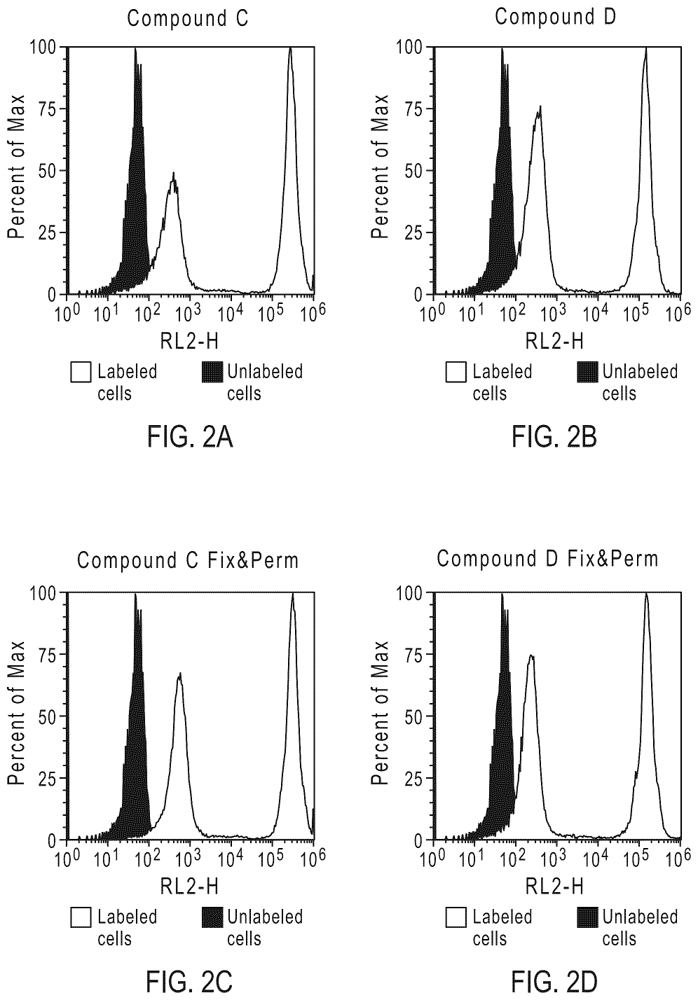

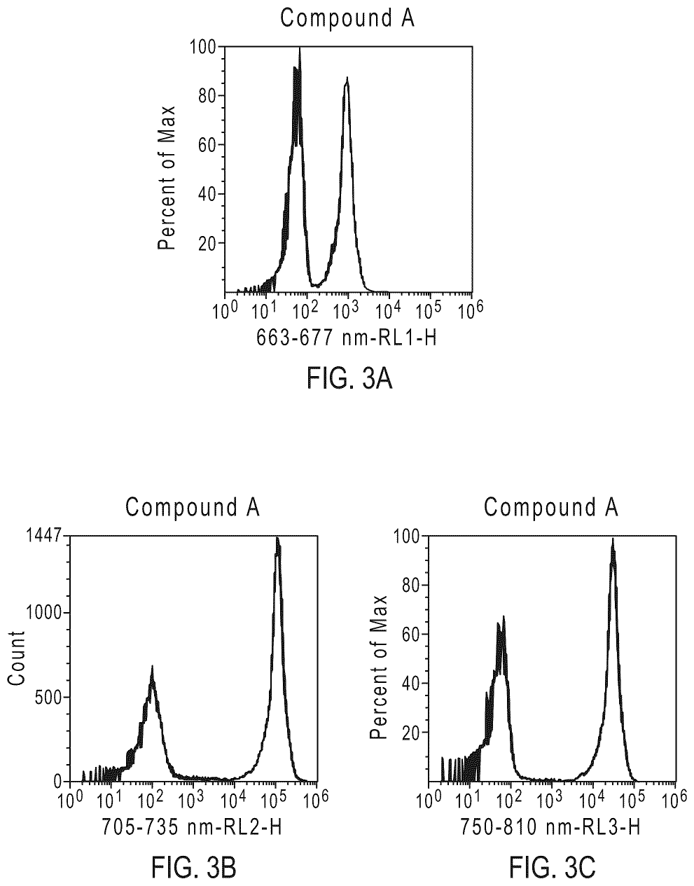

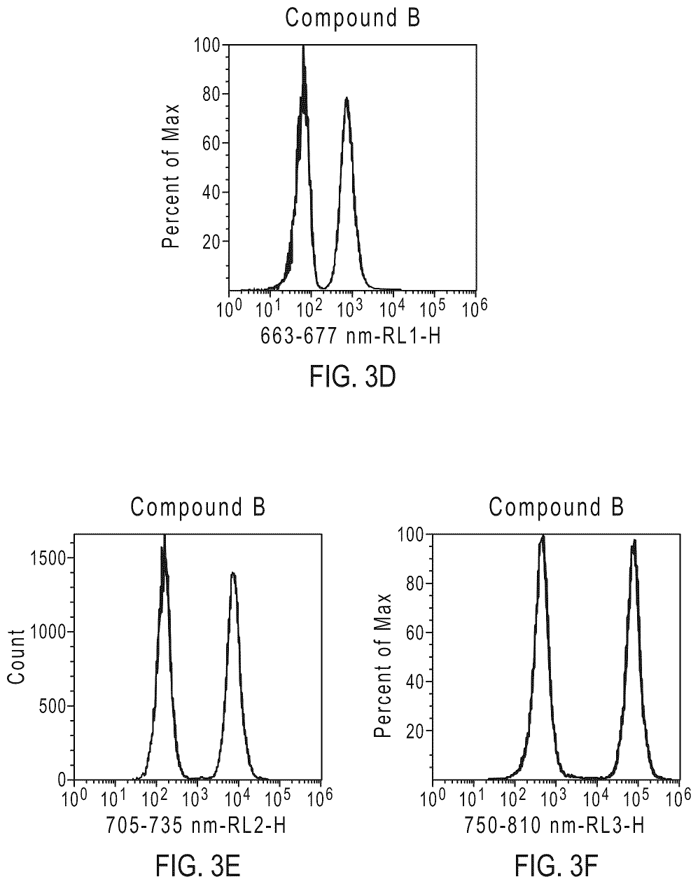

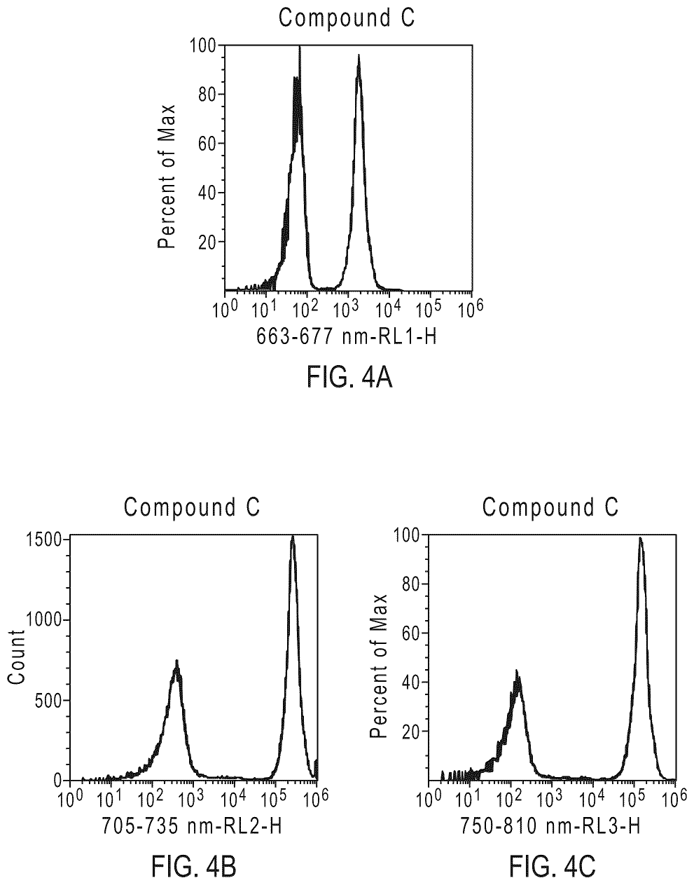

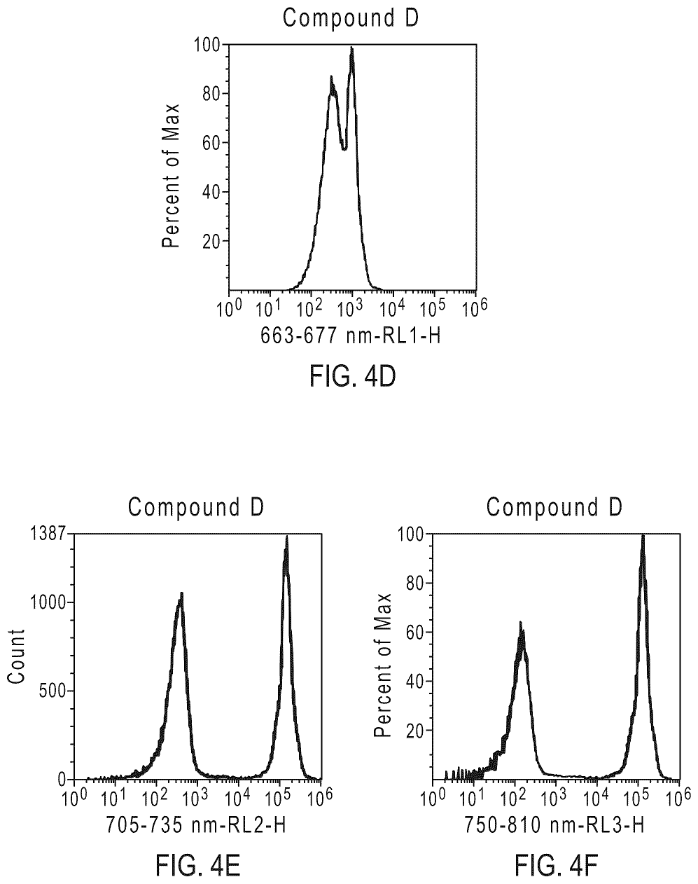

The following compounds were tested for use as dyes in flow cytometry: Comparator Compound A; Compound B (equivalent to 41); Compound C (equivalent to 47); and Compound D (equivalent to 51). A mixture of live and dead Jurkat cells were resuspended in PBS at 10⁶/mL. Dyes were dissolved in DMSO to 2 mM. 1 mL aliquots were labeled with each dye at 1 μM. Cells were incubated for 20 minutes at room temperature. Cells were washed and resuspended in 1 mL PBS. 50,000 events per sample were acquired with an Attune NxT Flow Cytometer (Thermo Fisher Scientific; Waltham, MA). Cells were centrifuged and resuspended in Invitrogen™ eBioscience™ IC Fixation Buffer for 15 minutes (available from Thermo Fisher Scientific), and the resuspended cells were centrifuged and resuspended in Invitrogen™ eBioscience™ IC Perm Buffer for 15 minutes (available from Thermo Fisher Scientific). The cells were analyzed again with the Attune NxT Flow Cytometer with PMT voltage set at 260 mV in all channels so brightness could be directly compared between channels. FIG. 1A-FIG. 1D and FIG. 2A-FIG. 2D show flow cytometry data for labeled and unlabeled cells. All four dyes can distinguish live and dead cells, even after labeled cells are fixed and permeabilized. FIG. 3A-FIG. 3F and FIG. 4A-FIG. 4F compare flow cytometry data for the four dyes across three emission channels with excitation using a 640 nm laser. The signal from Compound A is brightest in RL2 channel (705-735 nm), so it can be compensated out of the RL3 channel (750-810 nm), making it suitable for multiplexing with dyes emitting in the far-red region of the spectrum. All four dyes can be compensated out of the RL1 channel (663-677 nm), making them each suitable for multiplexing with dyes emitting in the red region of the spectrum, whereas Compounds B-D were brighter in RL3.

### Example 24: Spectral Cell Sorter Analysis

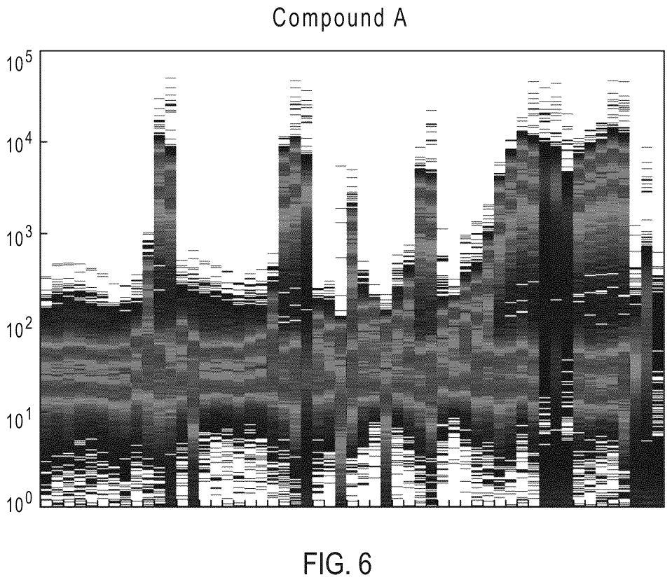

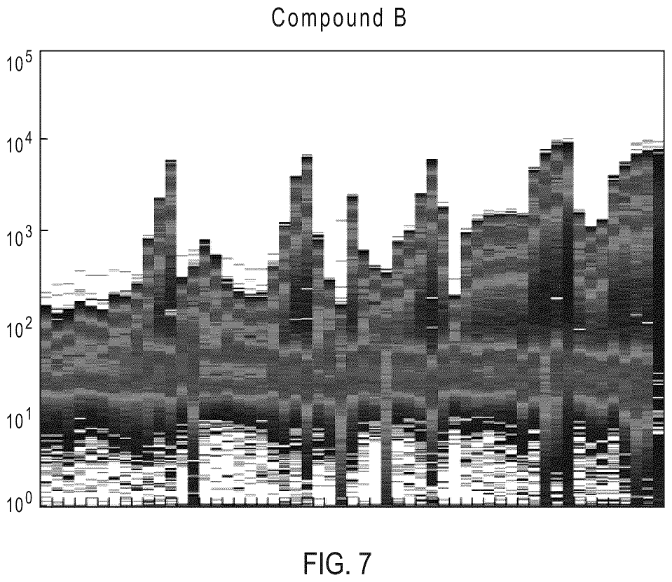

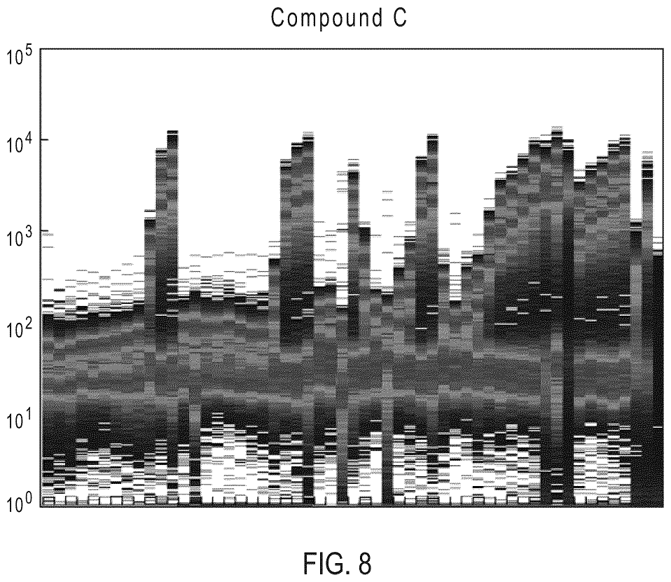

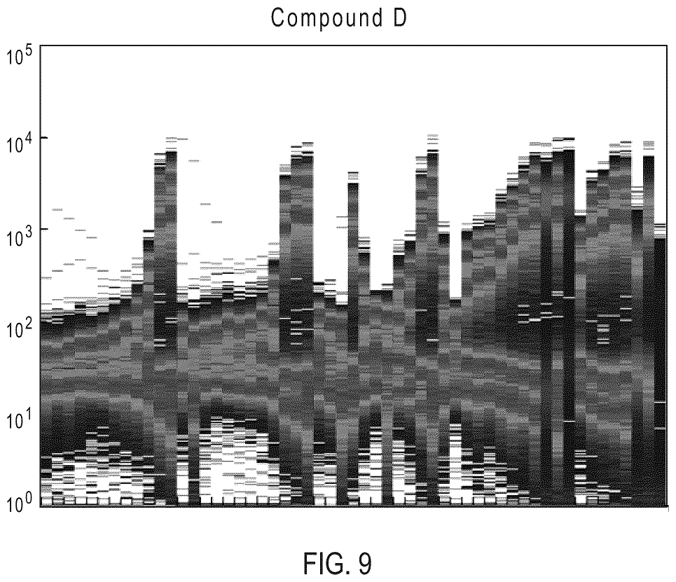

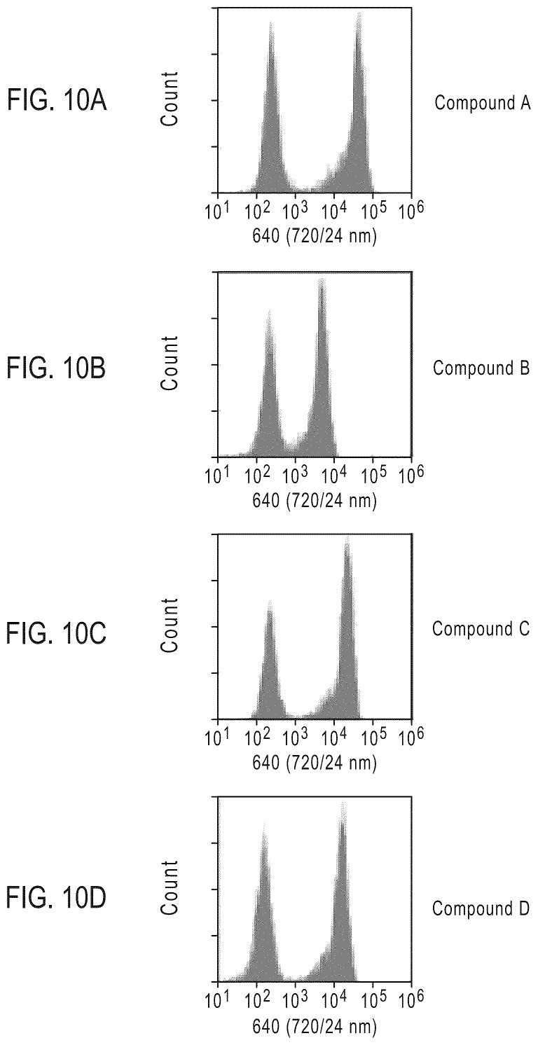

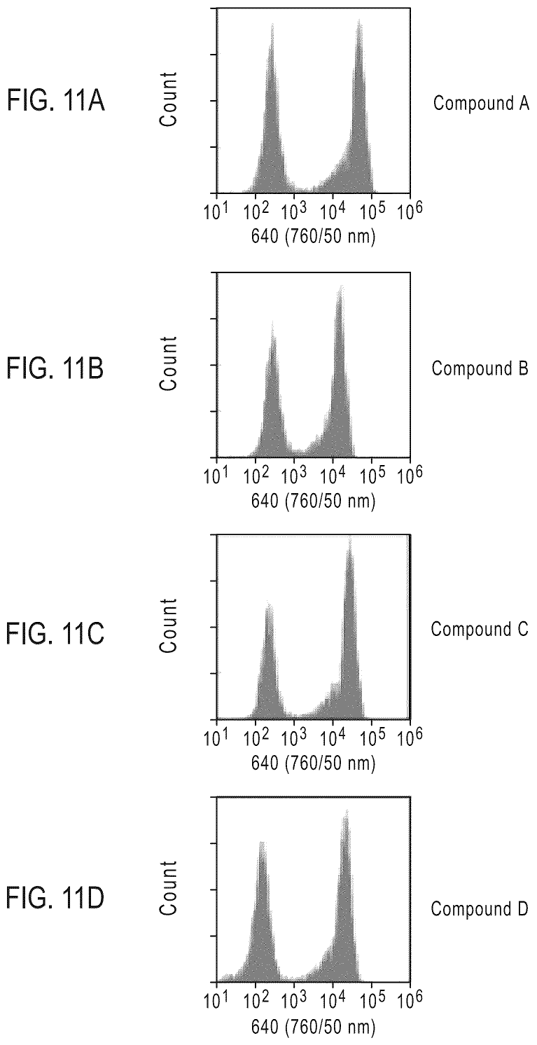

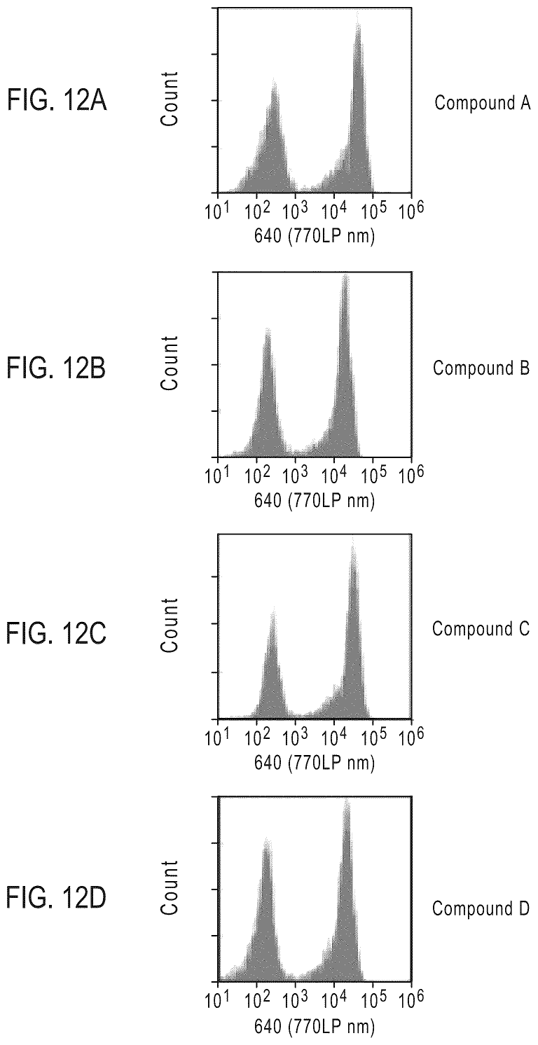

Cells labeled with Compounds A-D, as described in Example 23 were analyzed using a Bigfoot Spectral Cell Sorter (Thermo Fisher Scientific). This data was analyzed with Sasquatch Software (Thermo Fisher Scientific) and is shown in FIG. 6-FIG. 9. 10,000 cells per sample were analyzed with a Bigfoot Spectral Cell Sorter with 7 lasers (349 nm; 445 nm; 405 nm; 488 nm; 561 nm; 640 nm; 785 nm). FIG. 6-FIG. 8 show spectral signal graphs for live and dead cells labeled with compounds A-D. FIG. 6 is a spectral graph for Comparator Compound A. FIG. 7 spectral graph for compound B. FIG. 8 is a spectral graph for compound C. FIG. 9 is a spectral graph for compound D. FIG. 10A-FIG. 12D show flow cytometry data to compare the four dyes across three emission channels with excitation using a 640 nm laser. Each of the four compounds A-D can clearly distinguish live and dead cells while also demonstrating slightly different spectral profiles in the three emission channels shown.

### Example 25: Photostability Study of the Dyes

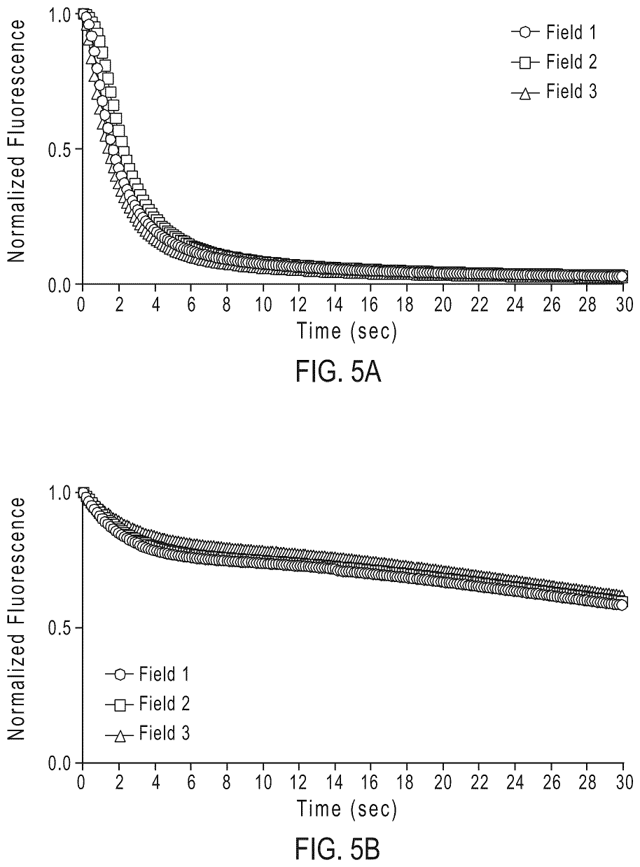

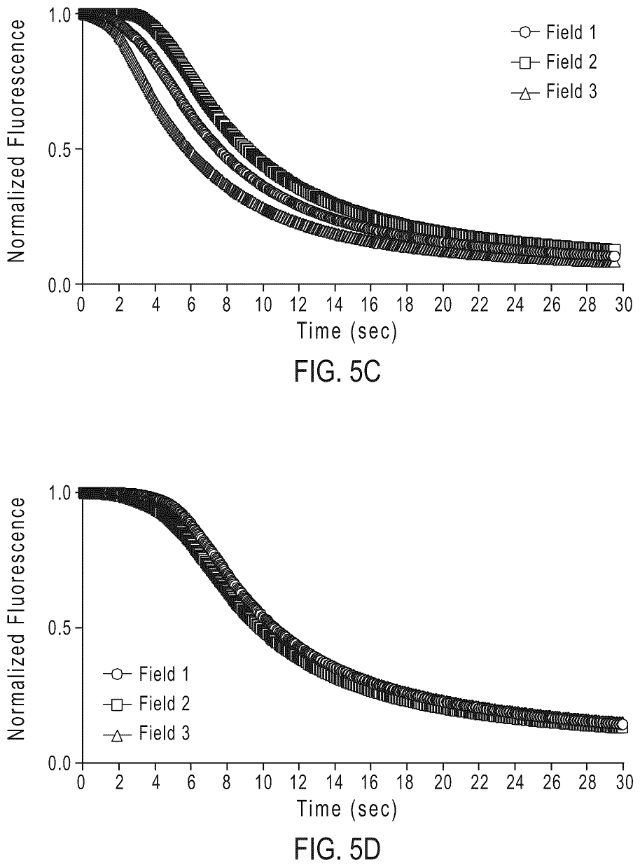

U20S cells were fixed (4% formaldehyde in DPBS for 15 min) and permeabilized (0.5% Triton X-100 in DPBS for 15 min). Cells were stained with 2 μM dye in DPBS for 30 min at RT, washed 3× and imaged in DPBS. Stained cells were imaged on an EVOS M7000 Imaging System with 20× objective and CY5.5 light cube (Thermo Fisher Scientific). The dyes were irradiated with the same light intensity, gain and duration (30 seconds). The normalized fluorescence was measured over time for the following dyes and three different fields of view. Photobleaching curves are shown for Comparator Compound A (FIG. 5A), Compound 41 (FIG. 5B), Compound 51 (FIG. 5C), and Compound 47 (FIG. 5D).

### Example 26: Thermal Stability Evaluation

Representative NHS ester compounds, as well as Alexa Fluor 647 (AF647) and Alexa Fluor 676 (AF676) NHS ester compounds from Thermo Fisher Scientific, were each coupled to an identical oligonucleotide (“oligo”). This was added into a PCR master mix and then subjected to 60 cycles of PCR (thermal) cycling using Applied Biosystems Verity 96 Well thermal cycler. Samples were then analyzed by fluorescence using a Jasco FP-8600 spectrofluorometer and by UPLC using a Vanquish UHPLC System from Thermo Fisher Scientific and compared against compounds prior to enduring the PCR thermal cycling procedure (see Table 3). The tested compounds were thermally stable under rigorous PCR cycling with minimal change relative to initial fluorescence intensity or percent change by UPLC.

TABLE 3
PCR Thermal Stability Results for Representative Compounds

| | Change in | |
|---|---|---|
| | Fluorescence | Change in UPLC |
| Compound # | Intensity (%) | Signal Intensity |
| AF647-oligo | 1.90 | not measured |
| AF676-oligo | 2.90 | not measured |
| 47-oligo | −3.90 | −6.30% |
| 30-oligo | −8.50 | −5.20% |
| 41-oligo | −1.00 | not measured |

### Example 27: Scientific Analysis Systems, Computer-Implemented Methods, Computing Devices, and Computer-Readable Media

The present disclosure also relates to scientific analysis systems, as well as related computer-implemented methods, computing devices, and computer-readable media, that utilize data generated by processes that involve the novel compounds disclosed herein.

For example, the systems, computer-implemented methods, computing devices, and computer-readable media disclosed herein may utilize data generated as part of imaging applications involving any of the novel compounds disclosed herein, such as immunohistochemistry, FISH, cell tracing, receptor labeling, and cytochemistry. In another example, these systems, computer-implemented methods, computing devices, and computer-readable media may utilize data generated by use of the novel compounds disclosed herein for probing biological structure, function, and interactions.

In some embodiments, the systems, computer-implemented methods, computing devices, and computer-readable media disclosed herein may be configured to detect at least one biomolecule based on data indicative of the combination of the at least one biomolecule with a composition comprising at least one excipient and a compound of Formula (I) or (II) in an effective concentration to label the at least one biomolecule under conditions sufficient for binding the compound to the biomolecule.

In some embodiments, the systems, computer-implemented methods, computing devices, and computer-readable media disclosed herein may utilize data generated by an assay selected from fluorescence microscopy, flow cytometry, in vivo imaging, immunoassay, hybridization, chromatographic assay, electrophoretic assay, microwell plate based assay, FRET system, BRET system, high throughput screening, or microarray. In another example, the data may be generated by in vivo imaging comprising providing the biomolecule-bound compound to at least one of a biological sample, tissue, or organism, and detecting the biomolecule within the at least one of a biological sample, tissue, or organism.

In some embodiments, the systems, computer-implemented methods, computing devices, and computer-readable media disclosed herein may be configured to use data to detect or quantify biological material using fluorescent dyes including the novel compounds disclosed herein. For example, the data may be generated by optical techniques, including fluorescence optical, qualitative and/or quantitative determination methods to diagnose properties of cells (molecular imaging), in biosensors (point of care measurements), for investigation of the genome, or in miniaturizing technologies. In another example, the data may be generated by microscopy, super-resolution imaging, cytometry, cell sorting, FCS, uHTS, me-FISH, FRET-systems, BRET-systems, or microarrays (DNA- and protein-chips). In some embodiments, the systems, computer-implemented methods, computing devices, and computer-readable media disclosed herein may be configured to use data indicative of fluorescence emission to perform any detection or quantitation techniques involving the novel compounds disclosed herein.

In some embodiments, the systems, computer-implemented methods, computing devices, and computer-readable media disclosed herein may be configured to use data to detect or quantify antigens using the novel compounds disclosed herein. For example, the data may be generated by immunofluorescence, immunohistochemistry, flow cytometry (e.g., FACS), Western blotting, fluorescence ELISA, and any other approach where fluorescently labeled antibodies can be used.

In some embodiments, the systems, computer-implemented methods, computing devices, and computer-readable media disclosed herein may be configured to use data (e.g., fluorescent signals) to assess cell viability generated by techniques utilizing the novel compounds disclosed herein. For example, the systems, computer-implemented methods, computing devices, and computer-readable media disclosed herein may use data to quantifying the amount of live and/or dead cells, e.g., by determining whether the degree of fluorescence exceeds a predetermined threshold (indicating nonviability), or by grouping results into live and dead populations (where dead cell populations show a greater extent of staining given their compromised membrane integrity).

In some embodiments, the systems, computer-implemented methods, computing devices, and computer-readable media disclosed herein may be configured to exclude dead cells from data generated by techniques utilizing the novel compounds disclosed herein, allowing cleaner separation and identification of cell populations.

In some embodiments, the systems, computer-implemented methods, computing devices, and computer-readable media disclosed herein may be configured to collect and analyze fluorescence data generated by techniques utilizing the novel compounds disclosed herein. In some embodiments, the data may be generated by a plate reader, fluorescence microscope, or flow cytometer. In certain embodiments, the fluorescence detection can be achieved using an acoustic focusing flow cytometer, such as the ATTUNE NxT Flow Cytometer from Thermo Fisher Scientific. In other embodiments, fluorescence detection can be achieved using a cell sorting flow cytometry instrument, such as the Invitrogen Bigfoot Spectral Cell Sorter from Thermo Fisher Scientific.

In some embodiments, the systems, computer-implemented methods, computing devices, and computer-readable media disclosed herein may be configured to spectrally distinguish multiple fluorescent molecules, as disclosed herein.

In some embodiments, the systems, computer-implemented methods, computing devices, and computer-readable media disclosed herein may be configured to collect or analyze data generated by techniques involving the novel compounds disclosed herein and prolonged heat exposure and/or extended irradiation, such as PCR and fluorescence imaging.

In some embodiments, the systems, computer-implemented methods, computing devices, and computer-readable media disclosed herein may be configured to collect or analyze data generated by techniques involving the novel compounds disclosed herein, such as multiplex flow cytometry experiments where bright and stable fluorescent dyes in this region of the spectrum are needed, multicolor panels in both conventional and spectral flow cytometry applications, or multi-color imaging experiments where bright and stable fluorescent dyes in this region of the spectrum are needed.

In some embodiments, the systems, computer-implemented methods, computing devices, and computer-readable media disclosed herein may be configured to collect or analyze data generated by techniques involving the novel compounds disclosed herein, such as labeling or detecting nucleic acids in a wide variety of samples, such as in aqueous solutions, sequencing or amplification reactions such as PCR, cellular samples (e.g., for FISH or general nuclear or chromosome staining) and electrophoretic gels.

In some embodiments, the systems, computer-implemented methods, computing devices, and computer-readable media disclosed herein may be configured to collect or analyze data generated by techniques involving the novel compounds disclosed herein, with examples of such data including characteristics of a compound-nucleic acid complex, including the presence, location, intensity, excitation and emission spectra, fluorescence polarization, fluorescence lifetime, and other physical properties of the fluorescent signal. In some embodiments, the systems, computer-implemented methods, computing devices, and computer-readable media disclosed herein may be configured to collect or analyze data generated by techniques involving the novel compounds disclosed herein to detect, differentiate, sort, quantitate, and/or analyze aspects or portions of the sample.

Any of the computational methods discussed herein may include any known data collection, storage, analysis, and decision-making techniques. For example, any suitable ones of the computational methods discussed herein may include machine-learning or other analytical techniques to process and understand the data generated by scientific experiments and other processes involving the novel compounds disclosed herein. Such techniques may include supervised learning (i.e., training a machine learning model using labeled data to predict or classify new observations), unsupervised learning (i.e., identifying patterns and structures within data without pre-existing labels using techniques such as clustering), dimensionality reduction (i.e., techniques for simplifying or filtering a complex data set, such as principal component analysis (PCA)), or deep learning (i.e., utilizing neural network computational methods with multiple layers to extract intricate patterns and representations from data), statistical analysis (e.g., hypothesis testing, regression analysis, analysis of variance (ANOVA), and correlation analysis.

In some embodiments, any suitable ones of the computational methods disclosed herein may include analyzing data, generated by imaging (e.g., fluorescence microscopy), using machine-learning, as known in the art. Examples of such embodiments may include using machine-learning to perform object detection and segmentation in imaging data (e.g., to identify cells or subcellular structures), image classification (e.g., to classify different types of structures or identify specific cellular events based on patterns in fluorescence images), or performing image quality and denoising.

In some embodiments, any suitable ones of the computational methods disclosed herein may include analyzing data, generated by flow cytometry, using machine-learning, as known in the art. Examples of such embodiments may include using machine-learning to perform cell population identification and classification (e.g., by identifying and classifying different cell types) or to perform correction for artifacts (e.g., spectral overlap or fluorescence spillover).

Also, provided herein are scientific instruments that can detect a compound, as disclosed herein. In certain embodiments, the compound is bound to a biomolecule or biological material, as disclosed herein. In certain embodiments, the scientific instrument can be used to generate a detectable response from the compound and can further include a computational device for analysis of the detectable response. Detection of the detectable response by the computational device can further include performing a machine-learning analysis technique on a data set of detectable responses generated by the instrument. The machine-learning technique can use a computer process to generate a machine learning model, and the machine learning model based on the model data and a prediction process can be trained to predict and/or classify new observations.

Thus, in one aspect a method is provided that includes determining an attribute, by a computing device, as a function of a machine-learning process, wherein data generated from one or more dyes, as disclosed herein, and collected with an analyzer electronically connected to the computing device is inputted into the machine-learning process. An analyzer (e.g., a scientific instrument) that is electronically connected to a computing device is used to generate data from a compound, as disclosed herein. The data serves as input for a machine-learning process that is conducted by the computing device, where the attribute can be determined by the computing device as a function of the machine learning process. In some embodiments, the output of the machine-learning process provides information about the attribute. It is contemplated that the described method can be used by any type of analyzer, including, but not limited to, a fluorescence microscope, flow cytometer, cell sorter, mass spectrometer, mass cytometer, imaging cytometer, fluorescence resonance energy transfer (FRET) system, bioluminescence resonance energy transfer (BRET) system, and an electron microscope. In some embodiments, the analyzer is configured to collect data for an assay selected from the group consisting of an in vivo imaging assay, an immunoassay, a hybridization assay, a, chromatographic assay, an electrophoretic assay, a microwell plate-based assay, a high throughput screening assay, and a microarray-based assay; or to collect data gene.

In another aspect, a machine-learning process can be included in the method for configuring the analyzer prior to collection of the data generated from the one or more dyes. The analyzer can be configured with a set of configuration settings, where the configuration settings can be determined based on the type of sample to be interrogated or the desired type of analysis or experiment. The configuration settings can be adjusted manually or can be automated. In certain embodiments, the configuration settings can be determined using a machine learning process that is distinct from machine learning process used to determine an attribute based on the data generated from the dye. An exemplary method can include processing data from an analyzer using a machine-learning process to adjust or predict configuration issues to adjust configuration settings associated with the analyzer. The method can configure settings based on the type of sample to be interrogated or to aid in selection of an appropriate experiment by the researcher. Representative examples of configuration settings include, e.g., voltage (e.g, plate voltage, photomultiplier tube voltage, or electrical charging ring voltage), area selection, flow rate, filter position, gates, alignment and calibration settings, wavelength or laser selection, and the like. In a specific example, the analyzer can be a flow cytometer or cell counting system, and the configuration settings can include, e.g., voltage (e.g., plate voltage, photomultiplier tube voltage, or electrical charging ring voltage) or an area corresponding to gating of cells.

The attribute that can be determined using methods disclosed herein can be any property or characteristic relating to the sample that is being interrogated by the analyzer, such as a structure, a function, or an interaction. For example, the attribute can be a physical or chemical characteristic of a structure, such as a molecule, particle, or a biological material (e.g., a cell or issue). The attribute of the structure can be, for example, size, shape, complexity, concentration, chemical composition, and the like. In certain embodiments, the attribute can be an interaction, such as the interaction between the compound (or a substance to which the compound is hound) and a structure. For example, the attribute can be information about the type and/or number of structures (e.g., cells or molecules) to which the compound is bound. The interaction can be between the compound (or a substance to which the compound is bound) and another molecule. By way of a specific example, a dye compound, as disclosed herein, can be hound to a protein (e.g., an antibody) that can recognize a protein on the surface of or within the interior of a biological cell. Upon interrogation by an analyzer, such as a fluorescence microscope or flow cytometer, the analyzer detects an emission signal from the dye compound. The data can be input into a computing device that is electronically connected to the analyzer. The computing device can conduct a machine-learning process on the emission data from the compound, whereby the machine learning process determines an attribute, such as the identity or concentration of a cell population. In other embodiments, the attribute can be a function, such as cellular function, including without limitation, protein expression patterns and gene expression, live and dead cell distinction, or cell cycle stage.

In certain embodiments, the attribute is a spectral property, a measure of spectral overlap or an artifact in the data. In certain embodiments, the machine learning process corrects artifacts in the data, removes noise, or normalizes the data, such that the data is in a suitable format for subsequent analysis. Thus, certain methods include processing the data using the computing device to remove noise, correct for artifacts, and/or to normalize the data, such that the data is in a suitable format for subsequent analysis. Certain methods further include one or more of the following steps: extracting one or more features from the processed data to capture detectable responses of interest; training the machine learning process from the extracted features, such that the process learns patterns or relationships between the extracted features; validating the trained machine learning process to assess the performance and ability of the process to accurately classify or predict patterns or relationships based on the detectable responses; or applying the trained process to a predict or classify detectable responses by making predictions based on the trained patterns or relationships. In certain embodiments, methods disclosed herein can further include training, by the computing device, the machine-learning process, using training data correlating the data from the one or more fluorescent dyes to the attribute.

In yet another aspect, provided herein is one or more non-transitory computer-readable media having instructions thereon that, when executed by one or more processing devices of one or more computing devices, cause the one or more computing devices to perform the method disclosed herein. Also provided herein is a computing device configured to perform the method described herein is provided.

In yet another aspect, an apparatus is provided that includes a computing device, wherein the computing device comprises: at least one processor comprising memory, wherein the memory includes instructions to: determine an attribute as a function of a machine-learning process, wherein data generated from one or more dyes, as described herein, and collected from an analyzer electronically connected to the computing device is inputted into the machine-learning process,
In any of the methods involving a machine-learning process or apparatus for performing a machine-learning process, one or more dyes, as disclosed herein, can be bound to a biomolecule, such as, for example, a protein, polypeptide, antibody, enzyme, nucleic acid, nucleoside triphosphate, oligonucleotide, biotin, hapten, cofactor, lectin, antibody binding protein, carotenoid, carbohydrate, hormone, neurotransmitter, growth factors, toxin, biological cell, lipid, receptor binding drug, fluorescent proteins, or a combination of biomolecules. In any of the methods involving a machine-learning process, the dye can emit light in the far-red and/or near-IR region of the electromagnetic spectrum upon excitation at an appropriate wavelength of light. In some cases, the dye can have an excitation profile in the far-red region of the electromagnetic spectrum. In other cases, the dye has an excitation profile in the near-IR region of the electromagnetic spectrum. The dye can be a cyanine compound, e.g., an azaindole cyanine compound, as described herein. In certain embodiments, the dye is a compound of Table A,
Methods described herein can be implemented with any number of scientific instruments adapted for detecting a response from a compound, as disclosed herein. By way of illustration, one representative method implements a flow cytometer to detect one or more optical signals generated by a sample. The sample can include one or more compounds, as disclosed herein. In certain embodiments, the compound is bound to or incorporated into a cell. In certain embodiments, the method can be used to analyze a sample that includes a plurality of eells, where at least one cell in the sample is bound to the compound. A computational device can be associated with the flow cytometer to analyze data generated by the instrument, Machine learning techniques can be particularly useful for processing large and complex datasets produced from samples that include a multitude of different detectable responses. For examrnple, a sample can include a compound, as disclosed herein, and can further include one or more other types of detectable labels to provide a sample that produces multiple types of detectable responses when analyzed using a scientific instrument. In certain embodiments, the collected data includes a series of detectable responses from the one or more detectable labels (e.g., dyes). For example, the detectable response can be fluorescence emission and/or light scattering.

When methods are used to process data generated by a flow cytometer, such methods can leverage machine-learning for various purposes. Machine learning processes can be used to generate training sets that leverage a host of data produced by the flow cytometer. In one example, machine learning is used to remove noise, correct artifacts, and normalize data. In another example, machine learning can aid in clustering and cell population identification. Machine learning processes can be applied to identify distinct cell populations within a flow cytometry data set by grouping cells based on similarity in marker expression patterns, thereby allowing for identification or rare or abnormal cell populations. In another example, machine learning can be used to help visualize data more effectively by reducing dimensionality resulting from high dimensional datasets that contain many markers. In yet another example, machine learning can be used for classification and prediction, e.g., to classify cells in categories based on marker expression patterns that distinguish between healthy and diseased cells.

In a representative computational method, a flow cytometer is provided with a computational device that detects responses from a compound, as disclosed herein, such as fluorescence intensity or scatter properties, to generate a data set that can be processed with a machine learning process. Detectable responses measured by the flow cytometer can be processed and then used as input data for training machine learning process, where the processes can learn from the detectable responses to classify or predict specific outcomes.

In an exemplary flow cytometry workflow using a machine learning process, the flow cytometer measures fluorescence intensity and/or scatter properties of cells labeled with a compound, such as disclosed herein, to generate a series of measurements that can represent the response of various markers or characteristics for each cell. The raw data is processed to remove any noise, correct for artifacts, and to normalize the data, such that the data is in a suitable format for subsequent analysis. Relevant features then are extracted from the processed data to capture detectable responses of interest, e.g., fluorescence intensities of specific markers and/or scatter properties). The extracted features can be used to train the machine learning process, such that the process can learn patterns or relationships between the features (e.g., to distinguish specific cell types or disease states). The trained machine learning process can be evaluated and validated to assess its performance and its ability to accurately classify or predict based on the detectable responses, Once trained and validated, the process can be applied to new data to predict or classify cells based on their detectable responses by making predictions based on the learned patterns or relationships.

The computational methods disclosed herein may be implemented or facilitated in any of a number of embodiments. For example, the embodiments disclosed herein include one or more non-transitory computer-readable media having instructions thereon that, when executed by one or more processing devices of one or more computing devices, cause the one or more computing devices to perform any of the computational methods disclosed herein.

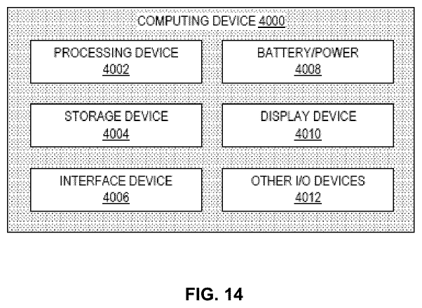

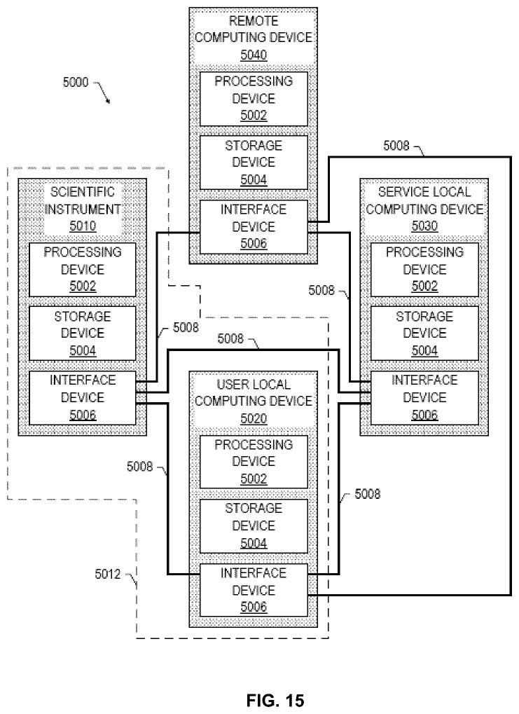

The computational methods disclosed herein may include interactions with a human user (e.g., via the user local computing device 5020 discussed herein with reference to FIG. 15). These interactions may include providing information, representative of an experiment or other process including any of the novel compounds disclosed herein, to the user (e.g., information regarding the operation of a scientific instrument such as the scientific instrument 5010 of FIG. 15, information regarding a sample being analyzed or other test or measurement performed by a scientific instrument, information retrieved from a local or remote database, or other information) or providing an option for a user to input commands (e.g., to control the operation of a scientific instrument such as the scientific instrument 5010 of FIG. 15, or to control the analysis of data generated by a scientific instrument), queries (e.g., to a local or remote database), or other information. In some embodiments, these interactions may be performed through a graphical user interface (GUI) that includes a visual display on a display device (e.g., the display device 4010 discussed herein with reference to FIG. 14) that provides outputs to the user and/or prompts the user to provide inputs (e.g., via one or more input devices, such as a keyboard, mouse, trackpad, or touchscreen, included in the other I/O devices 4012 discussed herein with reference to FIG. 14). The scientific instrument support systems disclosed herein may include any suitable GUIs for interaction with a user.

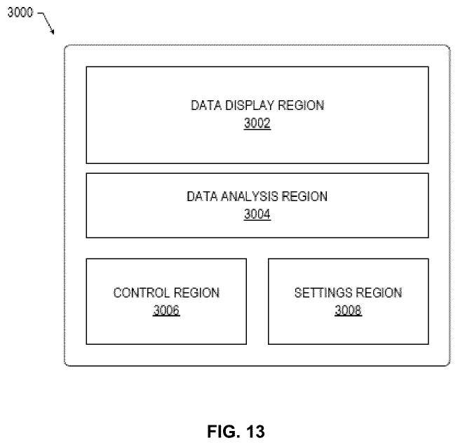

FIG. 13 depicts an example GUI 3000 that may be used in the performance of some or all of the computational methods disclosed herein, in accordance with various embodiments. As noted above, the GUI 3000 may be provided on a display device (e.g., the display device 4010 discussed herein with reference to FIG. 14) of a computing device (e.g., the computing device 4000 discussed herein with reference to FIG. 14) of a system (e.g., the scientific instrument support system 5000 discussed herein with reference to FIG. 15), and a user may interact with the GUI 3000 using any suitable input device (e.g., any of the input devices included in the other I/O devices 4012 discussed herein with reference to FIG. 14) and input technique (e.g., movement of a cursor, motion capture, facial recognition, gesture detection, voice recognition, actuation of buttons, etc.).

The GUI 3000 may include a data display region 3002, a data analysis region 3004, a scientific instrument control region 3006, and a settings region 3008. The particular number and arrangement of regions depicted in FIG. 13 is simply illustrative, and any number and arrangement of regions, including any desired features, may be included in a GUI 3000. Data representations like those of FIGS. 1-12 may be included in any appropriate region of a GUI (e.g., in the data display region 3002 or the data analysis region 3004 of the GUI 3000)).

The data display region 3002 may display data generated by a scientific instrument (e.g., the scientific instrument 5010 discussed herein with reference to FIG. 15). For example, the data display region 3002 may display data indicative of an experiment or other process involving any of the novel compounds disclosed herein.

The data analysis region 3004 may display the results of data analysis (e.g., the results of analyzing the data illustrated in the data display region 3002 and/or other data). For example, the data analysis region 3004 may display data representative of any of the computational methods disclosed herein. In some embodiments, the data display region 3002 and the data analysis region 3004 may be combined in the GUI 3000 (e.g., to include data output from a scientific instrument, and some analysis of the data, in a common graph or region).

The scientific instrument control region 3006 may include options that allow the user to control a scientific instrument (e.g., the scientific instrument 5010 discussed herein with reference to FIG. 15). For example, the scientific instrument control region 3006 may include options to control an experiment or other process that uses any of the novel compounds disclosed herein.

The settings region 3008 may include options that allow the user to control the features and functions of the GUI 3000 (and/or other GUIs) and/or perform common computing operations with respect to the data display region 3002 and data analysis region 3004 (e.g., saving data on a storage device, such as the storage device 4004 discussed herein with reference to FIG. 14, sending data to another user, labeling data, etc.).

FIG. 14 is a block diagram of a computing device 4000 that may perform some or all of the computational methods disclosed herein, in accordance with various embodiments. The computing device 4000 of FIG. 14 is illustrated as having a number of components, but any one or more of these components may be omitted or duplicated, as suitable for the application and setting. In some embodiments, some or all of the components included in the computing device 4000 may be attached to one or more motherboards and enclosed in a housing (e.g., including plastic, metal, and/or other materials). In some embodiments, some of these components may be fabricated onto a single system-on-a-chip (SoC) (e.g., an SoC may include one or more processing devices 4002 and one or more storage devices 4004). Additionally, in various embodiments, the computing device 4000 may not include one or more of the components illustrated in FIG. 14, but may include interface circuitry (not shown) for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 4000 may not include a display device 4010, but may include display device interface circuitry (e.g., a connector and driver circuitry) to which a display device 4010 may be coupled.

The computing device 4000 may include a processing device 4002 (e.g., one or more processing devices). As used herein, the term “processing device” may refer to any device or portion of a device that processes electronic data from registers and/or memory to transform that electronic data into other electronic data that may be stored in registers and/or memory. The processing device 4002 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

The computing device 4000 may include a storage device 4004 (e.g., one or more storage devices). The storage device 4004 may include one or more memory devices such as random access memory (RAM) (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 4004 may include memory that shares a die with a processing device 4002. In such an embodiment, the memory may be used as cache memory and may include embedded dynamic random access memory (eDRAM) or spin transfer torque magnetic random access memory (STT-MRAM), for example. In some embodiments, the storage device 4004 may include non-transitory computer readable media having instructions thereon that, when executed by one or more processing devices (e.g., the processing device 4002), cause the computing device 4000 to perform any appropriate ones of or portions of the methods disclosed herein.

The computing device 4000 may include an interface device 4006 (e.g., one or more interface devices 4006). The interface device 4006 may include one or more communication chips, connectors, and/or other hardware and software to govern communications between the computing device 4000 and other computing devices. For example, the interface device 4006 may include circuitry for managing wireless communications for the transfer of data to and from the computing device 4000. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although in some embodiments they might not. Circuitry included in the interface device 4006 for managing wireless communications may implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra mobile broadband (UMB) project (also referred to as “3GPP2”), etc.). In some embodiments, circuitry included in the interface device 4006 for managing wireless communications may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. In some embodiments, circuitry included in the interface device 4006 for managing wireless communications may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 4006 for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 4006 may include one or more antennas (e.g., one or more antenna arrays) to receipt and/or transmission of wireless communications.

In some embodiments, the interface device 4006 may include circuitry for managing wired communications, such as electrical, optical, or any other suitable communication protocols. For example, the interface device 4006 may include circuitry to support communications in accordance with Ethernet technologies. In some embodiments, the interface device 4006 may support both wireless and wired communication, and/or may support multiple wired communication protocols and/or multiple wireless communication protocols. For example, a first set of circuitry of the interface device 4006 may be dedicated to shorter-range wireless communications such as Wi-Fi or Bluetooth, and a second set of circuitry of the interface device 4006 may be dedicated to longer-range wireless communications such as global positioning system (GPS), EDGE, GPRS, CDMA, WiMAX, LTE, EV-DO, or others. In some embodiments, a first set of circuitry of the interface device 4006 may be dedicated to wireless communications, and a second set of circuitry of the interface device 4006 may be dedicated to wired communications.

The computing device 4000 may include battery/power circuitry 4008. The battery/power circuitry 4008 may include one or more energy storage devices (e.g., batteries or capacitors) and/or circuitry for coupling components of the computing device 4000 to an energy source separate from the computing device 4000 (e.g., AC line power).

The computing device 4000 may include a display device 4010 (e.g., multiple display devices). The display device 4010 may include any visual indicators, such as a heads-up display, a computer monitor, a projector, a touchscreen display, a liquid crystal display (LCD), a light-emitting diode display, or a flat panel display.

The computing device 4000 may include other input/output (I/O) devices 4012. The other I/O devices 4012 may include one or more audio output devices (e.g., speakers, headsets, earbuds, alarms, etc.), one or more audio input devices (e.g., microphones or microphone arrays), location devices (e.g., GPS devices in communication with a satellite-based system to receive a location of the computing device 4000, as known in the art), audio codecs, video codecs, printers, sensors (e.g., thermocouples or other temperature sensors, humidity sensors, pressure sensors, vibration sensors, accelerometers, gyroscopes, etc.), image capture devices such as cameras, keyboards, cursor control devices such as a mouse, a stylus, a trackball, or a touchpad, bar code readers, Quick Response (QR) code readers, or radio frequency identification (RFID) readers, for example.

The computing device 4000 may have any suitable form factor for its application and setting, such as a handheld or mobile computing device (e.g., a cell phone, a smart phone, a mobile internet device, a tablet computer, a laptop computer, a netbook computer, an ultrabook computer, a personal digital assistant (PDA), an ultra mobile personal computer, etc.), a desktop computing device, or a server computing device or other networked computing component.

One or more computing devices implementing any of the computational methods disclosed herein may be part of a scientific instrument support system. FIG. 15 is a block diagram of an example scientific instrument support system 5000 in which some or all of the computational methods disclosed herein may be performed, in accordance with various embodiments. The computational methods disclosed herein may be implemented by one or more of the scientific instrument 5010, the user local computing device 5020, the service local computing device 5030, or the remote computing device 5040 of the scientific instrument support system 5000.

Any of the scientific instrument 5010, the user local computing device 5020, the service local computing device 5030, or the remote computing device 5040 may include any of the embodiments of the computing device 4000 discussed herein with reference to FIG. 14, and any of the scientific instrument 5010, the user local computing device 5020, the service local computing device 5030, or the remote computing device 5040 may take the form of any appropriate ones of the embodiments of the computing device 4000 discussed herein with reference to FIG. 14.

The scientific instrument 5010, the user local computing device 5020, the service local computing device 5030, or the remote computing device 5040 may each include a processing device 5002, a storage device 5004, and an interface device 5006. The processing device 5002 may take any suitable form, including the form of any of the processing devices 4002 discussed herein with reference to FIG. 14, and the processing devices 5002 included in different ones of the scientific instrument 5010, the user local computing device 5020, the service local computing device 5030, or the remote computing device 5040 may take the same form or different forms. The storage device 5004 may take any suitable form, including the form of any of the storage devices 4004 discussed herein with reference to FIG. 14, and the storage devices 5004 included in different ones of the scientific instrument 5010, the user local computing device 5020, the service local computing device 5030, or the remote computing device 5040 may take the same form or different forms. The interface device 5006 may take any suitable form, including the form of any of the interface devices 4006 discussed herein with reference to FIG. 14, and the interface devices 5006 included in different ones of the scientific instrument 5010, the user local computing device 5020, the service local computing device 5030, or the remote computing device 5040 may take the same form or different forms.

The scientific instrument 5010, the user local computing device 5020, the service local computing device 5030, and the remote computing device 5040 may be in communication with other elements of the scientific instrument support system 5000 via communication pathways 5008. The communication pathways 5008 may communicatively couple the interface devices 5006 of different ones of the elements of the scientific instrument support system 5000, as shown, and may be wired or wireless communication pathways (e.g., in accordance with any of the communication techniques discussed herein with reference to the interface devices 4006 of the computing device 4000 of FIG. 14). The particular scientific instrument support system 5000 depicted in FIG. 15 includes communication pathways between each pair of the scientific instrument 5010, the user local computing device 5020, the service local computing device 5030, and the remote computing device 5040, but this “fully connected” implementation is simply illustrative, and in various embodiments, various ones of the communication pathways 5008 may be absent. For example, in some embodiments, a service local computing device 5030 may not have a direct communication pathway 5008 between its interface device 5006 and the interface device 5006 of the scientific instrument 5010, but may instead communicate with the scientific instrument 5010 via the communication pathway 5008 between the service local computing device 5030 and the user local computing device 5020 and the communication pathway 5008 between the user local computing device 5020 and the scientific instrument 5010.

The scientific instrument 5010 may include any appropriate scientific instrument, such as any of the instruments discussed herein with reference to any of the experiments or processes involving any of the novel compounds disclosed herein.

The user local computing device 5020 may be a computing device (e.g., in accordance with any of the embodiments of the computing device 4000 discussed herein) that is local to a user of the scientific instrument 5010. In some embodiments, the user local computing device 5020 may also be local to the scientific instrument 5010, but this need not be the case; for example, a user local computing device 5020 that is in a user's home or office may be remote from, but in communication with, the scientific instrument 5010 so that the user may use the user local computing device 5020 to control and/or access data from the scientific instrument 5010. In some embodiments, the user local computing device 5020 may be a laptop, smartphone, or tablet device. In some embodiments the user local computing device 5020 may be a portable computing device.

The service local computing device 5030 may be a computing device (e.g., in accordance with any of the embodiments of the computing device 4000 discussed herein) that is local to an entity that services the scientific instrument 5010. For example, the service local computing device 5030 may be local to a manufacturer of the scientific instrument 5010 or to a third-party service company. In some embodiments, the service local computing device 5030 may communicate with the scientific instrument 5010, the user local computing device 5020, and/or the remote computing device 5040 (e.g., via a direct communication pathway 5008 or via multiple “indirect” communication pathways 5008, as discussed above) to receive data regarding the operation of the scientific instrument 5010, the user local computing device 5020, and/or the remote computing device 5040 (e.g., the results of self-tests of the scientific instrument 5010, calibration coefficients used by the scientific instrument 5010, the measurements of sensors associated with the scientific instrument 5010, etc.). In some embodiments, the service local computing device 5030 may communicate with the scientific instrument 5010, the user local computing device 5020, and/or the remote computing device 5040 (e.g., via a direct communication pathway 5008 or via multiple “indirect” communication pathways 5008, as discussed above) to transmit data to the scientific instrument 5010, the user local computing device 5020, and/or the remote computing device 5040 (e.g., to update programmed instructions, such as firmware, in the scientific instrument 5010, to initiate the performance of test or calibration sequences in the scientific instrument 5010, to update programmed instructions, such as software, in the user local computing device 5020 or the remote computing device 5040, etc.). A user of the scientific instrument 5010 may utilize the scientific instrument 5010 or the user local computing device 5020 to communicate with the service local computing device 5030 to report a problem with the scientific instrument 5010 or the user local computing device 5020, to request a visit from a technician to improve the operation of the scientific instrument 5010, to order consumables or replacement parts associated with the scientific instrument 5010, or for other purposes.

The remote computing device 5040 may be a computing device (e.g., in accordance with any of the embodiments of the computing device 4000 discussed herein) that is remote from the scientific instrument 5010 and/or from the user local computing device 5020. In some embodiments, the remote computing device 5040 may be included in a datacenter or other large-scale server environment. In some embodiments, the remote computing device 5040 may include network-attached storage (e.g., as part of the storage device 5004). The remote computing device 5040 may store data generated by the scientific instrument 5010, perform analyses of the data generated by the scientific instrument 5010 (e.g., in accordance with programmed instructions), facilitate communication between the user local computing device 5020 and the scientific instrument 5010, and/or facilitate communication between the service local computing device 5030 and the scientific instrument 5010.

In some embodiments, one or more of the elements of the scientific instrument support system 5000 illustrated in FIG. 15 may not be present. Further, in some embodiments, multiple ones of various ones of the elements of the scientific instrument support system 5000 of FIG. 15 may be present. For example, a scientific instrument support system 5000 may include multiple user local computing devices 5020 (e.g., different user local computing devices 5020 associated with different users or in different locations). In another example, a scientific instrument support system 5000 may include multiple scientific instruments 5010, all in communication with service local computing device 5030 and/or a remote computing device 5040; in such an embodiment, the service local computing device 5030 may monitor these multiple scientific instruments 5010, and the service local computing device 5030 may cause updates or other information may be “broadcast” to multiple scientific instruments 5010 at the same time. Different ones of the scientific instruments 5010 in a scientific instrument support system 5000 may be located close to one another (e.g., in the same room) or farther from one another (e.g., on different floors of a building, in different buildings, in different cities, etc.). In some embodiments, a scientific instrument 5010 may be connected to an Internet-of-Things (IoT) stack that allows for command and control of the scientific instrument 5010 through a web-based application, a virtual or augmented reality application, a mobile application, and/or a desktop application. Any of these applications may be accessed by a user operating the user local computing device 5020 in communication with the scientific instrument 5010 by the intervening remote computing device 5040. In some embodiments, a scientific instrument 5010 may be sold by the manufacturer along with one or more associated user local computing devices 5020 as part of a local scientific instrument computing unit 5012.

In some embodiments, different ones of the scientific instruments 5010 included in a scientific instrument support system 5000 may be different types of scientific instruments 5010. In some such embodiments, the remote computing device 5040 and/or the user local computing device 5020 may combine data from different types of scientific instruments 5010 included in a scientific instrument support system 5000.

The following numbered paragraphs provide examples of embodiments disclosed herein.

Example 1 is a computational method, comprising detecting, using a computational device, a biomolecule-bound compound, wherein the biomolecular-bound compound is a combination of at least one biomolecule with a composition comprising at least one excipient and any of the novel compounds disclosed herein.

Example 2 includes the subject matter of Example 1, and further specifies that the detecting is based at least in part of data generated by an assay selected from fluorescence microscopy, flow cytometry, in vivo imaging, immunoassay, hybridization, chromatographic assay, electrophoretic assay, microwell plate based assay, fluorescence resonance energy transfer (FRET) system, bioluminescence resonance energy transfer (BRET) system, high throughput screening, or microarray.

Example 3 includes the subject matter of any of Examples 1 or 2, and further specifies that the detecting includes performing a machine-learning analysis technique.

Example 4 is one or more non-transitory computer-readable media having instructions thereon that, when executed by one or more processing devices of one or more computing devices, cause the one or more computing devices to perform the computational method of any of Examples 1-3.

Example 5 is a computing device configured to perform the computational method of any of Examples 1-3.

Example 6 is a system including a scientific instrument and a computing device, wherein the computing device is in communication with the scientific instrument, and the computing device is configured to perform the computational method of any of Examples 1-3 based on data generated by the scientific instrument.

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +1-5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A method, comprising:
determining an attribute, by a computing device, as a function of a machine-learning process, wherein data generated from one or more dyes and collected with an analyzer electronically connected to the computing device is inputted into the machine-learning process, and wherein the one or more dyes emit light in the far-red and/or near-IR region of the electromagnetic spectrum.

2. The method of claim 1, further comprising configuring the analyzer using a second machine-learning process.

3. The method of claim 1, wherein the attribute comprises a structure, a function, or an interaction.

4. The method of claim 1, wherein the collected data comprises a series of detectable responses from the one or more dyes.

5. The method of claim 4, wherein the detectable response is fluorescence emission or light scattering, or a combination thereof.

6. The method of claim 4, further comprising processing the data using the computing device to remove noise, correct for artifacts, and/or to normalize the data, such that the data is in a suitable format for subsequent analysis.

7. The method of claim 6, further comprising extracting one or more features from the processed data to capture detectable responses of interest.

8. The method of claim 7, further comprising training the machine learning process from the extracted features, such that the process learns patterns or relationships between the extracted features.

9. The method of claim 8, further comprising validating the trained machine learning process to assess the performance and ability of the process to accurately classify or predict patterns or relationships based on the detectable responses.

10. The method of claim 9, further comprising applying the trained process to a predict or classify detectable responses by making predictions based on the trained patterns or relationships.

11. The method of claim 1, wherein the analyzer is selected from the group consisting of a fluorescence microscope, flow cytometer, cell sorter, mass spectrometer, mass cytometer, imaging cytometer, fluorescence resonance energy transfer (FRET) system, bioluminescence resonance energy transfer (BRET) system, and an electron microscope.

12. The method of claim 1, wherein the analyzer is configured to collect data for an assay selected from the group consisting of an in vivo imaging assay, an immunoassay, a hybridization assay, a chromatographic assay, an electrophoretic assay, a microwell plate-based assay, a high throughput screening assay, and a microarray-based assay; or to collect data gene.

13. The method of claim 1, further comprising:
training, by the computing device, the machine-learning process, using training data correlating the data from the one or more fluorescent dyes to the attribute.

14. The method of claim 1, wherein the one or more dyes is bound to a biomolecule, wherein the biomolecule is selected from a protein, polypeptide, antibody, enzyme, nucleic acid, nucleoside triphosphate, oligonucleotide, biotin, hapten, cofactor, lectin, antibody binding protein, carotenoid, carbohydrate, hormone, neurotransmitter, growth factors, toxin, biological cell, lipid, receptor binding drug, fluorescent proteins, and a combination thereof.

15. The method of claim 1, wherein the one or more dyes emit light in the far-red and/or near-IR region of the electromagnetic spectrum upon excitation at an appropriate wavelength of light.

16. The method of claim 1, wherein the one or more dyes has an excitation profile in the far-red region of the electromagnetic spectrum.

17. The method of claim 1, wherein the one or more dyes has an excitation profile in the near-IR region of the electromagnetic spectrum.

18. The method of claim 1, wherein the one or more dyes is a cyanine compound.

19. The method of claim 1, wherein the one or more dyes is an azaindole cyanine compound.

20. The method of claim 1, wherein the one or more dyes is a compound of Table A.

21. One or more non-transitory computer-readable media having instructions thereon that, when executed by one or more processing devices of one or more computing devices, cause the one or more computing devices to perform the method of claim 1.

22. A computing device configured to perform the method of claim 1.

23. An apparatus comprising:
a computing device, wherein the computing device comprises:
at least one processor comprising memory, wherein the memory includes instructions to:
determine an attribute as a function of a machine-learning process, wherein data generated from one or more dyes and collected from an analyzer electronically connected to the computing device is inputted into the machine-learning process, wherein the one or more dyes emit light in the far-red and/or near-IR region of the electromagnetic spectrum.

24. The apparatus of claim 23, wherein the one or more dyes emit light in the far-red and/or near-IR region of the electromagnetic spectrum upon excitation at an appropriate wavelength of light.

25. The apparatus of claim 23, wherein the one or more dyes has an excitation profile in the far-red region of the electromagnetic spectrum.

26. The apparatus of claim 23, wherein the one or more dyes has an excitation profile in the near-IR region of the electromagnetic spectrum.

27. The apparatus of claim 23, wherein the one or more dyes is a cyanine compound.

28. The apparatus of claim 23, wherein the one or more dyes is an azaindole cyanine compound.

29. The apparatus of claim 23, wherein the one or more dyes is a compound of Table A.
